(19) United States
(12) Patent Application Publication
(10) Pub. No.: US 2024/0068040 A1
(43) Pub. Date: Feb. 29, 2024

(54) LAYERED ANALYSIS OF METHYLATED BIOMARKERS FOR USE IN CANCER DIAGNOSIS AND PROGNOSIS

(71) Applicant: The Board of Trustees of the Leland Stanford Junior University, Stanford, CA (US)

(72) Inventors: Shan X. WANG, Stanford, CA (US); Ritish PATNAIK, Stanford, CA (US)

(21) Appl. No.: 18/261,091

(22) PCT Filed: Jan. 11, 2022
(86) PCT No.: PCT/US22/11937

§ 371 Date: Jul. 11, 2023

Related U.S. Application Data
(60) Provisional application No. 63/136,507, filed on Jan. 12, 2021.

(51) Int. Cl. C12Q 1/6886

(52) U.S. Cl. CPC C12Q 1/6886; C12Q 2600/106; C12Q 2600/154

(57) ABSTRACT

A method of identifying methylated cell-free DNA (cfDNA) biomarkers is provided, referred to as layered analysis of methylated biomarkers (LAMB). In particular, the LAMB methodology can be used to analyze data from patients to discover methylated cfDNA biomarkers associated with cancer. LAMB was used to identify tumor suppressor candidates using meta-analysis of cancer tissue methylation studies, followed by screening for tumor-specific promoter CpGs in these tumor suppressors and analysis of microarray data for cancerous tissues, non-cancerous tissues adjacent to tumors, and healthy blood samples. Biomarker panels for diagnosis of liver, colorectal, prostate, and lung cancer as well as biomarkers for predicting tumor response to therapy are provided based on this LAMB methodology. The biomarkers identified by LAMB can be used alone or in combination with one or more additional biomarkers or relevant clinical parameters in prognosis, diagnosis, therapy selection, or monitoring treatment of cancer.

### BACKGROUND OF THE INVENTION

Minimally invasive blood tests for mutated cell-free DNA (cfDNA), fragmented DNA that freely circulates in the blood, are being tested for cancer detection, therapy selection, and therapy monitoring (Forshew et al. Sci. Transl. Med. 4, 136ra68 (2012); Dawson et al. N. Engl. J. Med. 368, 1199-1209 (2013); Bettegowda et al, Sci. Transl. Med. 6, 224ra24 (2014); Cohen et al., Science 359, 926-930 (2018); Corcoran & Chabner N. Engl. J. Med. 379, 1754-1765 (2018); McDonald et al. Sci. Transl. Med. 11, eaax7392 (2019)). Methylated cfDNA biomarkers have also shown promise for these applications, in part because of their intrinsic biological properties (Sun et al. Proc. Natl. Acad. Sci. U.S.A 112, E5503-E5512 (2015); Lamb & Dhillon Mol. Diagn. Ther. 21, 225-232 (2017); Xu et al. Nat. Mater. 16, 1155-1161 (2017); Liu et al. Ann. Oncol. 29, 1445-1453 (2018); Liu et al. Ann. Oncol. 31, 745-759 (2020); Luo et al. Sci. Transl. Med. 12, eaax7533 (2020)). Silencing of tumor suppressor genes through methylation of CpG dinucleotides in their promoter regions plays an important, early role in tumor development (Esteller Oncogene 21, 5427-5440 (2002); Jones & Baylin Nat. Rev. Genet. 3, 415-428 (2002)). Methylated DNA also wraps around histones, protecting it from degradation by nucleases in the blood (Snyder et al. Cell 164, 57-68 (2016); Mouliere et al. Sci. Transl. Med. 10, eaat4921 (2018)). From a technological angle, advances in targeted microarray and sequencing platforms have enabled the precise quantification of CpG methylation frequencies in cfDNA (Deng et al. Nat. Biotechnol. 27, 353-360 (2009); Sandoval et al. Epigenetics 6, 692-702 (2011)).

### SUMMARY OF THE INVENTION

A method of identifying methylated cell-free DNA (cfDNA) biomarkers is provided, referred to as layered analysis of methylated biomarkers (LAMB). In particular, the LAMB methodology can be used to analyze data from patients to discover methylated cfDNA biomarkers associated with cancer. LAMB was used to identify tumor suppressor candidates using meta-analysis of cancer tissue methylation studies, followed by screening for tumor-specific promoter CpGs in these tumor suppressors and analysis of microarray data for cancerous tissues, non-cancerous tissues adjacent to tumors, and healthy blood samples. Biomarker panels for diagnosis of liver, colorectal, prostate, and lung cancer as well as biomarkers for predicting tumor response to therapy are provided based on this LAMB methodology. The biomarkers identified by LAMB can be used alone or in combination with one or more additional biomarkers or relevant clinical parameters in prognosis, diagnosis, therapy selection, or monitoring treatment of cancer.

In one aspect, a method of diagnosing and treating hepatocellular carcinoma (HCC) in a patient is provided, the method comprising: a) obtaining a blood sample from the patient; b) measuring levels of methylation at one or more CpG sites in promoter regions of one or more biomarker genes in cell-free DNA (cfDNA) from the blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 compared to reference value ranges for methylation at the one or more CpG sites in cfDNA indicate that the patient has the HCC; and c) treating the patient for the HCC, if the patient has a positive diagnosis for the HCC based on the levels of methylation of the one or more CpG sites.

In certain embodiments, the one or more CpGs are selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864. In some embodiments, the method comprises measuring levels of methylation at cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864, and CpG sites located within 200 nucleotides thereof (locations of these CpG sites are given in the Illumina HumanMethylation450K Manifest).

Exemplary methods of treating a patient, having a positive diagnosis for HCC based on the levels of methylation of the one or more CpG sites, include, without limitation, surgical resection of an HCC tumor, liver transplantation, radiofrequency ablation, cryoablation, radiation therapy, chemotherapy, immunotherapy, or biologic therapy.

In certain embodiments, reference value ranges for methylation at the one or more CpG sites are obtained from cfDNA from one or more blood samples from one or more control subjects not having HCC.

In another aspect, a cell-free DNA methylated at one or more CpG sites selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864, and CpG sites located within 200 nucleotides thereof (locations of these CpG sites are given in the Illumina HumanMethylation450K Manifest) for use as a biomarker for diagnosis of hepatocellular carcinoma is provided.

In another aspect, an in vitro method of diagnosing hepatocellular carcinoma (HCC) in a patient is provided, the method comprising: a) obtaining a blood sample from the patient; and b) measuring levels of methylation at one or more CpG sites in promoter regions of one or more biomarker genes in cell-free DNA (cfDNA) from the blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 compared to reference value ranges for methylation at the one or more CpG sites in cfDNA indicate that the patient has the HCC.

In another aspect, a method of monitoring hepatocellular carcinoma (HCC) in a patient is provided, the method comprising: a) obtaining a first blood sample from the patient at a first time point and a second blood sample from the patient later at a second time point; and b) measuring levels of methylation at one or more CpG sites in promoter regions of one or more biomarker genes in cell-free DNA (cfDNA) from the first blood sample and the second blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA from the second blood sample compared to the cfDNA from the first blood sample indicate that the HCC is progressing, and detection of decreased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA from the second blood sample compared to the cfDNA from the first blood sample indicate that the HCC is not progressing.

In certain embodiments, the HCC is a primary tumor, a metastasis, or a recurrence.

In certain embodiments, the first time point is before a treatment of the patient for HCC is started and the second time point is during or after the treatment. In some embodiments, the treatment is surgical resection, liver transplantation, radiofrequency ablation, cryoablation, radiation therapy, chemotherapy, immunotherapy, or biologic therapy.

In certain embodiments, the method further comprises repeating steps a) and b).

In certain embodiments, the method further comprises increasing dosage or frequency of a treatment for HCC, changing to a different treatment, or starting palliative care for the patient if the HCC is progressing.

In another aspect, a method of monitoring for a recurrence of hepatocellular carcinoma (HCC) in a patient and treating the patient for the recurrence is provided, the method comprising: a) obtaining a first blood sample from the patient after treatment for a previous occurrence of HCC at a first time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from AK055957, APC, GSTP1, HOXA1, PFKP, PRDM2, RUNX3, SEPTIN9, SPINT2, and WIF1; c) obtaining a second blood sample from the patient at a second time point during a period of monitoring for the recurrence; d) measuring levels of methylation at the one or more CpG sites in promoter regions of the one or more biomarker genes in cfDNA from the second blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA of the second blood sample compared to the cfDNA of the first blood sample indicates that the HCC has recurred; e) treating the patient for the recurrence of the HCC, if the patient has a positive diagnosis for the recurrence of the HCC based on the levels of methylation of the one or more CpG sites; and f) repeating steps c)-e) subsequently during the period of monitoring for the recurrence.

In another aspect, a method of diagnosing and treating colorectal adenocarcinoma (CRC) in a patient is provided, the method comprising: a) obtaining a blood sample from the patient; b) measuring levels of methylation at one or more CpG sites in promoter regions of one or more biomarker genes in cell-free DNA (cfDNA) from the blood sample, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C compared to reference value ranges for methylation at the one or more CpG sites in cfDNA indicate that the patient has the CRC; and c) treating the patient for the CRC, if the patient has a positive diagnosis for the CRC based on the levels of methylation of the one or more CpG sites.

In certain embodiments, the one or more CpGs are selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409 and CpG sites located within 200 nucleotides thereof (locations of these CpG sites are given in the Illumina HumanMethylation450K Manifest). In some embodiments, the method comprises measuring levels of methylation at cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409.

Exemplary methods of treating a patient, having a positive diagnosis for CRC based on the levels of methylation of the one or more CpG sites, include, without limitation, surgical resection of a CRC tumor, radiation therapy, chemotherapy, immunotherapy, and biologic therapy.

In certain embodiments, reference value ranges for methylation at the one or more CpG sites are obtained from cfDNA from one or more blood samples from one or more control subjects not having CRC.

In another aspect, a cell-free DNA methylated at one or more CpG sites selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409 and CpG sites located within 200 nucleotides thereof (locations of these CpG sites are given in the Illumina HumanMethylation450K Manifest) for use as a biomarker for diagnosis of colorectal adenocarcinoma is provided.

In another aspect, an in vitro method of diagnosing colorectal adenocarcinoma (CRC) in a patient is provided, the method comprising: a) obtaining a blood sample from the patient; and b) measuring levels of methylation at one or more CpG sites in promoter regions of one or more biomarker genes in cell-free DNA (cfDNA) from the blood sample, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C compared to reference value ranges for methylation at the one or more CpG sites in cfDNA indicate that the patient has the CRC.

In another aspect, a method of monitoring colorectal adenocarcinoma (CRC) in a patient is provided, the method comprising: a) obtaining a first blood sample from the patient at a first time point and a second blood sample from the patient later at a second time point; and b) measuring levels of methylation at one or more CpG sites in promoter regions of one or more biomarker genes in cell-free DNA (cfDNA) from the first blood sample and the second blood sample, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C in the cfDNA from the second blood sample compared to the cfDNA from the first blood sample indicate that the CRC is progressing, and detection of decreased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C in the cfDNA from the second blood sample compared to the cfDNA from the first blood sample indicate that the CRC is not progressing.

In certain embodiments, the CRC is a primary tumor, a metastasis, or a recurrence.

In certain embodiments, the first time point is before a treatment of the patient for CRC is started and the second time point is during or after the treatment. In some embodiments, the treatment is surgical resection of a CRC tumor, radiation therapy, chemotherapy, immunotherapy, or biologic therapy.

In certain embodiments, the method further comprises increasing dosage or frequency of a treatment for CRC, changing to a different treatment for CRC, or starting palliative care for the patient if the CRC is progressing.

In certain embodiments, the method further comprises repeating steps a) and b).

In another aspect, a method of monitoring for a recurrence of colorectal adenocarcinoma (CRC) in a patient and treating the patient for the recurrence is provided, the method comprising: a) obtaining a first blood sample from the patient after treatment for a previous occurrence of CRC at a first time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C; c) obtaining a second blood sample from the patient at a second time point during a period of monitoring for the recurrence; d) measuring levels of methylation at one or more CpG sites in promoter regions of one or more biomarker genes in cfDNA from the second blood sample, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFPI2, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFPI2, TMEFF2, and UNC5C in the cfDNA of the second blood sample compared to the cfDNA of the first blood sample indicates that the CRC has recurred; e) treating the patient for the recurrence of the CRC, if the patient has a positive diagnosis for the recurrence of the CRC based on the levels of methylation of the one or more CpG sites; and f) repeating steps c)-e) subsequently during the period of monitoring for the recurrence.

In another aspect, a method of diagnosing and treating prostate adenocarcinoma (PRAD) in a patient is provided, the method comprising: a) obtaining a blood sample from the patient; b) measuring levels of methylation at one or more CpG sites in promoter regions of one or more biomarker genes in cell-free DNA (cfDNA) from the blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 compared to reference value ranges for methylation at the one or more CpG sites in cfDNA indicate that the patient has the PRAD; and c) treating the patient for the PRAD, if the patient has a positive diagnosis for the PRAD based on the levels of methylation of the one or more CpG sites.

In certain embodiments, the one or more CpGs are selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124. In some embodiments, the method comprises measuring levels of methylation at cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124 and CpG sites located within 200 nucleotides thereof (locations of these CpG sites are given in the Illumina HumanMethylation450K Manifest).

Exemplary methods of treating a patient, having a positive diagnosis for PRAD based on the levels of methylation of the one or more CpG sites, include, without limitation, radical prostatectomy, hormonal therapy, radiation therapy, brachytherapy, cryosurgery, high-intensity focused ultrasound, chemotherapy, immunotherapy, or biologic therapy.

In certain embodiments, reference value ranges for methylation at the one or more CpG sites are obtained from cfDNA from one or more blood samples from one or more control subjects not having PRAD.

In another aspect, a cell-free DNA methylated at one or more CpG sites selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124 and CpG sites located within 200 nucleotides thereof (locations of these CpG sites are given in the Illumina HumanMethylation450K Manifest) for use as a biomarker for diagnosis of prostate adenocarcinoma is provided.

In another aspect, an in vitro method of diagnosing prostate adenocarcinoma (PRAD) in a patient is provided, the method comprising: a) obtaining a blood sample from the patient; and b) measuring levels of methylation at one or more CpG sites in promoter regions of one or more biomarker genes in cell-free DNA (cfDNA) from the blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 compared to reference value ranges for methylation at the one or more CpG sites in cfDNA indicate that the patient has the PRAD.

In another aspect, a method of monitoring prostate adenocarcinoma (PRAD) in a patient is provided, the method comprising: a) obtaining a first blood sample from the patient at a first time point and a second blood sample from the patient later at a second time point; and b) measuring levels of methylation at one or more CpG sites in promoter regions of one or more biomarker genes in circulating free DNA (cfDNA) from the first blood sample and the second blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 in the cfDNA of the second blood sample compared to the cfDNA of the first blood sample indicate that the PRAD is progressing, and detection of decreased levels of methylation at the one or more CpG sites in the promoter regions of the one or more biomarker genes selected from APC, CD44, PYCARD, RARB, and RBP1 in the cfDNA of the second blood sample compared to the cfDNA of the first blood sample indicate that the PRAD is not progressing.

In certain embodiments, the PRAD is a primary tumor, a metastasis, or a recurrence.

In certain embodiments, the first time point is before a treatment of the patient for PRAD is started and the second time point is during or after the treatment. In some embodiments, the treatment is radical prostatectomy, hormonal therapy, radiation therapy, brachytherapy, cryosurgery, high-intensity focused ultrasound, chemotherapy, immunotherapy, or biologic therapy.

In certain embodiments, the method further comprises repeating steps a) and b).

In certain embodiments, the method further comprises increasing dosage or frequency of a treatment for PRAD, changing to a different treatment for PRAD, or starting palliative care for the patient if the PRAD is progressing.

In another aspect, a method of monitoring for a recurrence of prostate adenocarcinoma (PRAD) in a patient and treating the patient for the recurrence is provided, the method comprising: a) obtaining a first blood sample from the patient after treatment for a previous occurrence of PRAD at a first time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from APC, CD44, PYCARD, RARB, and RBP1; c) obtaining a second blood sample from the patient at a second time point during a period of monitoring for the recurrence; d) measuring levels of methylation at one or more CpG sites in promoter regions of the one or more biomarker genes in cfDNA from the second blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 in the cfDNA of the second blood sample compared to the cfDNA of the first blood sample indicates that the PRAD has recurred; e) treating the patient for the recurrence of the PRAD, if the patient has a positive diagnosis for the recurrence of the PRAD based on the levels of methylation of the one or more CpG sites; and f) repeating steps c)-e) subsequently during the period of monitoring for the recurrence.

In another aspect, a method of diagnosing and treating lung adenocarcinoma (LUAD) in a patient is provided, the method comprising: a) obtaining a blood sample from the patient; b) measuring levels of methylation at one or more CpG sites in promoter regions of one or more biomarker genes in cell-free DNA (cfDNA) from the blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 compared to reference value ranges for methylation at the one or more CpG sites in cfDNA indicate that the patient has the LUAD; and c) treating the patient for the LUAD, if the patient has a positive diagnosis for the LUAD based on the levels of methylation of the one or more CpG sites.

In certain embodiments, the one or more CpGs are selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105 and CpG sites located within 200 nucleotides thereof (locations of these CpG sites are given in the Illumina HumanMethylation450K Manifest). In some embodiments, the method comprises measuring levels of methylation at cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105.

Exemplary methods of treating a patient, having a positive diagnosis for LUAD based on the levels of methylation of the one or more CpG sites, include, without limitation, pneumonectomy, lobectomy, radiation therapy, chemotherapy, immunotherapy, or biologic therapy.

In certain embodiments, reference value ranges for methylation at the one or more CpG sites are obtained from cfDNA from one or more blood samples from one or more control subjects not having LUAD.

In another aspect, a cell-free DNA methylated at one or more CpG sites selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105 and CpG sites located within 200 nucleotides thereof (locations of these CpG sites are given in the Illumina HumanMethylation450K Manifest) for use as a biomarker for diagnosis of lung adenocarcinoma is provided.

In another aspect, an in vitro method of diagnosing lung adenocarcinoma (LUAD) in a patient is provided, the method comprising: a) obtaining a blood sample from the patient; and b) measuring levels of methylation at one or more CpG sites in promoter regions of one or more biomarker genes in cell-free DNA (cfDNA) from the blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 compared to reference value ranges for methylation at the one or more CpG sites in cfDNA indicate that the patient has the LUAD.

In another aspect, a method of monitoring lung adenocarcinoma (LUAD) in a patient is provided, the method comprising: a) obtaining a first blood sample from the patient at a first time point and a second blood sample from the patient later at a second time point; and b) measuring levels of methylation at one or more CpG sites in promoter regions of one or more biomarker genes in cell-free DNA (cfDNA) from the first blood sample and the second blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 in the cfDNA from the second blood sample compared to the cfDNA from the first blood sample indicate that the LUAD is progressing, and detection of decreased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 in the cfDNA from the second blood sample compared to the cfDNA from the first blood sample indicate that the LUAD is not progressing.

In certain embodiments, the LUAD is a primary tumor, a metastasis, or a recurrence.

In certain embodiments, the first time point is before a treatment of the patient for LUAD is started and the second time point is during or after the treatment. In some embodiments, the treatment is pneumonectomy, lobectomy, radiation therapy, chemotherapy, immunotherapy, or biologic therapy.

In certain embodiments, the method further comprises repeating steps a) and b).

In certain embodiments, the method further comprises increasing dosage or frequency of a treatment for LUAD, changing to a different treatment for LUAD, or starting palliative care for the patient if the LUAD is progressing.

In another aspect, a method of monitoring for a recurrence of lung adenocarcinoma (LUAD) in a patient and treating the patient for the recurrence is provided, the method comprising: a) obtaining a first blood sample from the patient after treatment for a previous occurrence of LUAD at a time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from AJAP1, CDO1, HOXA7, HOXA9, PTGDR, SOX17, TAC1, UNCX, ZIC4, and ZNF781; c) obtaining a second blood sample from the patient during a period of monitoring for the recurrence; d) measuring levels of methylation at one or more CpG sites in promoter regions of one or more biomarker genes in cfDNA from the second blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 in the cfDNA of the second blood sample compared to the cfDNA of the first blood sample indicates that the LUAD has recurred; e) treating the patient for the recurrence of the LUAD, if the patient has a positive diagnosis for the recurrence of the LUAD based on the levels of methylation of the one or more CpG sites; and f) repeating steps c)-e) subsequently during the period of monitoring for the recurrence.

In another aspect, a method of diagnosing and treating lung squamous cell carcinoma (LUSC) in a patient is provided, the method comprising: a) obtaining a blood sample from the patient; b) measuring levels of methylation at one or more CpG sites in promoter regions of one or more biomarker genes in cell-free DNA (cfDNA) from the blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 compared to reference value ranges for methylation at the one or more CpG sites in cfDNA indicate that the patient has the LUSC; and c) treating the patient for the LUSC, if the patient has a positive diagnosis for the LUSC based on the levels of methylation of the one or more CpG sites.

In certain embodiments, the CpGs are selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105 and CpG sites located within 200 nucleotides thereof (locations of these CpG sites are given in the Illumina HumanMethylation450K Manifest). In some embodiments, the method comprises measuring levels of methylation at cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105.

Exemplary methods of treating a patient, having a positive diagnosis for LUSC based on the levels of methylation of the one or more CpG sites, include, without limitation, pneumonectomy, lobectomy, radiation therapy, chemotherapy, immunotherapy, or biologic therapy.

In certain embodiments, reference value ranges for methylation at the one or more CpG sites are obtained from cfDNA from one or more blood samples from one or more control subjects not having LUSC.

In another aspect, a cell-free DNA methylated at one or more CpG sites selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105 and CpG sites located within 200 nucleotides thereof (locations of these CpG sites are given in the Illumina HumanMethylation450K Manifest) for use as a biomarker for diagnosis of lung squamous cell carcinoma is provided.

In another aspect, an in vitro method of diagnosing lung squamous cell carcinoma (LUSC) in a patient is provided, the method comprising: a) obtaining a blood sample from the patient; and b) measuring levels of methylation at one or more CpG sites in promoter regions of one or more biomarker genes in cell-free DNA (cfDNA) from the blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 compared to reference value ranges for methylation at the one or more CpG sites in cfDNA indicate that the patient has the LUSC.

In another aspect, a method of monitoring lung squamous cell carcinoma (LUSC) in a patient is provided, the method comprising: a) obtaining a first blood sample from the patient at a first time point and a second blood sample from the patient later at a second time point; and b) measuring levels of methylation at one or more CpG sites in promoter regions of one or more biomarker genes in circulating free DNA (cfDNA) from the first blood sample and the second blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 in the cfDNA from the second blood sample compared to the cfDNA from the first blood sample indicate that the LUSC is progressing, and detection of decreased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 in the cfDNA from the second blood sample compared to the cfDNA from the first blood sample indicate that the LUSC is not progressing.

In certain embodiments, the LUSC is a primary tumor, a metastasis, or a recurrence.

In certain embodiments, the first time point is before a treatment of the patient for LUSC is started and the second time point is during or after the treatment. In some embodiments, the treatment is pneumonectomy, lobectomy, radiation therapy, chemotherapy, immunotherapy, or biologic therapy.

In certain embodiments, the method further comprises repeating steps a) and b).

In certain embodiments, the method further comprises increasing dosage or frequency of a treatment for LUSC, changing to a different treatment for LUSC, or starting palliative care for the patient if the LUSC is progressing.

In another aspect, a method of monitoring for a recurrence of LUSC in a patient and treating the patient for the recurrence is provided, the method comprising: a) obtaining a first blood sample from the patient after treatment for a previous occurrence of LUSC at a time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from AJAP1, CDO1, HOXA7, HOXA9, PTGDR, SOX17, ZFP42, ZIC4, and ZNF781; c) obtaining a second blood sample from the patient during a period of monitoring for the recurrence; d) measuring levels of methylation at one or more CpG sites in promoter regions of one or more biomarker genes in cfDNA from the second blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 in the cfDNA of the second blood sample compared to the cfDNA of the first blood sample indicates that the LUSC has recurred; e) treating the patient for the recurrence of the LUSC, if the patient has a positive diagnosis for the recurrence of the LUSC based on the levels of methylation of the one or more CpG sites; and f) repeating steps c)-e) subsequently during the period of monitoring for the recurrence.

In the practice of the subject methods, any suitable method for measuring the levels of methylation at CpG sites may be used, including, without limitation, methylation-specific polymerase chain reaction (PCR), quantitative methylation-specific PCR, methylation-sensitive DNA restriction enzyme analysis, methylation-specific pyrosequencing, restriction enzyme-based sequencing, restriction enzyme-based microarray analysis, TET-assisted pyridine borane sequencing (TAPS), methylated DNA immunoprecipitation-based sequencing, methylated DNA immunoprecipitation-based microarray analysis, bisulfite sequencing, bisulfite microarray analysis, or methylation-specific giant magnetoresistive sensor-based microarray analysis.

### BRIEF DESCRIPTION OF THE DRAWINGS

FIGS. 1A-1B. Layered Analysis for Methylated Biomarkers (LAMB) workflow and validation. (FIG. 1A) Workflow and screening criteria for using meta-analysis and microarray data to discover methylated cfDNA biomarkers using LAMB. (FIGS. 1A-1B) LAMB biomarker screening process and independent cfDNA validation dataset for hepatocellular carcinoma (HCC) (FIG. 1A, right), colorectal adenocarcinoma (CRC) (FIG. 1B, left), and prostate adenocarcinoma (PRAD) tumors (FIG. 1B, right).

FIGS. 2A-2C. LAMB-HCC diagnostic performance in a cfDNA validation dataset. (FIG. 2A) Box-and-whisker plot of area under curve (AUC) values for methylation frequencies of LAMB-HCC CpGs and CpGs removed by LAMB Layers 1, 2, and 3. (FIG. 2B) Gene methylation frequency (β_{gene}) heatmap with unsupervised hierarchical clustering by gene. Rows indicate patients, and columns represent genes. β_{gene} is normalized across patients. (FIG. 2C) Receiver operating characteristic curves and corresponding panel sizes and AUCs for LAMB-HCC, meta-analysis, and microarray panels. **** P<0.0001; ** P<0.01.

FIG. 3. LAMB-CRC diagnostic performance in a cfDNA validation dataset. Receiver operating characteristic curves and corresponding number of CRC patients and AUCs for LAMB-CRC panel with all tumors, non-metastatic tumors, and early-stage tumors. Curves have 46 healthy patients as controls.

FIGS. 4A-4E. LAMB-PRAD therapy monitoring performance in a cfDNA validation dataset. (FIGS. 4A and 4B) Box-and-whisker plots of percent difference changes in first on-treatment visit score from baseline pre-treatment visit score (FIG. 4A) and corresponding ROC curves with panel sizes and AUCs (FIG. 4B) for LAMB-PRAD, meta-analysis, and microarray panels. (FIGS. 4C, 4D, and 4E) Plots of percent difference changes in LAMB-PRAD scores from baseline for patients with tumor progression (FIG. 4C), acquired therapy resistance (FIG. 4D), and delayed response (FIG. 4E). Light gray shading represents time periods with tumor progression per changes in PSA; Dark gray shading represents time periods with tumor response per changes in PSA. *** P<0.001; ** P<0.01.

FIGS. 5A-5C. LAMB-HCC, LAMB-CRC, and LAMB-PRAD methylation in tissues of other cancer types. (FIGS. 5A, 5B and 5C) Bar plots for methylation frequency of LAMB-HCC (FIG. 5A), LAMB-CRC (FIG. 5B) and LAMB-PRAD (FIG. 5C) panels in cancer types. Error bars indicate standard deviation, and values above error bars signify number of tumors. Red dashed lines show the threshold above which tumors are hypermethylated (βₚₐₙₑₗ>0.2). Cancer type names by abbreviation can be found in supplementary methods.

FIG. 6. Flow diagram of tissue study screening for LAMB-HCC meta-analysis. HCC abstracts and articles found through a search for “((((hepatocellular carcinoma) OR HCC) OR hepatoma) OR hepatocarcinoma) AND methylation” in PubMed and Embase were screened using the following criteria. PubMed identifiers for the included articles are in FIG. 9. MSP: Methylation-Specific Polymerase chain reaction; QMSP: Quantitative MSP.

FIG. 7. Flow diagram of tissue study screening for LAMB-CRC meta-analysis. CRC abstracts and articles found through a search for “((((((((colorectal adenocarcinoma) OR CRC) OR colon adenocarcinoma) OR rectal adenocarcinoma) OR colorectal carcinoma) OR colon carcinoma) OR rectal carcinoma) OR colorectal cancer) AND methylation” in PubMed and Embase were screened using the following criteria. PubMed identifiers for the included articles are in FIG. 10. MSP: Methylation-Specific Polymerase chain reaction; QMSP: Quantitative MSP

FIG. 8: Flow diagram of tissue study screening for LAMB-PRAD meta-analysis. PRAD abstracts and articles found through a search for “((((prostate adenocarcinoma) OR prostate cancer) OR prostate carcinoma) OR PRAD) AND methylation” in PubMed and Embase were screened using the following criteria. PubMed identifiers for included articles are in FIG. 11. PRAD: Prostate Adenocarcinoma; MSP: Methylation-Specific Polymerase chain reaction; QMSP: Quantitative MSP.

FIG. 9. Forest plots for genes identified by LAMB-HCC meta-analysis. Forest plots demonstrate diagnostic odds ratio (DOR), authors, and PubMed ID for each paper. The Hartung-Knapp-Sidik-Jonkman estimator was used to find adjusted DORs.

FIG. 10. Forest plots for genes identified by LAMB-CRC meta-analysis. Forest plots demonstrate diagnostic odds ratio (DOR), authors, and PubMed ID for each paper. The Hartung-Knapp-Sidik-Jonkman estimator was used to find adjusted DORs.

FIG. 11. Forest plots for genes identified by LAMB-PRAD meta-analysis. Forest plots demonstrate diagnostic odds ratio (DOR), authors, and PubMed ID for each paper. The Hartung-Knapp-Sidik-Jonkman estimator was used to find adjusted DORs.

FIG. 12. Box-and-whisker plot of β_{LAMB-HCC} scores for HCC tumors by stage. 450K data from 347 hepatocellular carcinoma (HCC) tumors and 50 adjacent non-cancerous tissues (ANTs) were downloaded from TCGA, and tumors were separated by stage. β_{LAMB-HCC} scores were calculated for all tissues. Two-sided Wilcoxon tests were used to compare LAMB panel scores among the tissue groups (**** P<0.0001).

FIG. 13. Box-and-whisker plot of β_{LAMB-CRC} scores for CRC tumors by stage. 450K data from 389 colorectal adenocarcinoma (CRC) tumors and 45 adjacent non-cancerous tissues (ANTs) were downloaded from TCGA, and tumors were separated by Gleason Score. β_{LAMB-CRC} scores were calculated for all tissues. Two-sided Wilcoxon tests were used to compare LAMB panel scores among the tissue groups (**** P<0.0001).

FIG. 14. Box-and-whisker plot of β_{LAMB-PRAD} scores for PRAD tumors by stage. 450K data from 502 prostate adenocarcinoma (PRAD) tumors and 50 adjacent non-cancerous tissues (ANTs) were downloaded from TCGA, and tumors were separated by Gleason Score. β_{LAMB-PRAD} scores were calculated for all tissues. Two-sided Wilcoxon tests were used to compare LAMB panel scores among the tissue groups (**** P<0.0001).

FIG. 15. Box-and-whisker plot of β_{LAMB-HCC}, β_{HCC meta-analysis}, and β_{HCC microarray} scores in HCC cfDNA data. The meta-analysis panel is comprised of 364 promoter CpGs in the 22 genes identified by LAMB Layer 1, while the microarray biomarker panel is comprised of 486 promoter CpGs in 267 genes found by applying LAMB Layers 2 and 3 to all of the promoter CpGs in the 450K microarray. βₚₐₙₑₗ scores for the LAMB-HCC, HCC meta-analysis, and HCC microarray panels were found. One-sided Wilcoxon tests were used to evaluate differences in βₚₐₙₑₗ scores between HCC+cirrhosis and cirrhosis patients (**** P<0.0001; *** P<0.001; ** P<0.01).

FIG. 16. Box-and-whisker plot of the AUCs of CpGs in LAMB-HCC, HCC meta-analysis, and HCC microarray panels. The meta-analysis panel is comprised of 364 promoter CpGs in the 22 genes identified by LAMB Layer 1, while the microarray biomarker panel is comprised of 486 promoter CpGs in 267 genes found by applying LAMB Layers 2 and 3 to all of the promoter CpGs in the 450K microarray. AUC values for CpGs in the LAMB-HCC, HCC meta-analysis, and HCC microarray panels were found. One-sided Wilcoxon tests were used to compare these panels' CpG AUCs (**** P<0.0001; ** P<0.01).

FIG. 17. ROC curve and dot plot of β_{LAMB-HCC} scores in HCC cfDNA data for patients with AFP<20 ng/mL. β_{LAMB-HCC} scores were calculated for 9 HCC and 19 cirrhosis patients with AFP serum values less than 20 ng/mL. A one-sided Wilcoxon test and ROC curve with corresponding AUC was used to evaluate these β_{LAMB-HCC} scores (** P<0.01).

FIG. 18. Box-and-whisker plot of the AUCs of CpGs in LAMB-PRAD and filtered out by LAMB Layers 1, 2, and 3. One-sided Wilcoxon tests were used to evaluate differences in CpG methylation frequency AUCs (**** P<0.0001; *** P<0.001).

FIG. 19. Heatmap of percent difference of gene methylation frequencies (β_{gene}) in first on-treatment visit from baseline, pre-treatment visit. Rows indicate patients, and columns represent genes. β_{gene} normalized across patients with unsupervised hierarchical clustering by LAMB-PRAD gene.

FIG. 20. Box-and-whisker plot of the AUCs of CpGs in LAMB-PRAD, PRAD meta-analysis, and PRAD microarray panels. The meta-analysis panel is comprised of 131 promoter CpGs in the nine genes identified by LAMB Layer 1, while the microarray biomarker panel is comprised of 1178 promoter CpGs in 624 genes found by applying LAMB Layers 2 and 3 to all of the promoter CpGs in the 450K microarray. AUC values for CpGs in the LAMB-PRAD, PRAD meta-analysis, and PRAD microarray panels were found. One-sided Wilcoxon tests were used to compare these panels' CpG AUCs (**** P<0.0001; ** P<0.01).

FIG. 21. Time-course plots of percent difference in β_{LAMB-PRAD} score from baseline visit for 20 additional patients. Blue indicates tumor response to therapy, and red indicates tumor progression. PSA decrease of greater than 30% indicated response from pre-treatment baseline visit to first on-treatment visit. For on-treatment visits past the first on-treatment visit, visits with PSA values that were less than the past visit, less than the patient's nadir PSA, or less than the clinical PSA cut-off of 4 ng/mL were considered time points when the tumor was responding, and visits with PSA values that were higher than the past visit, higher than the patient's peak PSA, and higher than the clinical cut-off were considered time points when the tumor was resistant.

FIGS. 22A-22C. LAMB panels' gene methylation in tissues of other cancer types. (FIGS. 22A, 22B and 22C) Heatmap of mean gene methylation frequencies (β_{gene, mean}) in primary tumors from 30 cancer types for LAMB-HCC (FIG. 22A), LAMB-CRC (FIG. 22B) and LAMB-PRAD (FIG. 22C) genes with unsupervised hierarchical clustering by gene. The rows indicate cancer types, and columns indicate genes. β_{gene, mean} is normalized across cancer types, and cancer types are ordered by their decreasing panel score.

FIG. 23. Heatmap for tumor type classification of HCC, CRC, and PRAD by LAMB panel score. Tumor type prediction was classified by the tumor's maximum LAMB panel score.

### DETAILED DESCRIPTION OF THE INVENTION

Compositions, methods, and kits are provided for diagnosing and treating cancer. In particular, methods of identifying methylated cell-free DNA (cfDNA) biomarkers associated with cancer are provided. The identified biomarkers can be used alone or in combination with one or more additional biomarkers or relevant clinical parameters in prognosis, diagnosis, therapy selection, or monitoring treatment of cancer.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a biomarker” includes a plurality of such biomarkers and reference to “the polypeptide” includes reference to one or more polypeptides and equivalents thereof, e.g. peptides or proteins known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “sample” as used herein relates to a material or mixture of materials, typically, although not necessarily, in liquid form, containing one or more analytes of interest.

As used herein, the term “circulating cell-free DNA” refers to DNA that is circulating in the peripheral blood of a patient. The DNA molecules in cell-free DNA may have a median size that is below 1 kb (e.g., in the range of 50 bp to 500 bp, 80 bp to 400 bp, or 100-1,000 bp), although fragments having a median size outside of this range may be present. Cell-free DNA may contain circulating tumor DNA (ctDNA), i.e., tumor DNA circulating freely in the blood of a cancer patient or circulating fetal DNA (if the subject is a pregnant female). cfDNA can be highly fragmented and in some cases can have a mean fragment size about 165-250 bp (Newman et al Nat Med. 2014 20: 548-54). cfDNA can be obtained by centrifuging whole blood to remove all cells, and then isolating the DNA from the remaining plasma or serum. Such methods are well known (see, e.g., Lo et al, Am J Hum Genet 1998; 62:768-75). Circulating cell-free DNA is double-stranded, but can be made single stranded by denaturation.

The term “biomarker” as used herein refers to a compound, such as cfDNA, a protein, a mRNA, a metabolite, or a metabolic byproduct which is differentially expressed or present at different concentrations, levels or frequencies in one sample compared to another, such as a biological sample (e.g., blood or tissue sample) from patients who have cancer compared to a biological sample from healthy control subjects (i.e., subjects not having cancer). Biomarkers include, but are not limited to, hepatocellular carcinoma (HCC) biomarkers including cfDNA methylated at one or more CpG sites in promoter regions of one or more biomarker genes selected from AK055957, APC, GSTP1, HOXA1, PFKP, PRDM2, RUNX3, SEPTIN9, SPINT2, and WIF1 (methylated CpG sites for each biomarker gene listed in Table 10); colorectal adenocarcinoma (CRC) biomarkers including cfDNA methylated at one or more CpG sites in promoter regions of one or more biomarker genes selected from ALX4, CNRIP1, COL4A1, COL4A2, EFEMP1, EYA4, FBN1, HIC, IKZF1, MIR34C, NDRG4, SDC2, SEPTIN9, SOX21, TFP12, TMEFF2, and UNC5C (methylated CpG sites for each biomarker gene listed in Table 11); prostate adenocarcinoma (PRAD) biomarkers including cfDNA methylated at one or more CpG sites in promoter regions of one or more biomarker genes selected from APC, CD44, PYCARD, RARB, and RBP1 (methylated CpG sites for each biomarker gene listed in Table 12); lung adenocarcinoma (LUAD) biomarkers including cfDNA methylated at one or more CpG sites in promoter regions of one or more biomarker genes selected from AJAP1, CDO1, HOXA7, HOXA9, PTGDR, SOX17, TAC1, UNCX, ZIC4, and ZNF781 (methylated CpG sites for each biomarker gene listed in Table 13); and lung squamous cell carcinoma (LUSC) biomarkers including cfDNA methylated at one or more CpG sites in promoter regions of one or more biomarker genes selected from AJAP1, CDO1, HOXA7, HOXA9, PTGDR, SOX17, ZFP42, ZIC4, and ZNF781 (methylated CpG sites for each biomarker gene listed in Table 14).

In some embodiments, the concentration, frequency, or level of a biomarker is determined before and after the administration of a treatment to a patient. The treatment may comprise, for example, without limitation, surgical resection of a tumor, radiation therapy, hormonal therapy, or administering an anti-cancer agent (e.g., chemotherapeutic agent, immunotherapeutic agent, or biologic agent) if the patient is diagnosed with cancer. The degree of change in the concentration, frequency, or level of a biomarker, or lack thereof, is interpreted as an indication of whether the treatment has the desired effect (e.g., anti-tumor activity). In other words, the concentration or level of a biomarker is determined before and after the administration of the treatment to an individual, and the degree of change, or lack thereof, in the level is interpreted as an indication of whether the individual is “responsive” to the treatment.

A “reference level” or “reference value” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in samples (e.g., methylation-specific polymerase chain reaction (PCR), quantitative methylation-specific PCR, methylation-sensitive DNA restriction enzyme analysis, methylation-specific pyrosequencing, or bisulfite genomic sequencing), where the levels of biomarkers may differ based on the specific technique that is used.

A “similarity value” is a number that represents the degree of similarity between two things being compared. For example, a similarity value may be a number that indicates the overall similarity between a patient's biomarker profile using specific phenotype-related biomarkers and reference value ranges for the biomarkers in one or more control samples or a reference profile (e.g., the similarity to an “HCC” biomarker expression profile, an “CRC” biomarker expression profile, a “PRAD” biomarker expression profile, a “LUAD” biomarker expression profile, or a “LUSC” biomarker expression profile). The similarity value may be expressed as a similarity metric, such as a correlation coefficient, or may simply be expressed as the difference in cfDNA methylation frequency or levels, or geometric mean scores for gene methylation frequency for methylated cfDNA biomarkers in a patient sample compared to a control cfDNA sample or reference cfDNA methylation profile.

The terms “quantity”, “amount”, and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

The term “cfDNA sample” with respect to an individual encompasses samples such as blood or plasma samples comprising cfDNA obtained from the individual. The cfDNA samples can be obtained by any suitable method such as by venipuncture. The definition also includes samples that have been manipulated in any way after their procurement, such as by treatment with reagents, washed, centrifuged, or enriched for particular types of molecules (e.g., methylated cfDNA biomarkers).

Obtaining and assaying a sample. The term “assaying” is used herein to include the physical steps of manipulating a sample to generate data related to the sample. As will be readily understood by one of ordinary skill in the art, a sample must be “obtained” prior to assaying the sample. Thus, the term “assaying” implies that the sample has been obtained. The terms “obtained” or “obtaining” as used herein encompass the act of receiving an extracted or isolated sample. For example, a testing facility can “obtain” a sample in the mail (or via delivery, etc.) prior to assaying the sample. In some such cases, the sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a sample from a subject. Accordingly, a sample can be isolated from a subject (and thus “obtained”) by the same person or same entity that subsequently assays the sample. When a sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a sample.

In some embodiments, the step of obtaining comprises the step of isolating a sample (e.g., a pre-treatment sample, a post-treatment sample, etc.). Methods and protocols for isolating various samples (e.g., a blood sample, a serum sample, a plasma sample, a biopsy sample, an aspirate, etc.) will be known to one of ordinary skill in the art and any convenient method may be used to isolate a sample.

It will be understood by one of ordinary skill in the art that in some cases, it is convenient to wait until multiple samples (e.g., a pre-treatment sample and a post-treatment sample) have been obtained prior to assaying the samples. Accordingly, in some cases an isolated sample (e.g., a pre-treatment sample, a post-treatment sample, etc.) is stored until all appropriate samples have been obtained. One of ordinary skill in the art will understand how to appropriately store a variety of different types of samples and any convenient method of storage may be used (e.g., refrigeration) that is appropriate for the particular sample. In some embodiments, a pre-treatment sample is assayed prior to obtaining a post-treatment sample. In some cases, a pre-treatment sample and a post-treatment sample are assayed in parallel. In some cases, multiple different post-treatment samples and/or a pre-treatment sample are assayed in parallel. In some cases, samples are processed immediately or as soon as possible after they are obtained.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the methylation level is less than or “greater than or equal to” a particular threshold, (the threshold can be pre-determined or can be determined by assaying a control sample). On the other hand, “assaying to determine the methylation level” can mean determining a quantitative value (using any convenient metric) that represents the level of methylation at a CpG site. The level of methylation can be expressed in arbitrary units associated with a particular assay (e.g., fluorescence units, e.g., mean fluorescence intensity (MFI)), or can be expressed as an absolute value with defined units (e.g., number of methylated CpG sites in a cfDNA gene, frequency of methylation at a CpG site in cfDNA, etc.). Additionally, the level of methylation at a CpG site can be compared to the methylation level of one or more additional CpG sites to derive a normalized value that represents a normalized methylation level. The specific metric (or units) chosen is not crucial as long as the same units are used (or conversion to the same units is performed) when evaluating multiple samples from the same individual (e.g., samples taken at different points in time from the same individual). This is because the units cancel when calculating a fold-change (i.e., determining a ratio) in the methylation level from one sample to the next (e.g., samples taken at different points in time from the same individual).

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7^{th} nucleotide in a nucleic acid molecule is methylated when the nucleotide present at the 7^{th} nucleotide in the nucleic acid molecule is 5-methylcytosine. Similarly, the methylation state of a cytosine at the 7^{th} nucleotide in a nucleic acid molecule is unmethylated when the nucleotide present at the 7^{th} nucleotide in the nucleic acid molecule is cytosine (and not 5-methylcytosine).

The methylation status can optionally be represented or indicated by a “methylation value” (e.g., representing a methylation frequency, fraction, ratio, percent, etc.). A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who not have recurrence. Differential methylation and specific levels or patterns of DNA methylation are prognostic and predictive biomarkers, e.g., once the correct cut-off or predictive characteristics have been defined.

The methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g, Antequera et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see, e.g., Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such a change in the nucleic acid molecule's nucleotide sequence can result in a nucleic acid molecule in which each methylated nucleotide is modified to a different nucleotide. Such a change in the nucleic acid nucleotide sequence can result in a nucleic acid molecule in which each unmethylated nucleotide is modified to a different nucleotide. Such a change in the nucleic acid nucleotide sequence can result in a nucleic acid molecule in which each of a selected nucleotide which is unmethylated (e.g., each unmethylated cytosine) is modified to a different nucleotide. Use of such a reagent to change the nucleic acid nucleotide sequence can result in a nucleic acid molecule in which each nucleotide that is a methylated nucleotide (e.g., each methylated cytosine) is modified to a different nucleotide. As used herein, use of a reagent that modifies a selected nucleotide refers to a reagent that modifies one nucleotide of the four typically occurring nucleotides in a nucleic acid molecule (C, G, T, and A for DNA and C, G, U, and A for RNA), such that the reagent modifies the one nucleotide without modifying the other three nucleotides. In one exemplary embodiment, such a reagent modifies an unmethylated selected nucleotide to produce a different nucleotide. In another exemplary embodiment, such a reagent can deaminate unmethylated cytosine nucleotides. An exemplary reagent is bisulfite.

As used herein, the term “bisulfite reagent” refers to a reagent comprising in some embodiments bisulfite, disulfite, hydrogen sulfite, or combinations thereof to distinguish between methylated and unmethylated cytidines, e.g., in CpG dinucleotide sequences.

The term “methylation assay” refers to any assay or method for determining the methylation state of one or more CpG dinucleotide sequences within a sequence of a nucleic acid. Exemplary methylation assays include, without limitation, bisulfite sequencing, Southern blotting using methyl-sensitive restriction enzymes, the methylation-sensitive arbitrarily-primed polymerase chain reaction (MS AP-PCR) assay, MethyLight™ assay, Digital MethyLight, HeavyMethyl™ assay, HeavyMethyl™ MethyLight™ assay, methylation-sensitive single nucleotide primer extension (Ms-SNuPE) assay, methylation-specific PCR (MSP) assay, combined bisulfite restriction analysis (COBRA) assay, the methylated CpG island amplification (MCA) assay, methylated CpG island amplification and microarray (MCAM), Hpall tiny fragment enrichment by ligation-mediated PCR (HELP) assay, HELP-seq assay, Glal hydrolysis and ligation adapter dependent PCR assay (GLAD-PCR) assay, methylated DNA immunoprecipitation-sequencing (MeDIP-Seq) assay, and methylated DNA immunoprecipitation-microarray analysis (MeDIP-chip) assay.

Bisulfite sequencing uses bisulfite treatment of DNA before sequencing to detect methylation sites. Treatment of DNA with bisulfite converts cytosine residues to uracil, but does not affect methylated cytosine residues. After bisulfite treatment, the only cytosines remaining in the DNA are methylated cytosines. Thus, sequencing the DNA after bisulfite treatment reveals the methylation status of individual cytosine residues at single-nucleotide resolution.

The MS AP-PCR assay uses methylation-sensitive restriction enzymes to digest DNA and PCR with CG-rich primers to selectively amplify regions that contain CpG dinucleotides (see, e.g., Gonzalgo et al. (1997) Cancer Research 57: 594-599, herein incorporated by reference).

The MethyLight assay uses bisulfite-dependent, quantitative fluorescence-based real-time PCR with methylation-specific priming and methylation-specific fluorescent probing for detection of DNA methylation. Digital MethyLight combines the MethyLight assay with digital PCR to allow detection of individual methylated molecules (see, e.g., Eads et al. (1999) Cancer Res. 59: 2302-2306, Campan et al. (2018) Methods Mol. Biol. 1708:497-513).

The HeavyMethy assay uses methylation specific blocking probes (also referred to herein as blockers) covering CpG positions between, or covered by, amplification primers to enable methylation-specific selective amplification of a nucleic acid sample.

The HeavyMethyl MethyLight assay is a variation of the MethyLight™ assay, wherein the MethyLight™ assay is combined with methylation specific blocking probes covering CpG positions between the amplification primers.

The Ms-SNuPE assay uses bisulfite treatment of DNA combined with PCR using primers designed to hybridize immediately upstream of the CpG site(s) and electrophoresis of amplicons on polyacrylamide gels for visualization and quantitation. Treatment of DNA (genomic or cfDNA) with sodium bisulfite causes unmethylated cytosines to be converted to uracils, During the PCR step, uracil is replicated as thymine, and methylcytosine is replicated as cytosine during amplification. The ratio of methylated versus unmethylated cytosine (C versus T) at the original CpG sites can be determined by incubating the gel-isolated PCR product, primer(s), and Taq polymerase with either [³²P]dCTP or [³²P]TTP followed by denaturing polyacrylamide gel electrophoresis and phosphorimage analysis. Ms-SNuPE primers can also be designed to incorporate either [³²P]dATP or [³²P]dGTP into the opposite strand to assess methylation status depending on which CpG site is analyzed (see, e.g., Gonzalgo & Jones (1997) Nucleic Acids Res. 25: 2529-2531; Gonzalgo et al. (2007) Nat. Protoc. 2(8):1931-6).

The MSP assay uses bisulfite treatment of DNA for conversion of non-methylated cytosines to uracils and subsequent amplification with primers specific for methylated versus unmethylated DNA (see, e.g., Herman et al. (1996) Proc. Natl. Acad. Sci. USA 93: 9821-9826, and U.S. Pat. No. 5,786,146).

The COBRA assay uses bisulfite treatment of DNA for conversion of non-methylated cytosines to uracils, locus-specific PCR amplification of the bisulfite-converted DNA, restriction digestion, electrophoresis an analysis of restriction patterns on a gel (see, e.g., Xiong & Laird (1997) Nucleic Acids Res. 25: 2532-2534; Bilichak et al. (2017) Methods Mol. Biol. 1456:63-71).

The MCA assay uses methylation-sensitive restriction enzymes to digest DNA, followed by adaptor ligation and PCR to selectively amplify methylated CpG-rich sequences (see, e.g., Toyota et al. (1999) Cancer Res. 59: 2307-12, and WO 00/26401A1).

The MCAM assay uses MCA in combination with a CpG island microarray to detect DNA methylation in a high-throughput fashion (see, e.g., Estecio et al. (2007) Genome Res. 17(10):1529-1536).

The HELP assay uses the methylation-sensitive restriction enzyme, Hpall, to cut DNA, and a methylation-insensitive isoschizomer, Mspl, as a control. Microarray analysis is performed with microarrays containing probes designed to detect the Hpall/Mspl fragments. HELP-seq combines the HELP assay with massively parallel sequencing of DNA methylation sites (see, e.g., Greally (2018) Methods Mol. Biol. 1708:191-207; Suzuki et al. (2010) Methods 52(3):218-22).

The GLAD-PCR assay uses a site-specific methyl-directed DNA-endonucleases that cleave only methylated DNA, followed by ligation of DNA fragments to universal adapters for high-throughput PCR (see, e.g., Malyshev et al. (2020) Acta Naturae 12(3):124-133; Russian Patent RU 2525710).

The MeDIP assay uses an antibody against 5-methylcytosine to immunoprecipitate methylated DNA fragments. This technique can be combined with high-throughput DNA detection methods using microarray hybridization (MeDIP-chip) or next-generation sequencing (MeDIP-seq). See, e.g., Weber et al. (2005) Nat. Genet. 37 (8): 853-862, Palmke et al. (2011) Methods 53(2):175-184, Quackenbush et al. (2008) Cancer Res. 68(6):1786-1796, Zhu et al. (2019) Analyst 144(6):1904-1915, Yang et al. (2014) Life Sci. 113(1-2):45-54.

Southern Blotting can also be used to detect DNA methylation. The DNA is first digested with methylation-sensitive restriction enzymes, and the restriction fragments are analyzed by Southern Blot.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The terms “methylation-specific restriction enzyme” or “methylation-sensitive restriction enzyme” refers to an enzyme that selectively digests a nucleic acid dependent on the methylation state of its recognition site. In the case of a restriction enzyme that specifically cuts if the recognition site is not methylated or is hemimethylated, the cut will not take place or will take place with a significantly reduced efficiency if the recognition site is methylated. In the case of a restriction enzyme that specifically cuts if the recognition site is methylated, the cut will not take place or will take place with a significantly reduced efficiency if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, a “different nucleotide” refers to a nucleotide that is chemically different from a selected nucleotide, typically such that the different nucleotide has Watson-Crick base-pairing properties that differ from the selected nucleotide, whereby the typically occurring nucleotide that is complementary to the selected nucleotide is not the same as the typically occurring nucleotide that is complementary to the different nucleotide. For example, when C is the selected nucleotide, U or T can be the different nucleotide, which is exemplified by the complementarity of C to G and the complementarity of U or T to A. As used herein, a nucleotide that is complementary to the selected nucleotide or that is complementary to the different nucleotide refers to a nucleotide that base-pairs, under high stringency conditions, with the selected nucleotide or different nucleotide with higher affinity than the complementary nucleotide's base-paring with three of the four typically occurring nucleotides. An example of complementarity is Watson-Crick base pairing in DNA (e.g., A-T and C-G) and RNA (e.g., A-U and C-G). Thus, for example, G base-pairs, under high stringency conditions, with higher affinity to C than G base-pairs to G, A, or T and, therefore, when C is the selected nucleotide, G is a nucleotide complementary to the selected nucleotide.

As used herein, the “sensitivity” of a given marker refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

The term “AUC” as used herein is an abbreviation for the “area under a curve”. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better, the optimum is 1; a random test would have a ROC curve lying on the diagonal with an area of 0.5; for reference: J. P. Egan. (1975) Signal Detection Theory and ROC Analysis, Academic Press, New York).

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

Additional Terms.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer) as well as those in which prevention is desired (e.g., those with a genetic predisposition to developing cancer, those with an environmental exposure to a carcinogen, or who otherwise have an increased susceptibility or increased likelihood of developing cancer, those suspected of having cancer, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of sample(s) was used and/or how it was used, the technique used to assay the sample, the results of the assay (e.g., the level of CpG methylation as measured and/or the fold-change of a level of CpG methylation over time or in a post-treatment assay compared to a pre-treatment assay), the assessment as to whether the individual is determined to have cancer, a recommendation for treatment (e.g., a particular anti-cancer therapy), and/or to continue or alter therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

It will be apparent to one of ordinary skill in the art that various changes and modifications can be made without departing from the spirit or scope of the invention.

Methylated cfDNA Biomarkers and Diagnostic Methods

Hypermethylation of CpG-islands in regulatory regions of promoters and/or the first exons in a variety of genes is associated with a variety of cancers. Layered analysis of methylated biomarkers (LAMB) was used to identify methylated cell-free DNA (cfDNA) biomarkers associated with hepatocellular carcinoma (HCC), colorectal adenocarcinoma (CRC), prostate adenocarcinoma (PRAD), lung adenocarcinoma (LUAD), and lung squamous cell carcinoma (LUSC).

The identified HCC biomarkers include cfDNA methylated at one or more CpG sites in promoter regions of the genes, cfDNA methylated at one or more CpG sites in promoter regions of one or more biomarker genes selected from AK055957, APC, GSTP1, HOXA1, PFKP, PRDM2, RUNX3, SEPTIN9, SPINT2, and WIF1 (methylated CpG sites for each biomarker gene listed in Table 10). Increased frequency or levels of methylation at CpG sites in these biomarkers genes are commonly found in HCC tumors. In particular, increased frequency or levels of methylation at one or more CpG sites selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 (locations of these CpG sites are given in the Illumina HumanMethylation450K Manifest), and CpG sites located within 200 nucleotides thereof is associated with HCC. Accordingly, monitoring the frequency or levels of methylation of these CpG sites is useful for prognosis, diagnosis, therapy selection, and monitoring treatment of HCC.

The identified CRC biomarkers include cfDNA methylated at one or more CpG sites in promoter regions of the genes, cfDNA methylated at one or more CpG sites in promoter regions of one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C (methylated CpG sites for each biomarker gene listed in Table 11). Increased frequency or levels of methylation at CpG sites in these biomarkers genes are commonly found in CRC tumors. In particular, increased frequency or levels of methylation at one or more CpG sites selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409 (locations of these CpG sites are given in the Illumina HumanMethylation450K Manifest), and CpG sites located within 200 nucleotides thereof is associated with CRC. Accordingly, monitoring the frequency or levels of methylation of these CpG sites is useful for prognosis, diagnosis, therapy selection, and monitoring treatment of CRC.

The identified PRAD biomarkers include cfDNA methylated at one or more CpG sites in promoter regions of the genes, cfDNA methylated at one or more CpG sites in promoter regions of one or more biomarker genes selected from APC, CD44, PYCARD, RARB, and RBP1 (methylated CpG sites for each biomarker gene listed in Table 12). Increased frequency or levels of methylation at CpG sites in these biomarkers genes are commonly found in PRAD tumors. In particular, increased frequency or levels of methylation at one or more CpG sites selected cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124 (locations of these CpG sites are given in the Illumina HumanMethylation450K Manifest), and CpG sites located within 200 nucleotides thereof is associated with PRAD. Accordingly, monitoring the frequency or levels of methylation of these CpG sites is useful for prognosis, diagnosis, therapy selection, and monitoring treatment of PRAD.

The identified LUAD biomarkers include cfDNA methylated at one or more CpG sites in promoter regions of the genes, cfDNA methylated at one or more CpG sites in promoter regions of one or more biomarker genes selected from AJAP1, CDO1, HOXA7, HOXA9, PTGDR, SOX17, TAC1, UNCX, ZIC4, and ZNF781 (methylated CpG sites for each biomarker gene listed in Table 13). Increased frequency or levels of methylation at CpG sites in these biomarkers genes are commonly found in LUAD tumors. In particular, increased frequency or levels of methylation at one or more CpG sites selected cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105 (locations of these CpG sites are given in the Illumina HumanMethylation450K Manifest), and CpG sites located within 200 nucleotides thereof is associated with LUAD. Accordingly, monitoring the frequency or levels of methylation of these CpG sites is useful for prognosis, diagnosis, therapy selection, and monitoring treatment of LUAD.

The identified LUSC biomarkers include cfDNA methylated at one or more CpG sites in promoter regions of the genes, cfDNA methylated at one or more CpG sites in promoter regions of one or more biomarker genes selected from AJAP1, CDO1, HOXA7, HOXA9, PTGDR, SOX17, ZFP42, ZIC4, and ZNF781 (methylated CpG sites for each biomarker gene listed in Table 14). Increased frequency or levels of methylation at CpG sites in these biomarkers genes are commonly found in LUSC tumors. In particular, increased frequency or levels of methylation at one or more CpG sites selected cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105 (locations of these CpG sites are given in the Illumina HumanMethylation450K Manifest), and CpG sites located within 200 nucleotides thereof is associated with LUSC. Accordingly, monitoring the frequency or levels of methylation of these CpG sites is useful for prognosis, diagnosis, therapy selection, and monitoring treatment of LUSC.

In certain embodiments, a panel of methylated cfDNA biomarkers for use in diagnosis of HCC, CRC, PRAD, LUAD, or LUSC, or any combination thereof, is provided. Biomarker panels of any size can be used in the practice of the subject methods. Biomarker panels typically comprise at least 2 methylated cfDNA biomarkers and up to 20 methylated cfDNA biomarkers, including any number of biomarkers in between, such as 2, 3, 4, 5, 6, 7, 8, 9, 10, 11, 12, 13, 14, 15, 16, 17, 18, 19, or 20 methylated cfDNA biomarkers. In certain embodiments, the biomarker panel comprises at least 2, or at least 3, or at least 4, or at least 5, or at least 6, or at least 7, or at least 8, or at least 9, or at least 10, or at least 11, or at least 12, or at least 13, or at least 14, or at least 5, or at least 16, or at least 17, or at least 18, or at least 19, or at least 20 or more methylated cfDNA biomarkers. Although smaller biomarker panels are usually more economical, larger biomarker panels (i.e., greater than 20 biomarkers) have the advantage of providing more detailed information and can also be used in the practice of the subject methods.

A sample comprising methylated cfDNA (i.e., a “cfDNA sample”) is obtained from the subject. The sample is typically a blood or plasma sample comprising cfDNA taken from the subject. A “control” sample, as used herein, refers to a cfDNA sample from a subject that is not diseased. That is, a control sample is obtained from a normal or healthy subject (e.g., an individual known to not have HCC, CRC, PRAD, LUAD, or LUSC, or other type of cancer). A cfDNA sample can be obtained from a subject by conventional techniques. For example, blood samples can be obtained by venipuncture according to methods well known in the art.

When analyzing the frequency or levels of methylation at CpG sites in a cfDNA sample from a subject, the reference value ranges used for comparison can represent the frequency or levels of DNA methylation at CpG sites in a cfDNA sample from one or more subjects without HCC, CRC, PRAD, LUAD, or LUSC (i.e., normal or healthy control). Alternatively, the reference values can represent the frequency or levels of methylation at CpG sites in cfDNA samples from one or more subjects with HCC, CRC, PRAD, LUAD, or LUSC, wherein similarity to the reference value ranges indicates the subject has HCC, CRC, PRAD, LUAD, or LUSC.

In some cases, combinations of methylated cfDNA biomarkers are used in the subject methods. In some such cases, the levels of all measured biomarkers must change (as described above) in order for the diagnosis to be made. In some embodiments, only some biomarkers are used in the methods described herein. For example, a single biomarker, 2 biomarkers, 3 biomarkers, 4 biomarkers, 5 biomarkers, 6 biomarkers, 7 biomarkers, 8 biomarkers, 9 biomarkers, 10 biomarkers, 11 biomarkers, 12 biomarkers, 13 biomarkers, 14 biomarkers, 15 biomarkers, 16 biomarkers, 17 biomarkers, 18 biomarkers, 19 biomarkers, or 20 biomarkers can be used in any combination. In other embodiments, all the biomarkers are used. The quantitative values may be combined in linear or non-linear fashion to calculate one or more risk scores for HCC, CRC, PRAD, LUAD, or LUSC for the individual.

In some embodiments, a geometric mean score is calculated from the gene methylation frequency profile for 2, 3, 4, 5, 6, 7, 8, 9, or all 10 of the AK055957, APC, GSTP1, HOXA1, PFKP, PRDM2, RUNX3, SEPTIN9, SPINT2, and WIF1 genes, wherein the geometric mean score indicates whether or not the individual has HCC. The geometric mean score may further distinguish between a subject who has HCC versus a subject who does not have HCC.

In some embodiments, a geometric mean score is calculated from the gene methylation frequency profile for 2, 3, 4, 5, 6, 7, 8, 9, 10, 11, 12, 13, 14, 15, 16, or all 17 of the ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C genes, wherein the geometric mean score indicates whether or not the individual has CRC. The geometric mean score may further distinguish between a subject who has CRC versus a subject who does not have CRC.

In some embodiments, a geometric mean score is calculated from the gene methylation frequency profile for 2, 3, 4, or all 5 of the APC, CD44, PYCARD, RARB, and RBP1 genes, wherein the geometric mean score indicates whether or not the individual has PRAD. The geometric mean score may further distinguish between a subject who has PRAD versus a subject who does not have PRAD.

In some embodiments, a geometric mean score is calculated from the gene methylation frequency profile for 2, 3, 4, 5, 6, 7, 8, 9, or all 10 of the AJAP1, CDO1, HOXA7, HOXA9, PTGDR, SOX17, TAC1, UNCX, ZIC4, and ZNF781 genes, wherein the geometric mean score indicates whether or not the individual has LUAD. The geometric mean score may further distinguish between a subject who has LUAD versus a subject who does not have LUAD.

In some embodiments, a geometric mean score is calculated from the gene methylation frequency profile for 2, 3, 4, 5, 6, 7, 8, 9, or all 10 of the AJAP1, CDO1, HOXA7, HOXA9, PTGDR, SOX17, ZFP42, ZIC4, and ZNF781 genes, wherein the geometric mean score indicates whether or not the individual has LUSC. The geometric mean score may further distinguish between a subject who has LUSC versus a subject who does not have LUSC.

The methods described herein may be used to determine an appropriate treatment regimen for a patient and, in particular, whether a patient should be treated for HCC, CRC, PRAD, LUAD, or LUSC. For example, a patient is selected for treatment for HCC, CRC, PRAD, LUAD, or LUSC if the patient has a positive diagnosis for HCC, CRC, PRAD, LUAD, or LUSC based on a cfDNA methylation profile, as described herein. In some cases, the diagnostic methods described herein may be used by themselves or combined with medical imaging to confirm the diagnosis and further evaluate the extent of cancerous disease (how far and where the cancer has spread) to aid in determining prognosis and evaluating optimal strategies for treatment (e.g., surgery, radionuclide therapy, chemotherapy, targeted therapy, immunotherapy, biologic therapy, etc.). Exemplary medical imaging techniques include, without limitation, magnetic resonance imaging (MRI), positron emission tomography (PET), single photon emission computed tomography (SPECT), computed tomography (CT), ultrasound imaging (UI), optical imaging (01), photoacoustic imaging (PI), fluoroscopy, and fluorescence imaging.

In some embodiments, the HCC methylated cfDNA biomarkers are used in combination with other biomarkers for diagnosing HCC, such as alpha-fetoprotein (AFP) or des-gamma carboxyprothrombin (DCP). For example, blood levels of AFP or DCP or a combination thereof can be monitored in addition to methylation of the cfDNA biomarkers.

In some embodiments, the CRC methylated cfDNA biomarkers are used in combination with other biomarkers for diagnosing CRC. For example, the KRAS, NRAS, or BRAF proto-oncogenes, human epidermal growth factor receptor 2 (HER2), carcinoembryonic antigen (CEA), or NTRK fusions, or a combination thereof can be monitored in addition to methylation of the cfDNA biomarkers.

In some embodiments, the PRAD methylated cfDNA biomarkers are used in combination with other biomarkers for diagnosing PRAD. For example, prostate-specific antigen (PSA), PCA3, kallikrein, HOXC6, DLX1, or TMPRSS2-ERG fusions, or a combination thereof can be monitored in addition to methylation of the cfDNA biomarkers.

In some embodiments, the LUAD methylated cfDNA biomarkers are used in combination with other biomarkers for diagnosing LUAD. For example, EGFR, ALK, mutated BRAF (e.g., V600E), KRAS, MET, HER2, PIK3, ROS1, RET, or NTRK fusions, or a combination thereof can be monitored in addition to methylation of the cfDNA biomarkers.

In some embodiments, the LUSC methylated cfDNA biomarkers are used in combination with other biomarkers for diagnosing LUSC. For example, p40, PD-L1, and cytokeratin 5/6, EGFR, KRAS, MET, FGFR, PIK3CA, DDR2, PTEN deletion, or a combination thereof can be monitored in addition to methylation of the cfDNA biomarkers.

Exemplary treatments for HCC include, without limitation, tumor surgical resection, radiofrequency ablation (RFA), cryoablation, percutaneous ethanol or acetic acid injection, transcatheter arterial chemoembolization (TACE), selective internal radiation therapy (SIRT), high intensity focused ultrasound, or external beam therapy, liver transplantation, portal vein embolization, or administering anti-cancer therapeutic agents such as chemotherapeutic agents (e.g., cisplatin, gemcitabine, oxaliplatin, doxorubicin, 5-fluorouracil, capecitabine, or mitoxantrone), targeted therapeutic agents (e.g., sorafenib, regorafenib, lenvatinib, or cabozantinib), immunotherapeutic agents (e.g., ramucirumab, nivolumab, or pembrolizumab), or radioisotopes (e.g., Yttrium-90, Iodine-131, Rhenium-188, or Holmium-166), or a combination thereof.

Exemplary treatments for CRC include, without limitation, tumor surgical resection such as endoscopic mucosal resection or endoscopic submucosal dissection; radiation therapy, administering anti-cancer therapeutic agents such as chemotherapeutic agents (e.g., fluorouracil, capecitabine, oxaliplatin, irinotecan, or Tegafur/uracil (UFT)), targeted therapeutic agents (e.g., epidermal growth factor receptor inhibitors such as aflibercept, cetuximab, or panitumumab), antiangiogenic drugs (e.g., bevacizumab), or a combination thereof.

Exemplary treatments for PRAD include, without limitation, radical prostatectomy, transurethral resection of the prostate, external beam radiation therapy, brachytherapy, cryosurgery, high-intensity focused ultrasound, administering anti-cancer therapeutic agents such as hormonal therapeutic agents (e.g., antiandrogens such as abiraterone, apalutamide, enzalutamide, bicalutamide, nilutamide, topilutamide, ketoconazole, abiraterone acetate, or seviteronel), chemotherapeutic agents (e.g., cabazitaxel, bevacizumab, docetaxel, thalidomide, or prednisone), or immunotherapeutic agents (e.g., Sipuleucel-T cancer vaccine), or a combination thereof.

Exemplary treatments for LUAD include, without limitation, pneumonectomy, lobectomy, radiation therapy, administering anti-cancer therapeutic agents such as chemotherapeutic agents (e.g., cisplatin or carboplatin), targeted therapeutic agents (e.g., tyrosine kinase inhibitors such as gefitinib, erlotinib, afatinib, dacomitinib, or osimertinib; ALK inhibitors such as crizotinib, ceritinib, alectinib or brigatinib), immunotherapeutic agents (e.g., immune checkpoint inhibitors such as nivolumab or pembrolizumab), or a combination thereof.

Exemplary treatments for LUAD include, without limitation, pneumonectomy, lobectomy, radiation therapy, administering anti-cancer therapeutic agents such as chemotherapeutic agents (e.g., cisplatin or carboplatin), targeted therapeutic agents (e.g., tyrosine kinase inhibitors such as gefitinib, erlotinib, afatinib, dacomitinib, or osimertinib; ALK inhibitors such as crizotinib, ceritinib, alectinib or brigatinib), immunotherapeutic agents (e.g., immune checkpoint inhibitors such as nivolumab or pembrolizumab), or a combination thereof.

The cfDNA biomarkers can be used for monitoring HCC, CRC, PRAD, LUAD, or LUSC in a patient. For example, for monitoring HCC, a first cfDNA sample can be obtained from the patient at a first time point and a second cfDNA sample can be obtained from the patient at a second time point. In some embodiments, the patient is monitored for HCC by detecting methylation at one or more CpG sites in one or more genes of the cfDNA in the first cfDNA sample and the second cfDNA sample, wherein the one or more genes are selected from the group consisting of AK055957, APC, GSTP1, HOXA1, PFKP, PRDM2, RUNX3, SEPTIN9, SPINT2, and WIF1, wherein detection of increased frequency of methylation of the CpG sites in the one or more genes selected from the group consisting of AK055957, APC, GSTP1, HOXA1, PFKP, PRDM2, RUNX3, SEPTIN9, SPINT2, and WIF1 in the second cfDNA sample compared to the first cfDNA sample indicate that the HCC is progressing, and detection of decreased frequency of methylation of the CpG sites in the one or more genes selected from the group consisting of AK055957, APC, GSTP1, HOXA1, PFKP, PRDM2, RUNX3, SEPTIN9, SPINT2, and WIF1 in the second cfDNA sample compared to the first cfDNA sample indicate that the HCC is not progressing. In some embodiments, the patient is monitored over a period of time by repeatedly collecting cfDNA samples at intervals and analyzing the cfDNA to determine whether or not the HCC is progressing. HCC at any stage of progression can be monitored, including primary tumors, metastases, or recurrences.

For monitoring CRC, a first cfDNA sample can be obtained from the patient at a first time point and a second cfDNA sample can be obtained from the patient at a second time point. In some embodiments, the patient is monitored for CRC by detecting methylation at one or more CpG sites in one or more genes of the cfDNA in the first cfDNA sample and the second cfDNA sample, wherein the one or more genes are selected from the group consisting of ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C, wherein detection of increased frequency of methylation of the CpG sites in the one or more genes selected from the group consisting of ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C in the second cfDNA sample compared to the first cfDNA sample indicate that the CRC is progressing, and detection of decreased frequency of methylation of the CpG sites in the one or more genes selected from the group consisting of ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C in the second cfDNA sample compared to the first cfDNA sample indicate that the CRC is not progressing. In some embodiments, the patient is monitored over a period of time by repeatedly collecting cfDNA samples at intervals and analyzing the cfDNA to determine whether or not the CRC is progressing. CRC at any stage of progression can be monitored, including primary tumors, metastases, or recurrences.

For monitoring PRAD, a first cfDNA sample can be obtained from the patient at a first time point and a second cfDNA sample can be obtained from the patient at a second time point. In some embodiments, the patient is monitored for PRAD by detecting methylation at one or more CpG sites in one or more genes of the cfDNA in the first cfDNA sample and the second cfDNA sample, wherein the one or more genes are selected from the group consisting of APC, CD44, PYCARD, RARB, and RBP1, wherein detection of increased frequency of methylation of the CpG sites in the one or more genes selected from the group consisting of APC, CD44, PYCARD, RARB, and RBP1 in the second cfDNA sample compared to the first cfDNA sample indicate that the PRAD is progressing, and detection of decreased frequency of methylation of the CpG sites in the one or more genes selected from the group consisting of APC, CD44, PYCARD, RARB, and RBP1 in the second cfDNA sample compared to the first cfDNA sample indicate that the PRAD is not progressing. In some embodiments, the patient is monitored over a period of time by repeatedly collecting cfDNA samples at intervals and analyzing the cfDNA to determine whether or not the PRAD is progressing. PRAD at any stage of progression can be monitored, including primary tumors, metastases, or recurrences.

For monitoring LUAD, a first cfDNA sample can be obtained from the patient at a first time point and a second cfDNA sample can be obtained from the patient at a second time point. In some embodiments, the patient is monitored for LUAD by detecting methylation at one or more CpG sites in one or more genes of the cfDNA in the first cfDNA sample and the second cfDNA sample, wherein the one or more genes are selected from the group consisting of AJAP1, CDO1, HOXA7, HOXA9, PTGDR, SOX17, TAC1, UNCX, ZIC4, and ZNF781, wherein detection of increased frequency of methylation of the CpG sites in the one or more genes selected from the group consisting of AJAP1, CDO1, HOXA7, HOXA9, PTGDR, SOX17, TAC1, UNCX, ZIC4, and ZNF781 in the second cfDNA sample compared to the first cfDNA sample indicate that the LUAD is progressing, and detection of decreased frequency of methylation of the CpG sites in the one or more genes selected from the group consisting of AJAP1, CDO1, HOXA7, HOXA9, PTGDR, SOX17, TAC1, UNCX, ZIC4, and ZNF781 in the second cfDNA sample compared to the first cfDNA sample indicate that the LUAD is not progressing. In some embodiments, the patient is monitored over a period of time by repeatedly collecting cfDNA samples at intervals and analyzing the cfDNA to determine whether or not the LUAD is progressing. LUAD at any stage of progression can be monitored, including primary tumors, metastases, or recurrences.

For monitoring LUSC, a first cfDNA sample can be obtained from the patient at a first time point and a second cfDNA sample can be obtained from the patient at a second time point. In some embodiments, the patient is monitored for LUSC by detecting methylation at one or more CpG sites in one or more genes of the cfDNA in the first cfDNA sample and the second cfDNA sample, wherein the one or more genes are selected from the group consisting of AJAP1, CDO1, HOXA7, HOXA9, PTGDR, SOX17, ZFP42, ZIC4, and ZNF781, wherein detection of increased frequency of methylation of the CpG sites in the one or more AJAP1, CDO1, HOXA7, HOXA9, PTGDR, SOX17, ZFP42, ZIC4, and ZNF781 genes selected from the group consisting of in the second cfDNA sample compared to the first cfDNA sample indicate that the LUSC is progressing, and detection of decreased frequency of methylation of the CpG sites in the one or more genes selected from the group consisting of AJAP1, CDO1, HOXA7, HOXA9, PTGDR, SOX17, ZFP42, ZIC4, and ZNF781 in the second cfDNA sample compared to the first cfDNA sample indicate that the LUSC is not progressing. In some embodiments, the patient is monitored over a period of time by repeatedly collecting cfDNA samples at intervals and analyzing the cfDNA to determine whether or not the LUSC is progressing. LUSC at any stage of progression can be monitored, including primary tumors, metastases, or recurrences.

The subject methods are especially useful for diagnosing or monitoring a patient, as described herein, if the patient has an underlying condition or disease that makes the patient susceptible to developing HCC, CRC, PRAD, LUAD, or LUSC.

Exemplary conditions and diseases that increase susceptibility to HCC include, but are not limited to, liver inflammation, traumatic injury to the liver, liver cirrhosis, fatty liver disease, hepatitis (e.g., alcoholic hepatitis, non-alcoholic steatohepatitis, autoimmune hepatitis, drug-induced hepatitis, or viral hepatitis), a hepatitis A virus infection, a hepatitis B virus infection, a hepatitis C virus infection, a hepatitis D virus infection, a hepatitis E virus infection, hereditary hemochromatosis, Wilson disease, primary biliary cirrhosis, and α-1-antitrypsin deficiency.

Exemplary conditions and diseases that increase susceptibility to CRC include, but are not limited to, obesity, smoking, lack of physical activity, diet high in red meat, processed meat, or alcohol, inflammatory bowel disease (e.g., Crohn's disease and ulcerative colitis), familial adenomatous polyposis, Gardner syndrome, hereditary non-polyposis, and serrated polyposis syndrome, and a genetic predisposition to developing CRC (e.g., mutations in POLE or POLD1 genes).

Exemplary conditions and diseases that increase susceptibility to PRAD include, but are not limited to, age (over 50), family history of prostate cancer, obesity, high blood pressure, diet high in processed meat, red meat, or milk products, gonorrhea, and a genetic predisposition to developing PRAD (e.g., mutations in BRCA, HPC1, or AR genes, or TMPRSS2-ETS gene family fusion).

Exemplary conditions and diseases that increase susceptibility to LUAD include, but are not limited to, cigarette smoking, and a genetic predisposition to developing LUAD (e.g., mutations in TP53, EGFR, KRAS, KEAP1, STK11, or NF1 genes.

Exemplary conditions and diseases that increase susceptibility to LUSC include, but are not limited to, cigarette smoking and a genetic predisposition to developing LUSC (e.g., mutations in TP53, MLL2, CDKN2A, KEAP1, PTEN, NOTCHI, PIK3CA, or NFE2L2).

The subject methods may also be used for assaying pre-treatment and post-treatment cfDNA samples obtained from an individual to determine whether the individual is responsive or not responsive to a treatment. For example, a first cfDNA sample can be obtained from a subject before the subject undergoes the therapy, and a second cfDNA sample can be obtained from the subject after the subject undergoes the therapy.

In one embodiment, the efficacy of a treatment of a patient for HCC is monitored by measuring the frequency or levels of methylation at one or more CpG sites selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864, and CpG sites located within 200 nucleotides thereof, in the first cfDNA sample and the second cfDNA sample; and evaluating the efficacy of the treatment, wherein detection of increased frequency or levels of methylation at one or more CpG sites selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864, and CpG sites located within 200 nucleotides thereof, in the second cfDNA sample compared to the first cfDNA sample indicate that the HCC is progressing or not responding to the treatment, and detection of decreased frequency or levels of methylation at one or more CpG sites selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864, and CpG sites located within 200 nucleotides thereof, in the second cfDNA sample compared to the first cfDNA sample indicate that the HCC is not progressing.

In another embodiment, the efficacy of a treatment of a patient for CRC is monitored by measuring the frequency or levels of methylation at one or more CpG sites selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409, and CpG sites located within 200 nucleotides thereof, in the first cfDNA sample and the second cfDNA sample; and evaluating the efficacy of the treatment, wherein detection of increased frequency or levels of methylation at one or more CpG sites selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409, and CpG sites located within 200 nucleotides thereof, in the second cfDNA sample compared to the first cfDNA sample indicate that the CRC is progressing or not responding to the treatment, and detection of decreased frequency or levels of methylation at one or more CpG sites selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409, and CpG sites located within 200 nucleotides thereof, in the second cfDNA sample compared to the first cfDNA sample indicate that the CRC is not progressing.

In another embodiment, the efficacy of a treatment of a patient for PRAD is monitored by measuring the frequency or levels of methylation at one or more CpG sites selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124, and CpG sites located within 200 nucleotides thereof, in the first cfDNA sample and the second cfDNA sample; and evaluating the efficacy of the treatment, wherein detection of increased frequency or levels of methylation at one or more CpG sites selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124, and CpG sites located within 200 nucleotides thereof, in the second cfDNA sample compared to the first cfDNA sample indicate that the PRAD is progressing or not responding to the treatment, and detection of decreased frequency or levels of methylation at one or more CpG sites selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124, and CpG sites located within 200 nucleotides thereof, in the second cfDNA sample compared to the first cfDNA sample indicate that the PRAD is not progressing.

In another embodiment, the efficacy of a treatment of a patient for LUAD is monitored by measuring the frequency or levels of methylation at one or more CpG sites selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105, and CpG sites located within 200 nucleotides thereof, in the first cfDNA sample and the second cfDNA sample; and evaluating the efficacy of the treatment, wherein detection of increased frequency or levels of methylation at one or more CpG sites selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105, and CpG sites located within 200 nucleotides thereof, in the second cfDNA sample compared to the first cfDNA sample indicate that the LUAD is progressing or not responding to the treatment, and detection of decreased frequency or levels of methylation at one or more CpG sites selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105, and CpG sites located within 200 nucleotides thereof, in the second cfDNA sample compared to the first cfDNA sample indicate that the LUAD is not progressing.

In another embodiment, the efficacy of a treatment of a patient for LUSC is monitored by measuring the frequency or levels of methylation at one or more CpG sites selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105, and CpG sites located within 200 nucleotides thereof, in the first cfDNA sample and the second cfDNA sample; and evaluating the efficacy of the treatment, wherein detection of increased frequency or levels of methylation at one or more CpG sites selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105, and CpG sites located within 200 nucleotides thereof, in the second cfDNA sample compared to the first cfDNA sample indicate that the LUSC is progressing or not responding to the treatment, and detection of decreased frequency or levels of methylation at one or more CpG sites selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105, and CpG sites located within 200 nucleotides thereof, in the second cfDNA sample compared to the first cfDNA sample indicate that the LUSC is not progressing.

The frequency or level of methylation of a cfDNA biomarker gene in a pre-treatment sample can be referred to as a “pre-treatment value” because the first sample is isolated from the individual prior to the administration of the therapy (i.e., “pre-treatment”). The frequency or level of methylation of a cfDNA biomarker gene in the pre-treatment sample can also be referred to as a “baseline value” because this value is the value to which “post-treatment” values are compared. In some cases, the baseline value (i.e., “pre-treatment value”) is determined by determining the frequency or level of methylation of a cfDNA biomarker gene in multiple (i.e., more than one, e.g., two or more, three or more, for or more, five or more, etc.) pre-treatment samples. In some cases, the multiple pre-treatment samples are isolated from an individual at different time points in order to assess natural fluctuations in biomarker levels prior to treatment. As such, in some cases, one or more (e.g., two or more, three or more, for or more, five or more, etc.) pre-treatment samples are isolated from the individual. In some embodiments, all of the pre-treatment samples will be the same type of sample (e.g., a blood sample). In some cases, two or more pre-treatment samples are pooled prior to determining the level of the biomarker in the samples. In some cases, the frequency or level of methylation of a cfDNA biomarker gene is determined separately for two or more pre-treatment samples and a “pre-treatment value” is calculated by averaging the separate measurements.

A post-treatment sample is isolated from an individual after the administration of a therapy. Thus, the frequency or level of methylation of a cfDNA biomarker gene in a post-treatment sample can be referred to as a “post-treatment value”. In some embodiments, the frequency or level of methylation of a cfDNA biomarker gene is measured in additional post-treatment samples (e.g., a second, third, fourth, fifth, etc. post-treatment sample). Because additional post-treatment samples are isolated from the individual after the administration of a treatment, the levels of a biomarker in the additional samples can also be referred to as “post-treatment values.”

The term “responsive” as used herein means that the treatment is having the desired effect such as an anti-tumor effect. For example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. When the individual does not improve in response to the treatment, it may be desirable to seek a different therapy or treatment regime for the individual.

The determination that an individual has HCC, CRC, PRAD, LUAD, or LUSC is an active clinical application of the correlation between the frequency or level of methylation of one or more cfDNA biomarker genes and the disease. For example, “determining” requires the active step of reviewing the data, which is produced during the active assaying step(s), and resolving whether an individual does or does not have HCC, CRC, PRAD, LUAD, or LUSC, or is responding or not responding to a therapy for treatment of HCC. Additionally, in some cases, a decision is made to proceed with the current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of a therapy) is to continue. If the current course of treatment is not effective in treating HCC, CRC, PRAD, LUAD, or LUSC, the treatment may be altered. “Altering therapy” is used herein to mean “discontinuing therapy” or “changing the therapy” (e.g., changing the type of treatment, changing the particular dose and/or frequency of administration of medication, e.g., increasing the dose and/or frequency). In some cases, therapy can be altered until the individual is deemed to be responsive. In some embodiments, altering therapy means changing which type of treatment is administered, discontinuing a particular treatment altogether, etc.

As a non-limiting illustrative example, a patient may be initially treated with a chemotherapeutic agent. Then to “continue treatment” would be to continue with this type of treatment. If the current course of treatment is not effective, the treatment may be altered, e.g., increasing dosage or frequency of a treatment for HCC, CRC, PRAD, LUAD, or LUSC, changing to a different treatment, or starting palliative care for the patient. Switching treatment might involve, for example, administering a different chemotherapeutic agent or administering a different type of anti-cancer therapy such as surgery, radiation therapy, immunotherapy, etc.

In other words, the frequency or level of methylation of one or more cfDNA biomarker genes may be monitored in order to determine when to continue therapy and/or when to after therapy. As such, a post-treatment cfDNA sample can be isolated after any of the administrations and the cfDNA sample can be assayed to determine the frequency or level of methylation of one or more cfDNA biomarker genes. Accordingly, the subject methods can be used to determine whether an individual being treated for HCC, CRC, PRAD, LUAD, or LUSC is responsive or is maintaining responsiveness to a treatment.

The therapy can be administered to an individual any time after a pre-treatment cfDNA sample is isolated from the individual, but it is preferable for the therapy to be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment cfDNA sample is isolated (or, when multiple pre-treatment cfDNA samples are isolated, after the final pre-treatment cfDNA sample is isolated).

In some cases, more than one type of therapy may be administered to the individual. For example, a subject who has HCC, CRC, PRAD, LUAD, or LUSC may undergo surgical resection of a tumor followed by administration of a chemotherapeutic agent or biologic agent. Systemic therapy may be administered if the cancer spreads beyond the site of the primary tumor or undergoes metastasis.

In some embodiments, the methylated cfDNA biomarkers are used for monitoring for a recurrence of HCC, CRC, PRAD, LUAD, or LUSC in a patient. For example, a first cfDNA can be obtained from the patient after treatment for a previous occurrence of HCC, CRC, PRAD, LUAD, or LUSC at a first time point when the patient is characterized as cancer-free from imaging or other diagnostic modalities. The levels or frequency of methylation at one or more CpG sites in promoter regions of one or more biomarker genes in cfDNA from the first cfDNA sample can be measured. A second cfDNA sample can be obtained from the patient at a second time point during a period of monitoring for the recurrence. The levels or frequency of methylation at one or more CpG sites in promoter regions of one or more biomarker genes in cfDNA from the second cfDNA sample can also be measured. If the patient has a positive diagnosis for the recurrence of the HCC, CRC, PRAD, LUAD, or LUSC based on the levels or frequency of methylation of the one or more CpG sites, the patient should be treated for the recurrence of the HCC, CRC, PRAD, LUAD, or LUSC. In some embodiments, the patient is monitored for a recurrence over a period of time by repeatedly collecting cfDNA samples at intervals and analyzing the cfDNA to determine whether or not the patient has a recurrence of HCC, CRC, PRAD, LUAD, or LUSC. In some embodiments, the patient is monitored for a recurrence repeatedly over a period of 1 month, 2 months, 4 months, 6 months, 8 months, 1 year, 2 years, 3 years, 4 years, 5 years, or longer by the methods described herein.

In one embodiment, a method of monitoring for a recurrence of HCC in a patient and treating the patient for the recurrence is provided, the method comprising: a) obtaining a first blood sample from the patient after treatment for a previous occurrence of HCC at a first time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from AK055957, APC, GSTP1, HOXA1, PFKP, PRDM2, RUNX3, SEPTIN9, SPINT2, and WIF1; c) obtaining a second blood sample from the patient at a second time point during a period of monitoring for the recurrence; d) measuring levels of methylation at the one or more CpG sites in promoter regions of the one or more biomarker genes in cfDNA from the second blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA of the second blood sample compared to the cfDNA of the first blood sample indicates that the HCC has recurred; e) treating the patient for the recurrence of the HCC, if the patient has a positive diagnosis for the recurrence of the HCC based on the levels of methylation of the one or more CpG sites; and f) repeating steps c)-e) subsequently during the period of monitoring for the recurrence.

In another embodiment, a method of monitoring for a recurrence of CRC in a patient and treating the patient for the recurrence is provided, the method comprising: a) obtaining a first blood sample from the patient after treatment for a previous occurrence of CRC at a first time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from ALX4, CNRIP1, COL4A1, COL4A2, EFEMP1, EYA4, FBN1, HIC, IKZF1, MIR34C, NDRG4, SDC2, SEPTIN9, SOX21, TFP12, TMEFF2, and UNC5C; c) obtaining a second blood sample from the patient at a second time point during a period of monitoring for the recurrence; d) measuring levels of methylation at one or more CpG sites in promoter regions of one or more biomarker genes in cfDNA from the second blood sample, wherein the one or more biomarker genes are selected from ALX4, CNRIP1, COL4A1, COL4A2, EFEMP1, EYA4, FBN1, HIC, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C in the cfDNA of the second blood sample compared to the cfDNA of the first blood sample indicates that the CRC has recurred; e) treating the patient for the recurrence of the CRC, if the patient has a positive diagnosis for the recurrence of the CRC based on the levels of methylation of the one or more CpG sites; and f) repeating steps c)-e) subsequently during the period of monitoring for the recurrence.

In another embodiment, a method of monitoring for a recurrence of PRAD in a patient and treating the patient for the recurrence is provided, the method comprising: a) obtaining a first blood sample from the patient after treatment for a previous occurrence of PRAD at a first time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from APC, CD44, PYCARD, RARB, and RBP1; c) obtaining a second blood sample from the patient at a second time point during a period of monitoring for the recurrence; d) measuring levels of methylation at one or more CpG sites in promoter regions of the one or more biomarker genes in cfDNA from the second blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 in the cfDNA of the second blood sample compared to the cfDNA of the first blood sample indicates that the PRAD has recurred; e) treating the patient for the recurrence of the PRAD, if the patient has a positive diagnosis for the recurrence of the PRAD based on the levels of methylation of the one or more CpG sites; and f) repeating steps c)-e) subsequently during the period of monitoring for the recurrence.

In another embodiment, a method of monitoring for a recurrence of LUAD in a patient and treating the patient for the recurrence is provided, the method comprising: a) obtaining a first blood sample from the patient after treatment for a previous occurrence of LUAD at a time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from AJAP1, CDO1, HOXA7, HOXA9, PTGDR, SOX17, TAC1, UNCX, ZIC4, and ZNF781; c) obtaining a second blood sample from the patient during a period of monitoring for the recurrence; d) measuring levels of methylation at one or more CpG sites in promoter regions of one or more biomarker genes in cfDNA from the second blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 in the cfDNA of the second blood sample compared to the cfDNA of the first blood sample indicates that the LUAD has recurred; e) treating the patient for the recurrence of the LUAD, if the patient has a positive diagnosis for the recurrence of the LUAD based on the levels of methylation of the one or more CpG sites; and f) repeating steps c)-e) subsequently during the period of monitoring for the recurrence.

In another embodiment, a method of monitoring for a recurrence of LUSC in a patient and treating the patient for the recurrence is provided, the method comprising: a) obtaining a first blood sample from the patient after treatment for a previous occurrence of PRAD at a time point when the patient is characterized as cancer-free from imaging or other diagnostic modalities; b) measuring levels of methylation at one or more CpG sites in promoter regions of one or more biomarker genes in cell-free DNA (cfDNA) from the first blood sample, wherein the one or more biomarker genes are selected from AJAP1, CDO1, HOXA7, HOXA9, PTGDR, SOX17, ZFP42, ZIC4, and ZNF781; c) obtaining a second blood sample from the patient during a period of monitoring for the recurrence; d) measuring levels of methylation at one or more CpG sites in promoter regions of one or more biomarker genes in cfDNA from the second blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 in the cfDNA of the second blood sample compared to the cfDNA of the first blood sample indicates that the LUSC has recurred; e) treating the patient for the recurrence of the LUSC, if the patient has a positive diagnosis for the recurrence of the LUSC based on the levels of methylation of the one or more CpG sites; and f) repeating steps c)-e) subsequently during the period of monitoring for the recurrence.

In some embodiments, the subject methods include providing an analysis indicating whether the individual is determined to have HCC, CRC, PRAD, LUAD, or LUSC or a recurrence of HCC, CRC, PRAD, LUAD, or LUSC. The analysis may further provide an analysis of whether an individual is responsive or not responsive to a treatment, or whether the individual is determined to be maintaining responsiveness or not maintaining responsiveness to a treatment for HCC, CRC, PRAD, LUAD, or LUSC. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.).

Detecting Methylation of cfDNA

Any suitable method known in the art can be used for detecting methylation at CpG sites in cfDNA. Exemplary techniques for detecting methylation include, without limitation, methylation-sensitive arbitrarily-primed polymerase chain reaction (MS AP-PCR), methylation-sensitive single nucleotide primer extension (Ms-SNuPE), methylation-specific PCR (MSP), methylation-sensitive DNA restriction enzyme analysis, restriction enzyme-based sequencing, restriction enzyme-based microarray analysis, combined bisulfite restriction analysis (COBRA), methylated CpG island amplification (MCA), methylated CpG island amplification and microarray (MCAM), Hpall tiny fragment enrichment by ligation-mediated PCR (HELP), bisulfite sequencing, bisulfite microarray analysis, methylation-specific pyrosequencing, HELP-sequencing (HELP-seq), TET-assisted pyridine borane sequencing (TAPS), Glal hydrolysis and ligation adapter dependent PCR (GLAD-PCR), methylated DNA immunoprecipitation-sequencing (MeDIP-Seq), or methylated DNA immunoprecipitation-microarray analysis (MeDIP-chip), Southern blotting with methyl-sensitive restriction enzymes, and methylation-specific giant magnetoresistive sensor-based microarray analysis.

Bisulfite sequencing uses bisulfite treatment of DNA before sequencing to detect methylation sites. Treatment of DNA with bisulfite converts cytosine residues to uracil, but does not affect methylated cytosine residues. After bisulfite treatment, the only cytosines remaining in the DNA are methylated cytosines. Thus, sequencing the DNA after bisulfite treatment reveals the methylation status of individual cytosine residues at single-nucleotide resolution (Reinders et al. (2010) Epigenomics 2(2):209-20, Chatterjee et al. (2012) Nucleic Acids Research 40(10): e79, Wreczycka et al. (2017) J. Biotechnol. 261:105-115, Shafi et al. (2018) Brief Bioinform. 19(5):737-753; herein incorporated by reference).

The MS AP-PCR assay uses methylation-sensitive restriction enzymes to digest DNA and PCR with CG-rich primers to selectively amplify regions that contain CpG dinucleotides (see, e.g., Gonzalgo et al. (1997) Cancer Research 57: 594-599, herein incorporated by reference).

The MethyLight assay uses bisulfite-dependent, quantitative fluorescence-based real-time PCR with methylation-specific priming and methylation-specific fluorescent probing for detection of DNA methylation. Digital MethyLight combines the MethyLight assay with digital PCR to allow detection of individual methylated molecules (see, e.g., Eads et al. (1999) Cancer Res. 59: 2302-2306, Campan et al. (2018) Methods Mol. Biol. 1708:497-513; herein incorporated by reference).

The HeavyMethy assay uses methylation specific blocking probes (also referred to herein as blockers) covering CpG positions between, or covered by, amplification primers to enable methylation-specific selective amplification of a nucleic acid sample.

The HeavyMethyl MethyLight assay is a variation of the MethyLight™ assay, wherein the MethyLight™ assay is combined with methylation specific blocking probes covering CpG positions between the amplification primers.

The Ms-SNuPE assay uses bisulfite treatment of DNA combined with PCR using primers designed to hybridize immediately upstream of the CpG site(s) and electrophoresis of amplicons on polyacrylamide gels for visualization and quantitation. Treatment of DNA (genomic or cfDNA) with sodium bisulfite causes unmethylated cytosines to be converted to uracils, During the PCR step, uracil is replicated as thymine, and methylcytosine is replicated as cytosine during amplification. The ratio of methylated versus unmethylated cytosine (C versus T) at the original CpG sites can be determined by incubating the gel-isolated PCR product, primer(s), and Taq polymerase with either [³²P]dCTP or [³²P]TTP followed by denaturing polyacrylamide gel electrophoresis and phosphorimage analysis. Ms-SNuPE primers can also be designed to incorporate either [³²P]dATP or [³²P]dGTP into the opposite strand to assess methylation status depending on which CpG site is analyzed (see, e.g., Gonzalgo & Jones (1997) Nucleic Acids Res. 25: 2529-2531; Gonzalgo et al. (2007) Nat. Protoc. 2(8):1931-6; herein incorporated by reference).

The MSP assay uses bisulfite treatment of DNA for conversion of non-methylated cytosines to uracils and subsequent amplification with primers specific for methylated versus unmethylated DNA (see, e.g., Herman et al. (1996) Proc. Natl. Acad. Sci. USA 93: 9821-9826, and U.S. Pat. No. 5,786,146; herein incorporated by reference).

The COBRA assay uses bisulfite treatment of DNA for conversion of non-methylated cytosines to uracils, locus-specific PCR amplification of the bisulfite-converted DNA, restriction digestion, electrophoresis an analysis of restriction patterns on a gel (see, e.g., Xiong & Laird (1997) Nucleic Acids Res. 25: 2532-2534; Bilichak et al. (2017) Methods Mol. Biol. 1456:63-71; herein incorporated by reference).

The MCA assay uses methylation-sensitive restriction enzymes to digest DNA, followed by adaptor ligation and PCR to selectively amplify methylated CpG-rich sequences (see, e.g., Toyota et al. (1999) Cancer Res. 59: 2307-12, and WO 00/26401A1; herein incorporated by reference).

The MCAM assay uses MCA in combination with a CpG island microarray to detect DNA methylation in a high-throughput fashion (see, e.g., Estecio et al. (2007) Genome Res. 17(10):1529-1536; herein incorporated by reference).

The HELP assay uses the methylation-sensitive restriction enzyme, Hpall, to cut DNA, and a methylation-insensitive isoschizomer, Mspl, as a control. Microarray analysis is performed with microarrays containing probes designed to detect the Hpall/Mspl fragments. HELP-seq combines the HELP assay with massively parallel sequencing of DNA methylation sites (see, e.g., Greally (2018) Methods Mol. Biol. 1708:191-207; Suzuki et al. (2010) Methods 52(3):218-22; herein incorporated by reference).

The GLAD-PCR assay uses a site-specific methyl-directed DNA-endonucleases that cleave only methylated DNA, followed by ligation of DNA fragments to universal adapters for high-throughput PCR (see, e.g., Malyshev et al. (2020) Acta Naturae 12(3):124-133; Russian Patent RU 2525710; herein incorporated by reference).

The MeDIP assay uses an antibody against 5-methylcytosine to immunoprecipitate methylated DNA fragments. This technique can be combined with high-throughput DNA detection methods using microarray hybridization (MeDIP-chip) or next-generation sequencing (MeDIP-seq). See, e.g., Weber et al. (2005) Nat. Genet. 37 (8): 853-862, Palmke et al. (2011) Methods 53(2):175-184, Quackenbush et al. (2008) Cancer Res. 68(6):1786-1796, Zhu et al. (2019) Analyst 144(6):1904-1915, Yang et al. (2014) Life Sci. 113(1-2):45-54; herein incorporated by reference.

TET-assisted pyridine borane sequencing (TAPS) uses the ten-eleven translocation (TET) enzyme to catalyze oxidation of 5-methylcytosine and 5-hydroxymethylcytosine to 5-carboxylcytosine, followed by pyridine borane reduction to produce dihydrouracil. Unmodified cytosine is not affected. See, e.g., Liu et al. (2019) Nat Biotechnol. 37:424-429; herein incorporated by reference.

Methylation-specific giant magnetoresistive sensor-based microarray analysis combines methylation specific PCR and melt curve analysis on a giant magnetoresistive (GMR) biosensor. The GMR biosensor comprises synthetic DNA probes that target methylated or unmethylated CpG sites in the PCR amplicons. After hybridization of the PCR amplicons to the GMR biosensor, the difference in melting temperature (Tm) between the two types of probes is measured. See, e.g., Rizzi et al. (2017) ACS Nano. 11(9): 8864-8870, Nesvet et al. (2019) Biosens Bioelectron 124-125:136-142; herein incorporated by reference.

Southern Blotting can also be used to detect DNA methylation. The DNA is first digested with methylation-sensitive restriction enzymes, and the restriction fragments are analyzed by Southern Blot.

Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who do not have recurrence.

Methylation status can optionally be represented or indicated by a “methylation value” (e.g., representing a methylation frequency, fraction, ratio, percent, etc.). A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

The methylation state may be expressed in terms of a fraction or percentage of individual strands of DNA that is methylated at a particular site relative to the total population of DNA in the sample comprising that particular site. As used herein, “methylation frequency” or “methylation percent (%)” refer to the number of instances in which a molecule or locus is methylated relative to the number of instances the molecule or locus is unmethylated.

### Data Analysis

In some embodiments, one or more pattern recognition methods can be used in analyzing the data for cfDNA methylation. The quantitative values may be combined in linear or non-linear fashion to calculate one or more risk scores for HCC, CRC, PRAD, LUAD, or LUSC for an individual. In some embodiments, measurements for a methylated cfDNA biomarker or combinations of biomarkers are formulated into linear or non-linear models or algorithms (e.g., a ‘biomarker signature’) and converted into a likelihood score. This likelihood score indicates the probability that a cfDNA sample is from a patient who has no evidence of disease or a patient who has HCC, CRC, PRAD, LUAD, or LUSC. A likelihood score can also be used to distinguish among different stages of cancer progression. The models and/or algorithms can be provided in machine readable format, and may be used to correlate the frequency or levels of methylation at CpG sites in cfDNA biomarker genes or a biomarker profile with a disease state, and/or to designate a treatment modality for a patient or class of patients.

Analyzing the levels of a plurality of biomarkers may comprise the use of an algorithm or classifier. In some embodiments, a machine learning algorithm is used to classify a patient as having HCC, CRC, PRAD, LUAD, or LUSC. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Kits

Also provided are kits that can be used to detect the methylated cfDNA biomarkers described herein. Such kits can be used to diagnose a subject with HCC, CRC, PRAD, LUAD, or LUSC, detect a recurrence of HCC, CRC, PRAD, LUAD, or LUSC, therapy selection, or monitoring responses to treatment. The kit may include one or more agents for detection of methylated cfDNA biomarkers, a container for holding a biological sample comprising cfDNA (e.g., blood or plasma) isolated from a human subject suspected of having HCC, CRC, PRAD, LUAD, or LUSC; and printed instructions for reacting agents with the biological sample or a portion of the biological sample to detect the frequency or level of methylation at one or more CpG sites in cfDNA in the biological sample. The agents may be packaged in separate containers. The kit may further comprise one or more control reference samples and reagents for performing a methylation assay (e.g., bisulfite sequencing, MS AP-PCR, MethyLight™, Digital MethyLight™, HeavyMethyl™, HeavyMethyl™ MethyLight™, Ms-SNuPE, MSP, COBRA, MCA, MCAM, HELP, HELP-seq, GLAD-PCR, MeDIP-Seq, MeDIP-chip, and the like). For example, the subject kits may include agents for determining the frequency or level of methylation such as a bisulfite reagent, methylation-sensitive restriction enzymes, PCR primers that selectively amplify DNA regions that contain CpG dinucleotides, methylation-specific primers, methylation-specific probes, or a combination thereof.

For example, the kits can be used to detect methylation of one or more of the biomarkers described herein, which show increased frequency of methylation in cfDNA samples from patients who have HCC, CRC, PRAD, LUAD, or LUSC compared to healthy control subjects or subjects without cancer.

In some embodiments, a kit comprises agents for determining the frequency or level of methylation at one or more CpG sites in promoter regions of the genes: AK055957, APC, GSTP1, HOXA1, PFKP, PRDM2, RUNX3, SEPTIN9, SPINT2, and WIF1. In some embodiments, the kit comprises agents for determining the frequency or level of methylation at one or more CpG sites selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864, and CpG sites located within 200 nucleotides thereof. In some embodiments, the kit comprises agents for determining the frequency or level of methylation at the CpG sites: cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864.

In some embodiments, a kit comprises agents for determining the frequency or level of methylation at one or more CpG sites in promoter regions of the genes: ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C. In some embodiments, the kit comprises agents for determining the frequency or level of methylation at one or more CpG sites selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409, and CpG sites located within 200 nucleotides thereof. In some embodiments, the kit comprises agents for determining the frequency or level of methylation at the CpG sites: cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409.

In some embodiments, a kit comprises agents for determining the frequency or level of methylation at one or more CpG sites in promoter regions of the genes: APC, CD44, PYCARD, RARB, and RBP1. In some embodiments, the kit comprises agents for determining the frequency or level of methylation at one or more CpG sites selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124, and CpG sites located within 200 nucleotides thereof. In some embodiments, the kit comprises agents for determining the frequency or level of methylation at the CpG sites: cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124.

In some embodiments, a kit comprises agents for determining the frequency or level of methylation at one or more CpG sites in promoter regions of the genes: AJAP1, CDO1, HOXA7, HOXA9, PTGDR, SOX17, TAC1, UNCX, ZIC4, and ZNF781. In some embodiments, the kit comprises agents for determining the frequency or level of methylation at one or more CpG sites selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105, and CpG sites located within 200 nucleotides thereof. In some embodiments, the kit comprises agents for determining the frequency or level of methylation at the CpG sites: cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105.

In some embodiments, a kit comprises agents for determining the frequency or level of methylation at one or more CpG sites in promoter regions of the genes: AJAP1, CDO1, HOXA7, HOXA9, PTGDR, SOX17, ZFP42, ZIC4, and ZNF781. In some embodiments, the kit comprises agents for determining the frequency or level of methylation at one or more CpG sites selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105, and CpG sites located within 200 nucleotides thereof. In some embodiments, the kit comprises agents for determining the frequency or level of methylation at the CpG sites: cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-98 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of diagnosing and treating hepatocellular carcinoma (HCC) in a patient, the method comprising:
a) obtaining a blood sample from the patient;
b) measuring levels of methylation at one or more CpG sites in promoter regions of one or more biomarker genes in cell-free DNA (cfDNA) from the blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 compared to reference value ranges for methylation at the one or more CpG sites in the cfDNA indicate that the patient has the HCC; and
c) treating the patient for the HCC, if the patient has a positive diagnosis for the HCC based on the levels of methylation of the one or more CpG sites.

2. The method of aspect 1, wherein the one or more CpG sites are selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 and CpG sites located within 200 nucleotides thereof.

3. The method of aspect 2, wherein said measuring levels of methylation comprises measuring levels of methylation at the cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 CpG sites.

4. The method of any one of aspects 1-3, wherein said treating the patient for HCC comprises surgical resection of an HCC tumor, liver transplantation, radiofrequency ablation, cryoablation, radiation therapy, chemotherapy, immunotherapy, or biologic therapy.

5. The method of any one of aspects 1-4, wherein the reference value ranges for methylation at the one or more CpG sites are obtained from cfDNA from one or more blood samples from one or more control subjects not having HCC.

6. An in vitro method of diagnosing hepatocellular carcinoma (HCC) in a patient, the method comprising:
a) obtaining a blood sample from the patient; and
b) measuring levels of methylation at one or more CpG sites in promoter regions of one or more biomarker genes in cell-free DNA (cfDNA) from the blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 compared to reference value ranges for methylation at the one or more CpG sites in the cfDNA indicate that the patient has the HCC.

7. A method of monitoring hepatocellular carcinoma (HCC) in a patient, the method comprising:
a) obtaining a first blood sample from the patient at a first time point and a second blood sample from the patient later at a second time point; and
b) measuring levels of methylation at one or more CpG sites in promoter regions of one or more biomarker genes in cell-free DNA (cfDNA) from the first blood sample and the second blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA from the second blood sample compared to the cfDNA from the first blood sample indicate that the HCC is progressing, and detection of decreased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA from the second blood sample compared to the cfDNA from the first blood sample indicate that the HCC is not progressing.

8. The method of aspect 7, wherein the HCC is a primary tumor, a metastasis, or a recurrence.

9. The method of aspect 7 or 8, wherein the first time point is before a treatment of the patient for HCC is started and the second time point is during or after the treatment.

10. The method of aspect 9, wherein the treatment is surgical resection, liver transplantation, radiofrequency ablation, cryoablation, radiation therapy, chemotherapy, immunotherapy, or biologic therapy.

11. The method of any one of aspects 7-10, further comprising repeating steps a) and b).

12. The method of any one of aspects 7-11, further comprising increasing dosage or frequency of a treatment for HCC, changing to a different treatment, or starting palliative care for the patient if the HCC is progressing.

13. The method of any one of aspects 7-12, wherein the one or more CpG sites are selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 and CpG sites located within 200 nucleotides thereof.

14. The method of aspect 13, wherein said measuring levels of methylation comprises measuring levels of methylation at the cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 CpG sites.

15. A method of monitoring for a recurrence of hepatocellular carcinoma (HCC) in a patient and treating the patient for the recurrence, the method comprising:
a) obtaining a first blood sample from the patient after treatment for a previous occurrence of HCC at a first time point when the patient is characterized as cancer-free from imaging or other diagnostic modalities;
b) measuring levels of methylation at one or more CpG sites in promoter regions of one or more biomarker genes in cell-free DNA (cfDNA) from the first blood sample, wherein the one or more biomarker genes are selected from AK055957, APC, GSTP1, HOXA1, PFKP, PRDM2, RUNX3, SEPTIN9, SPINT2, and WIF1;
c) obtaining a second blood sample from the patient at a second time point during a period of monitoring for the recurrence;
d) measuring levels of methylation at one or more CpG sites in promoter regions of one or more biomarker genes in cfDNA from the second blood sample, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA of the second blood sample compared to the cfDNA of the first blood sample indicates that the HCC has recurred;
e) treating the patient for the recurrence of the HCC, if the patient has a positive diagnosis for the recurrence of the HCC based on the levels of methylation of the one or more CpG sites; and
f) repeating steps c)-e) subsequently during the period of monitoring for the recurrence.

16. The method of aspect 15, wherein the one or more CpG sites are selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864, and CpG sites located within 200 nucleotides thereof.

17. The method of aspect 16, wherein said measuring levels of methylation comprises measuring levels of methylation of the cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 CpG sites.

18. The method of any one of aspects 15-17, wherein said treating the patient for the recurrence of HCC comprises surgical resection of an HCC tumor, liver transplantation, radiofrequency ablation, cryoablation, radiation therapy, chemotherapy, immunotherapy, or biologic therapy.

19. The method of any one of aspects 1-18, further comprising measuring levels of alpha-fetoprotein (AFP).

20. A cell-free DNA methylated at one or more CpG sites selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864, and CpG sites located within 200 nucleotides thereof, for use as a biomarker for diagnosis of hepatocellular carcinoma (HCC), detecting recurrence of HCC, or monitoring treatment of HCC.

21. A method of diagnosing and treating colorectal adenocarcinoma (CRC) in a patient, the method comprising:
a) obtaining a blood sample from the patient;
b) measuring levels of methylation at one or more CpG sites in promoter regions of one or more biomarker genes in cell-free DNA (cfDNA) from the blood sample, wherein the one or more biomarker genes are selected from ALX4, CNRIP1, COL4A1, COL4A2, EFEMP1, EYA4, FBN1, HIC, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC, IKZF1, MIR34C, NDRG4, SDC2, SEPTIN9, SOX21, TFP12, TMEFF2, and UNC5C compared to reference value ranges for methylation at the one or more CpG sites in cfDNA indicate that the patient has the CRC; and
c) treating the patient for the CRC, if the patient has a positive diagnosis for the CRC based on the levels of methylation of the one or more CpG sites.

22. The method of aspect 21, wherein the one or more CpG sites are selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409, and CpG sites located within 200 nucleotides thereof.

23. The method of aspect 22, wherein said measuring levels of methylation comprises measuring levels of methylation of the cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409 CpG sites.

24. The method of any one of aspects 21-23, wherein said treating the patient for CRC comprises surgical resection of a CRC tumor, radiation therapy, chemotherapy, immunotherapy, or biologic therapy.

25. The method of any one of aspects 21-24, wherein the reference value ranges for methylation at the one or more CpG sites are obtained from cfDNA from one or more blood samples from one or more control subjects not having CRC.

26. A cell-free DNA methylated at one or more CpG selected form cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409, and CpG sites located within 200 nucleotides thereof, for use as a biomarker for diagnosis of colorectal adenocarcinoma (CRC), detecting recurrence of CRC, or monitoring treatment of CRC.

27. A method of monitoring colorectal adenocarcinoma (CRC) in a patient, the method comprising:
a) obtaining a first blood sample from the patient at a first time point and a second blood sample from the patient later at a second time point; and
b) measuring levels of methylation at one or more CpG sites in promoter regions of one or more biomarker genes in cell-free DNA (cfDNA) from the first blood sample and the second blood sample, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C in the cfDNA from the second blood sample compared to the cfDNA from the first blood sample indicate that the CRC is progressing, and detection of decreased levels of methylation at the one or more CpG sites in the promoter regions of the one or more biomarker genes selected from ALX4, CNRIP1, COL4A1, COL4A2, EFEMP1, EYA4, FBN1, HIC, IKZF1, MIR34C, NDRG4, SDC2, SEPTIN9, SOX21, TFP12, TMEFF2, and UNC5C in the cfDNA from the second blood sample compared to the cfDNA from the first blood sample indicate that the CRC is not progressing.

28. The method of aspect 27, wherein the CRC is a primary tumor, a metastasis, or a recurrence.

29. The method of aspect 27 or 28, wherein the first time point is before a treatment of the patient for CRC is started and the second time point is during or after the treatment.

30. The method of aspect 29, wherein the treatment is surgical resection of a CRC tumor, radiation therapy, chemotherapy, immunotherapy, or biologic therapy.

31. The method of any one of aspects 27-30, further comprising increasing dosage or frequency of a treatment for CRC, changing to a different treatment for CRC, or starting palliative care for the patient if the CRC is progressing.

32. The method of any one of aspects 27-31, further comprising repeating steps a) and b).

33. The method of any one of aspects 27-32, wherein the one or more CpG sites are selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409 and CpG sites located within 200 nucleotides thereof.

34. The method of aspect 33, wherein said measuring levels of methylation comprises measuring levels of methylation at the cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409 CpG sites.

35. A method of monitoring for a recurrence of colorectal adenocarcinoma (CRC) in a patient and treating the patient for the recurrence, the method comprising:
a) obtaining a first blood sample from the patient after treatment for a previous occurrence of CRC at a first time point when the patient is characterized as cancer-free from imaging or other diagnostic modalities;
b) measuring levels of methylation at one or more CpG sites in promoter regions of one or more biomarker genes in cell-free DNA (cfDNA) from the first blood sample, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C;
c) obtaining a second blood sample from the patient at a second time point during a period of monitoring for the recurrence;
d) measuring levels of methylation at one or more CpG sites in promoter regions of one or more biomarker genes in cfDNA from the second blood sample, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C in the cfDNA of the second blood sample compared to the cfDNA of the first blood sample indicates that the CRC has recurred;
e) treating the patient for the recurrence of the CRC, if the patient has a positive diagnosis for the recurrence of the CRC based on the levels of methylation of the one or more CpG sites; and
f) repeating steps c)-e) subsequently during the period of monitoring for the recurrence.

36. The method of aspect 35, wherein the one or more CpG sites are selected from cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409, and CpG sites located within 200 nucleotides thereof.

37. The method of aspect 36, wherein said measuring levels of methylation comprises measuring levels of methylation of the cg14410064, cg10908369, cg25530246, cg21938148, cg03817671, cg25046074, cg08712932, cg11664500, cg12152919, cg14017655, cg14287112, cg16347317, cg20330472, cg20611276, cg22871668, cg10480343, cg22535307, cg25520679, cg07589773, cg16697214, cg18607529, cg18810347, cg20078466, cg27633530, cg01192900, cg24041078, cg06650115, cg13096260, cg18719750, cg24732574, cg25070637, cg02884239, cg06848185, cg15044248, cg19554255, cg00800383, cg19063972, cg19184934, cg27442308, cg22441533, cg01808545, cg01194057, cg07234102, cg12584684, cg15701178, and cg21782409 CpG sites.

38. The method of any one of aspects 35-37, wherein said treating the patient for the recurrence of CRC comprises surgical resection of a CRC tumor, radiation therapy, chemotherapy, immunotherapy, or biologic therapy.

39. An in vitro method of diagnosing colorectal adenocarcinoma (CRC) in a patient, the method comprising:
a) obtaining a blood sample from the patient; and
b) measuring levels of methylation at one or more CpG sites in promoter regions of one or more biomarker genes in cell-free DNA (cfDNA) from the blood sample, wherein the one or more biomarker genes are selected from ALX4, CNRIP1, COL4A1, COL4A2, EFEMP1, EYA4, FBN1, HIC, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC, IKZF1, MIR34C, NDRG4, SDC2, SEPTIN9, SOX21, TFP12, TMEFF2, and UNC5C compared to reference value ranges for methylation at the one or more CpG sites in cfDNA indicate that the patient has the CRC.

40. A method of diagnosing and treating prostate adenocarcinoma (PRAD) in a patient, the method comprising:
a) obtaining a blood sample from the patient;
b) measuring levels of methylation at one or more CpG sites in promoter regions of one or more biomarker genes in cell-free DNA (cfDNA) from the blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 compared to reference value ranges for methylation at the one or more CpG sites in cfDNA indicate that the patient has the PRAD; and
c) treating the patient for the PRAD, if the patient has a positive diagnosis for the PRAD based on the levels of methylation of the one or more CpG sites.

41. The method of aspect 40, wherein the one or more CpGs are selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124, and CpG sites located within 200 nucleotides thereof.

42. The method of aspect 41, wherein said measuring levels of methylation comprises measuring levels of methylation of the cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124 CpG sites.

43. The method of any one of aspects 40-42, wherein said treating the patient for PRAD comprises radical prostatectomy, hormonal therapy, radiation therapy, brachytherapy, cryosurgery, high-intensity focused ultrasound, chemotherapy, immunotherapy, or biologic therapy.

44. The method of any one of aspects 40-43, wherein the reference value ranges for methylation at the one or more CpG sites are obtained from cfDNA from one or more blood samples from one or more control subjects not having PRAD.

45. A cell-free DNA methylated at one or more CpG sites selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124, and CpG sites located within 200 nucleotides thereof, for use as a biomarker for diagnosis of prostate adenocarcinoma (PRAD), detecting recurrence of PRAD, or monitoring treatment of PRAD.

46. An in vitro method of diagnosing prostate adenocarcinoma (PRAD) in a patient, the method comprising:
a) obtaining a blood sample from the patient; and
b) measuring levels of methylation at one or more CpG sites in promoter regions of one or more biomarker genes in cell-free DNA (cfDNA) from the blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 compared to reference value ranges for methylation at the one or more CpG sites in cfDNA indicate that the patient has the PRAD.

47. A method of monitoring prostate adenocarcinoma (PRAD) in a patient, the method comprising:
a) obtaining a first blood sample from the patient at a first time point and a second blood sample from the patient later at a second time point; and
b) measuring levels of methylation at one or more CpG sites in promoter regions of one or more biomarker genes in circulating free DNA (cfDNA) from the first blood sample and the second blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 in the cfDNA of the second blood sample compared to the cfDNA of the first blood sample indicate that the PRAD is progressing, and detection of decreased levels of methylation at the one or more CpG sites in the promoter regions of the one or more biomarker genes selected from APC, CD44, PYCARD, RARB, and RBP1 in the cfDNA of the second blood sample compared to the cfDNA of the first blood sample indicate that the PRAD is not progressing.

48. The method of aspect 47, wherein the PRAD is a primary tumor, a metastasis, or a recurrence.

49. The method of aspect 47 or 48, wherein the first time point is before a treatment of the patient for PRAD is started and the second time point is during or after the treatment.

50. The method of any one of aspect 49, wherein the treatment is radical prostatectomy, hormonal therapy, radiation therapy, brachytherapy, cryosurgery, high-intensity focused ultrasound, chemotherapy, immunotherapy, or biologic therapy.

51. The method of any one of aspect 47-50, further comprising repeating steps a) and b).

52. The method of any one of aspects 47-51, further comprising increasing dosage or frequency of a treatment for PRAD, changing to a different treatment for PRAD, or starting palliative care for the patient if the PRAD is progressing.

53. The method of any one of aspects 47-52, wherein the one or more CpG sites are selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124, and CpG sites located within 200 nucleotides thereof.

54. The method of aspect 53, wherein said measuring levels of methylation comprises measuring levels of methylation at the cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124 CpG sites.

55. A method of monitoring for a recurrence of prostate adenocarcinoma (PRAD) in a patient and treating the patient for the recurrence, the method comprising:
a) obtaining a first blood sample from the patient after treatment for a previous occurrence of PRAD at a first time point when the patient is characterized as cancer-free from imaging or other diagnostic modalities;
b) measuring levels of methylation at one or more CpG sites in promoter regions of one or more biomarker genes in cell-free DNA (cfDNA) from the first blood sample, wherein the one or more biomarker genes are selected from APC, CD44, PYCARD, RARB, and RBP1;
c) obtaining a second blood sample from the patient at a second time point during a period of monitoring for the recurrence;
d) measuring levels of methylation at one or more CpG sites in promoter regions of one or more biomarker genes in cfDNA from the second blood sample, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 in the cfDNA of the second blood sample compared to the cfDNA of the first blood sample indicates that the PRAD has recurred;
e) treating the patient for the recurrence of the PRAD, if the patient has a positive diagnosis for the recurrence of the PRAD based on the levels of methylation of the one or more CpG sites; and
f) repeating steps c)-e) subsequently during the period of monitoring for the recurrence.

56. The method of aspect 55, wherein the one or more CpG sites are selected from cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124, and CpG sites located within 200 nucleotides thereof.

57. The method of aspect 56, wherein said measuring levels of methylation comprises measuring levels of methylation of the cg00577935, cg08571859, cg11613015, cg14479889, cg14511739, cg16970232, cg22035501, cg23938220, cg08530414, cg05214748, cg05907835, cg15468095, cg02499249, cg06720425, cg12479047, cg18094781, cg20899354, cg26124016, and cg15229124 CpG sites.

58. The method of any one of aspects 55-57, wherein said treating the patient for the recurrence of PRAD comprises radical prostatectomy, hormonal therapy, radiation therapy, brachytherapy, cryosurgery, high-intensity focused ultrasound, chemotherapy, immunotherapy, or biologic therapy.

59. The method of any one of aspects 40-44 and 46-58, further comprising measuring levels of prostate-specific antigen (PSA).

60. A method of diagnosing and treating lung adenocarcinoma (LUAD) in a patient, the method comprising:
a) obtaining a blood sample from the patient;
b) measuring levels of methylation at one or more CpG sites in promoter regions of one or more biomarker genes in cell-free DNA (cfDNA) from the blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 compared to reference value ranges for methylation at the one or more CpG sites in cfDNA indicate that the patient has the LUAD; and
c) treating the patient for the LUAD, if the patient has a positive diagnosis for the LUAD based on the levels of methylation of the one or more CpG sites.

61. The method of aspect 60, wherein the one or more CpG sites are selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105, and CpG sites located within 200 nucleotides thereof.

62. The method of aspect 61, wherein said measuring levels of methylation comprises measuring levels of methylation of the cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105 CpG sites.

63. The method of any one of aspects 60-62, wherein said treating the patient for LUAD comprises pneumonectomy, lobectomy, radiation therapy, chemotherapy, immunotherapy, or biologic therapy.

64. The method of any one of aspects 60-63, wherein the reference value ranges for methylation at the one or more CpG sites are obtained from cfDNA from one or more blood samples from one or more control subjects not having LUAD.

65. A cell-free DNA methylated at one or more CpG sites selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105, and CpG sites located within 200 nucleotides thereof, for use as a biomarker for diagnosis of lung adenocarcinoma (LUAD), detecting recurrence of LUAD, or monitoring treatment of LUAD.

66. An in vitro method of diagnosing lung adenocarcinoma (LUAD) in a patient, the method comprising:
a) obtaining a blood sample from the patient; and
b) measuring levels of methylation at one or more CpG sites in promoter regions of one or more biomarker genes in cell-free DNA (cfDNA) from the blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 compared to reference value ranges for methylation at the one or more CpG sites in cfDNA indicate that the patient has the LUAD.

67. A method of monitoring lung adenocarcinoma (LUAD) in a patient, the method comprising:
a) obtaining a first blood sample from the patient at a first time point and a second blood sample from the patient later at a second time point; and
b) measuring levels of methylation at one or more CpG sites in promoter regions of one or more biomarker genes in cell-free DNA (cfDNA) from the first blood sample and the second blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 in the cfDNA from the second blood sample compared to the cfDNA from the first blood sample indicate that the LUAD is progressing, and detection of decreased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 in the cfDNA from the second blood sample compared to the cfDNA from the first blood sample indicate that the LUAD is not progressing.

68. The method of aspect 67, wherein the LUAD is a primary tumor, a metastasis, or a recurrence.

69. The method of aspect 67 or 68, wherein the first time point is before a treatment of the patient for LUAD is started and the second time point is during or after the treatment.

70. The method of aspect 69, wherein the treatment is pneumonectomy, lobectomy, radiation therapy, chemotherapy, immunotherapy, or biologic therapy.

71. The method of any one of aspect 67-70, further comprising repeating steps a) and b).

72. The method of any one of aspects 67-71, further comprising increasing dosage or frequency of a treatment for LUAD, changing to a different treatment for LUAD, or starting palliative care for the patient if the LUAD is progressing.

73. The method of any one of aspects 67-72, wherein the one or more CpG sites are selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105, and CpG sites located within 200 nucleotides thereof.

74. The method of aspect 73, wherein said measuring levels of methylation comprises measuring levels of methylation at the cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105 CpG sites.

75. A method of monitoring for a recurrence of lung adenocarcinoma (LUAD) in a patient and treating the patient for the recurrence, the method comprising:
a) obtaining a first blood sample from the patient after treatment for a previous occurrence of LUAD at a time point when the patient is characterized as cancer-free from imaging or other diagnostic modalities;
b) measuring levels of methylation at one or more CpG sites in promoter regions of one or more biomarker genes in cell-free DNA (cfDNA) from the first blood sample, wherein the one or more biomarker genes are selected from AJAP1, CDO1, HOXA7, HOXA9, PTGDR, SOX17, TAC1, UNCX, ZIC4, and ZNF781;
c) obtaining a second blood sample from the patient during a period of monitoring for the recurrence;
d) measuring levels of methylation at one or more CpG sites in promoter regions of one or more biomarker genes in cfDNA from the second blood sample, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 in the cfDNA of the second blood sample compared to the cfDNA of the first blood sample indicates that the LUAD has recurred;
e) treating the patient for the recurrence of the LUAD, if the patient has a positive diagnosis for the recurrence of the LUAD based on the levels of methylation of the one or more CpG sites; and
f) repeating steps c)-e) subsequently during the period of monitoring for the recurrence.

76. The method of aspect 75, wherein the one or more CpG sites are selected from cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105, and CpG sites located within 200 nucleotides thereof.

77. The method of aspect 76, wherein said measuring levels of methylation comprises measuring levels of methylation of the cg11835068, cg13495205, cg17741689, cg02792792, cg08516516, cg11036833, cg14470895, cg16707405, cg23180938, cg05471296, cg05743885, cg09017619, cg15797110, cg19153763, cg20293594, cg02643054, cg05065989, cg12600174, cg18447772, cg20741169, cg22055728, cg26365299, cg02191312, cg17929687, cg04672706, cg24891539, cg24928391, cg02539083, cg05157140, cg08507422, cg20870512, cg21127068, cg24686610, cg12388007, cg03611452, cg14587524, and cg25324105 CpG sites.

78. The method of any one of aspects 75-77, wherein said treating the patient for the recurrence of LUAD comprises pneumonectomy, lobectomy, radiation therapy, chemotherapy, immunotherapy, or biologic therapy.

79. A method of diagnosing and treating lung squamous cell carcinoma (LUSC) in a patient, the method comprising:
a) obtaining a blood sample from the patient;
b) measuring levels of methylation at one or more CpG sites in promoter regions of one or more biomarker genes in cell-free DNA (cfDNA) from the blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 compared to reference value ranges for methylation at the one or more CpG sites in cfDNA indicate that the patient has the LUSC; and
c) treating the patient for the LUSC, if the patient has a positive diagnosis for the LUSC based on the levels of methylation of the one or more CpG sites.

80. The method of aspect 79, wherein the one or more CpGs are selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105, and CpG sites located within 200 nucleotides thereof.

81. The method of aspect 80, wherein said measuring levels of methylation comprises measuring levels of methylation at the cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105 CpG sites.

82. The method of any one of aspects 79-81, wherein said treating the patient for LUSC comprises pneumonectomy, lobectomy, radiation therapy, chemotherapy, immunotherapy, or biologic therapy.

83. The method of any one of aspects 79-82, wherein the reference value ranges for methylation at the one or more CpG sites are obtained from cfDNA from one or more blood samples from one or more control subjects not having LUSC.

84. A cell-free DNA methylated at one or more CpG sites selected from the CpGs within the 200 nucleotides flanking the CpGs located at cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105, and CpG sites located within 200 nucleotides thereof, for use as a biomarker for diagnosis of lung squamous cell carcinoma (LUSC), detecting recurrence of LUSC, or monitoring treatment of LUSC.

85. An in vitro method of diagnosing lung squamous cell carcinoma (LUSC) in a patient, the method comprising:
a) obtaining a blood sample from the patient; and
b) measuring levels of methylation at one or more CpG sites in promoter regions of one or more biomarker genes in cell-free DNA (cfDNA) from the blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 compared to reference value ranges for methylation at the one or more CpG sites in cfDNA indicate that the patient has the LUSC.

86. A method of monitoring lung squamous cell carcinoma (LUSC) in a patient, the method comprising:
a) obtaining a first blood sample from the patient at a first time point and a second blood sample from the patient later at a second time point; and
b) measuring levels of methylation at one or more CpG sites in promoter regions of one or more biomarker genes in circulating free DNA (cfDNA) from the first blood sample and the second blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 in the cfDNA from the second blood sample compared to the cfDNA from the first blood sample indicate that the LUSC is progressing, and detection of decreased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 in the cfDNA from the second blood sample compared to the cfDNA from the first blood sample indicate that the LUSC is not progressing.

87. The method of aspect 86, wherein the LUSC is a primary tumor, a metastasis, or a recurrence.

88. The method of aspect 86 or 87, wherein the first time point is before a treatment of the patient for LUSC is started and the second time point is during or after the treatment.

89. The method of aspect 88, wherein the treatment is pneumonectomy, lobectomy, radiation therapy, chemotherapy, immunotherapy, or biologic therapy.

90. The method of any one of aspect 86-89, further comprising repeating steps a) and b).

91. The method of any one of aspects 86-90, further comprising increasing dosage or frequency of a treatment for LUSC, changing to a different treatment for LUSC, or starting palliative care for the patient if the LUSC is progressing.

92. The method of any one of aspects 86-91, wherein the one or more CpG sites are selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105 and CpG sites located within 200 nucleotides thereof.

93. The method of aspect 92, wherein said measuring levels of methylation comprises measuring levels of methylation at the cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105 CpG sites.

94. A method of monitoring for a recurrence of prostate adenocarcinoma (LUSC) in a patient and treating the patient for the recurrence, the method comprising:
a) obtaining a first blood sample from the patient after treatment for a previous occurrence of PRAD at a time point when the patient is characterized as cancer-free from imaging or other diagnostic modalities;
b) measuring levels of methylation at one or more CpG sites in promoter regions of one or more biomarker genes in cell-free DNA (cfDNA) from the first blood sample, wherein the one or more biomarker genes are selected from AJAP1, CDO1, HOXA7, HOXA9, PTGDR, SOX17, ZFP42, ZIC4, and ZNF781;
c) obtaining a second blood sample from the patient during a period of monitoring for the recurrence;
d) measuring levels of methylation at one or more CpG sites in promoter regions of one or more biomarker genes in cfDNA from the second blood sample, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 in the cfDNA of the second blood sample compared to the cfDNA of the first blood sample indicates that the LUSC has recurred;
e) treating the patient for the recurrence of the LUSC, if the patient has a positive diagnosis for the recurrence of the LUSC based on the levels of methylation of the one or more CpG sites; and
f) repeating steps c)-e) subsequently during the period of monitoring for the recurrence.

95. The method of aspect 94, wherein the one or more CpG sites are selected from cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105, and CpG sites located within 200 nucleotides thereof.

96. The method of aspect 95, wherein said measuring levels of methylation comprises measuring levels of methylation of the cg13495205, cg02792792, cg08516516, cg11036833, cg05471296, cg05743885, cg09017619, cg10474350, cg15797110, cg19153763, cg20293594, cg02643054, cg05065989, cg12600174, cg16104915, cg18447772, cg20741169, cg22055728, cg02191312, cg17929687, cg24928391, cg24896649, cg05548555, cg06369327, cg21127068, cg03355998, cg12388007, cg03611452, cg14587524, and cg25324105 CpG sites.

97. The method of any one of aspects 94-96, wherein said treating the patient for the recurrence of LUSC comprises pneumonectomy, lobectomy, radiation therapy, chemotherapy, immunotherapy, or biologic therapy.

98. The method of any one of aspects 1-19, 21-25, 27-44, 46-64, 66-83, and 85-97, wherein said measuring the levels of methylation comprises performing methylation-specific polymerase chain reaction (PCR), quantitative methylation-specific PCR, methylation-sensitive DNA restriction enzyme analysis, methylation-specific pyrosequencing, restriction enzyme-based sequencing, restriction enzyme-based microarray analysis, TET-assisted pyridine borane sequencing (TAPS), methylated DNA immunoprecipitation-based sequencing, methylated DNA immunoprecipitation-based microarray analysis, bisulfite sequencing, bisulfite microarray analysis, or methylation-specific giant magnetoresistive sensor-based microarray analysis.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Layered Analysis of Methylated Biomarkers in Big Data for Blood-Based Multi-Cancer Diagnosis and Prognosis

### Introduction

Recent studies on methylated cfDNA have found cancer biomarkers by using microarrays and next-generation bisulfite sequencing to quantify CpG methylation frequencies in tissues and cfDNA and then comparing these frequencies between cancer patients and healthy controls (7-12). Due to the high-throughput nature of the technologies used in these approaches, they produce data with significant imbalance between the number of patients in the biomarker discovery cohort (˜50 to ˜3K) and the number of biomarkers screened in the patients (˜450K to ˜28M)—a phenomenon known as the curse of dimensionality. Statistical biomarker selection methods used on discovery cohort data in these studies pose a risk of ranking correlative biomarkers with undefined epigenetic relationships in a cancer type above biomarkers with known epigenetic roles. As a result, causative biomarkers with reproducible predictive power between patient cohorts can be filtered out during the discovery process (20-22). We hypothesized that using a knowledge-based biomarker selection method to reduce the dimensionality of methylation microarray datasets followed by the individual screening of each remaining biomarker in these datasets would identify causative biomarkers that collectively embody a tumor-specific methylation signature in cfDNA. Knowledge-based methods like Gene Set Enrichment Analysis integrate established biological relationships identified through past experiments with separate high-dimensional datasets and have been used to discover common molecular pathways in cancer (23-25).

To test this hypothesis, we created a computational, knowledge-based biomarker discovery method, Layered Analysis of Methylated Biomarkers (LAMB), that uses a meta-analysis to select an enriched set of biomarker candidates in high-dimensional CpG microarray data and filter these biomarkers against tissue methylation data from hundreds of cancer patients and blood methylation data from more than a thousand healthy patients. Here, we introduce LAMB and use it to discover sensitive cfDNA biomarker panels that: (i) detect hepatocellular carcinoma (HCC) tumors in at-risk cirrhosis patients, (ii) detect colorectal adenocarcinoma (CRC) tumors in average-risk patients, (iii) detect non-small cell lung cancer in average-risk patients, and (iv) differentiate prostate adenocarcinoma (PRAD) patients by their tumor response to therapy. Furthermore, we validate the clinical performance of the biomarker panels in independent cfDNA datasets using unbiased geometric mean scores.

### Results

### Layered Analysis of Methylated Biomarkers (LAMB) for Liver, Colorectal, Prostate, and Lung Cancer

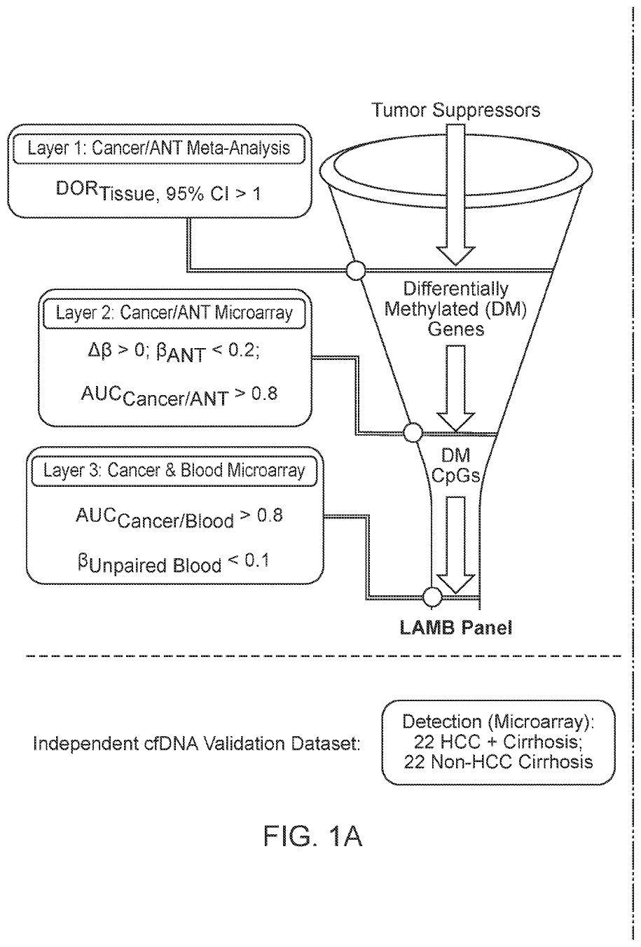

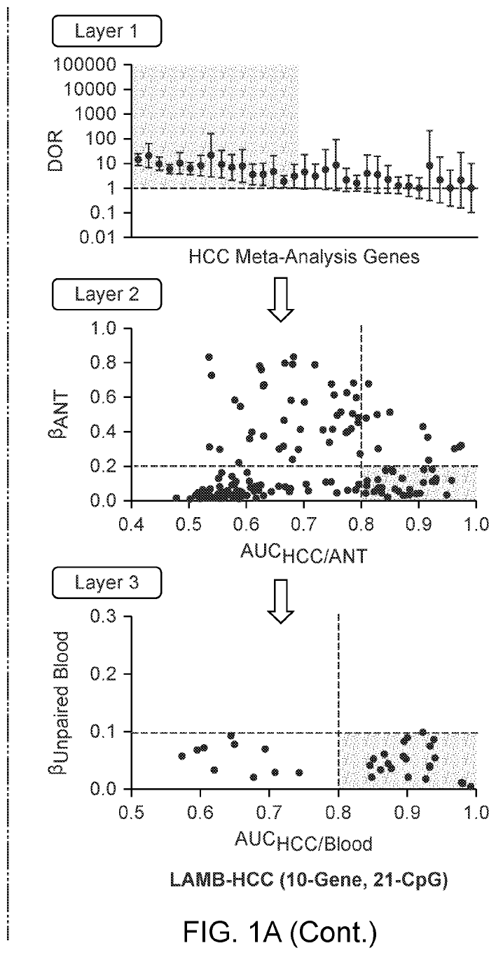

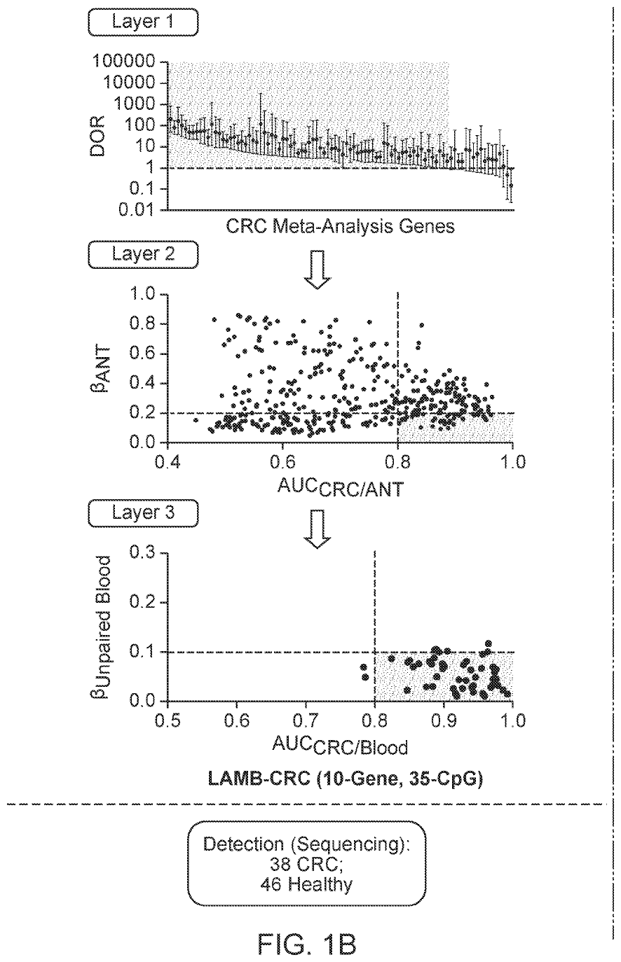

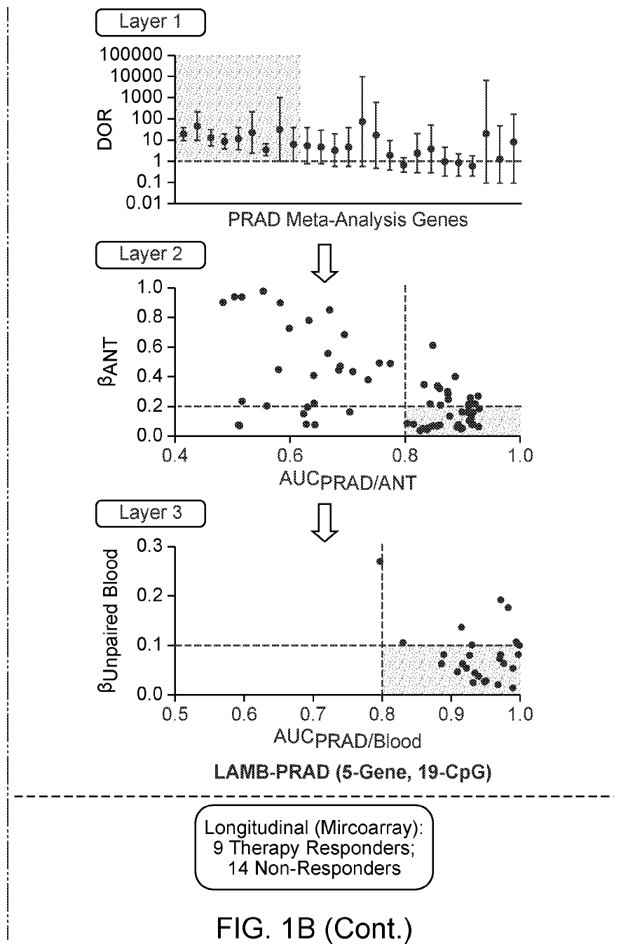

LAMB is comprised of three selection layers (FIG. 1A). Candidate tumor suppressors for a cancer type are identified in Layer 1 through a meta-analysis of methylation count data for paired malignant tissues and adjacent noncancerous tissues (ANTs) from published studies and area under the curve (AUC) values from receiver operating characteristic (ROC) curves for cfDNA from both cancer and control patients. Public Illumina HumanMethylation450 (450K) microarray datasets of paired malignant tissues and ANTs from The Cancer Genome Atlas (TCGA) and Gene Expression Omnibus (GEO) repositories are used in Layer 2 to screen CpGs in the promoter regions of genes identified in Layer 1. The remaining CpG sites are differentially methylated between cancer tissues and ANTs. These sites are then screened with public 450K data for separate malignant tissues and 1722 lysed whole blood samples from healthy patients in Layer 3 for CpGs that are differentially methylated between malignant tissue and blood. LAMB biomarker panels are comprised of CpGs that survived filtering from all three LAMB layers. Additional details on filtering criteria and data preparation for LAMB are provided in FIG. 1A and the Methods section.

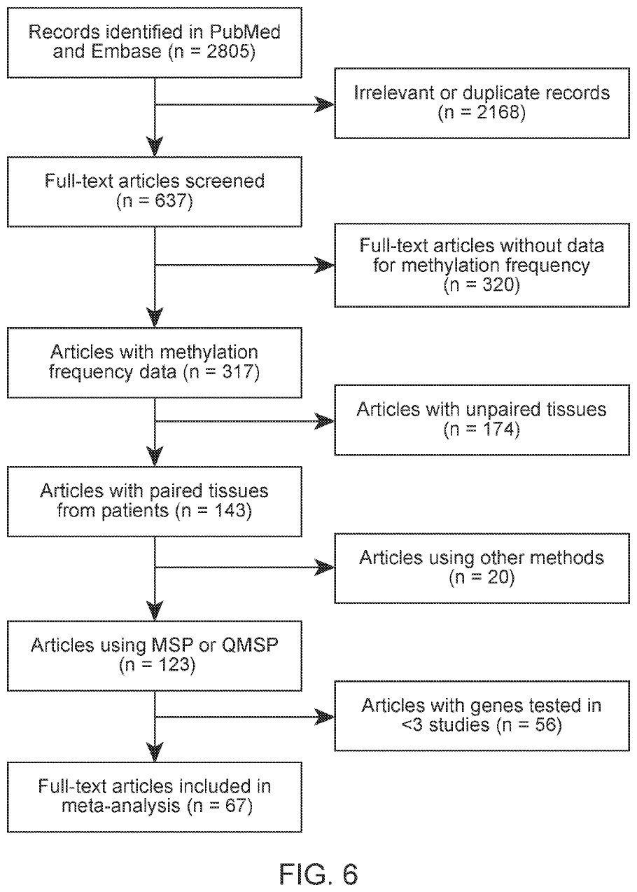

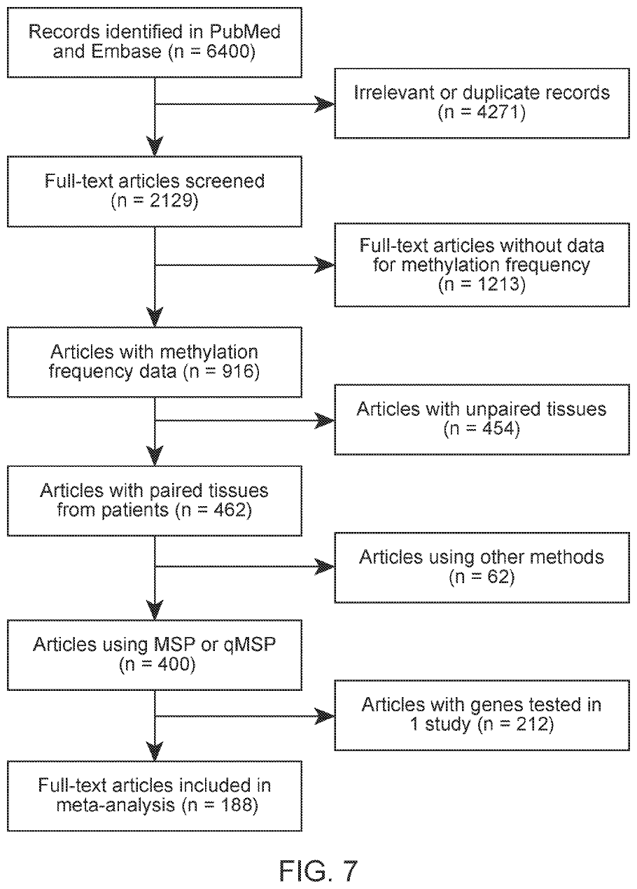

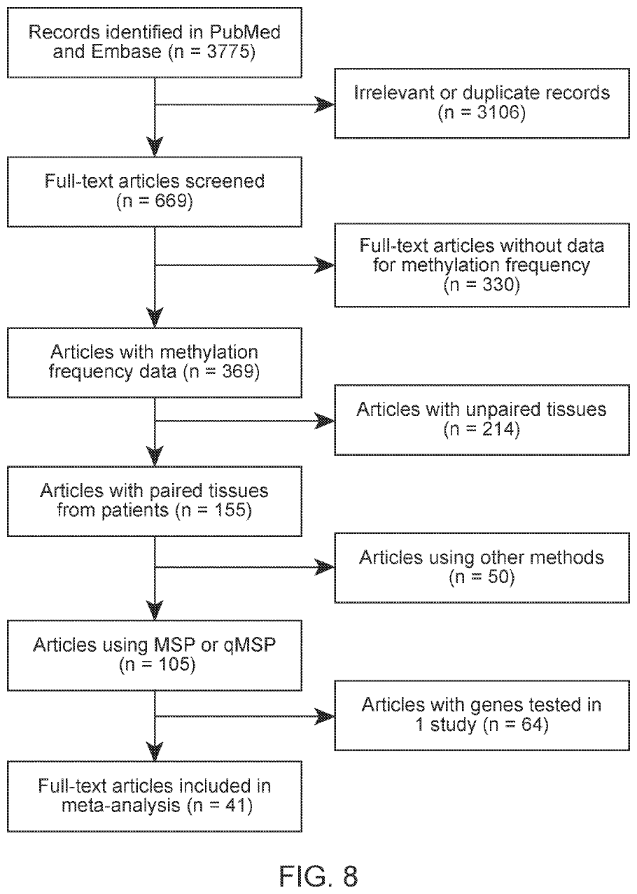

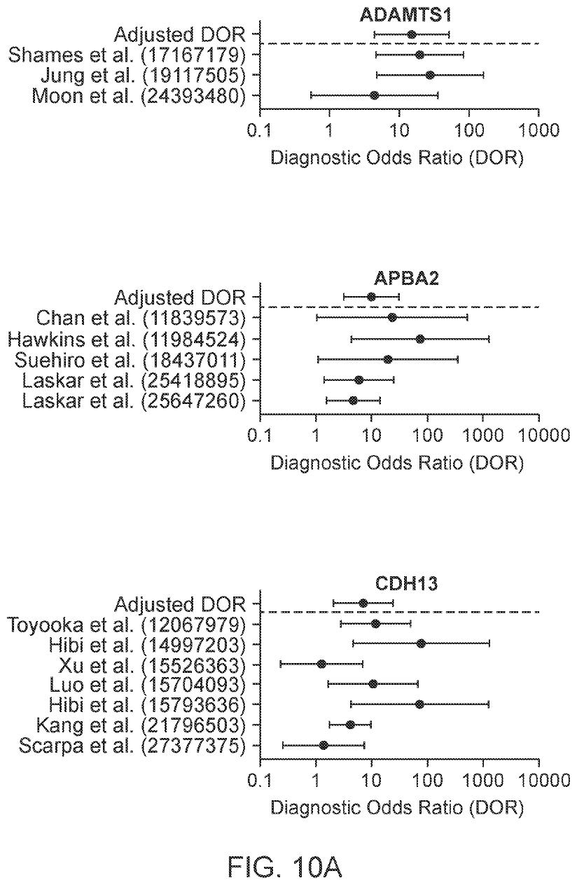

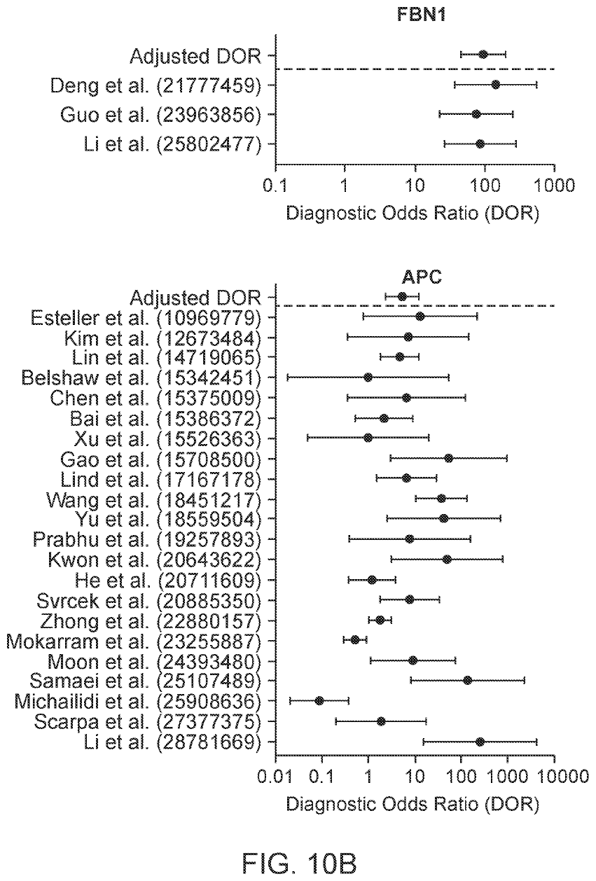

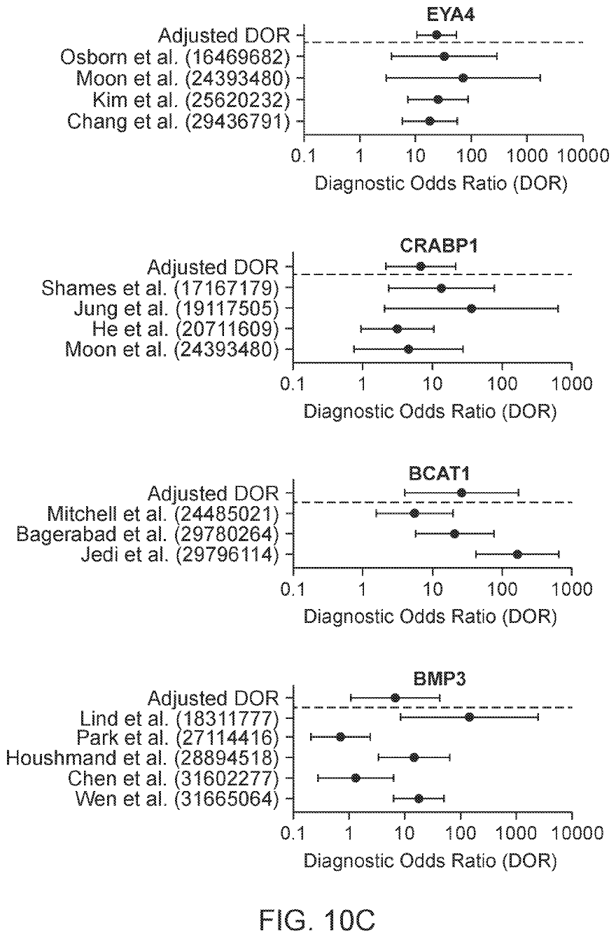

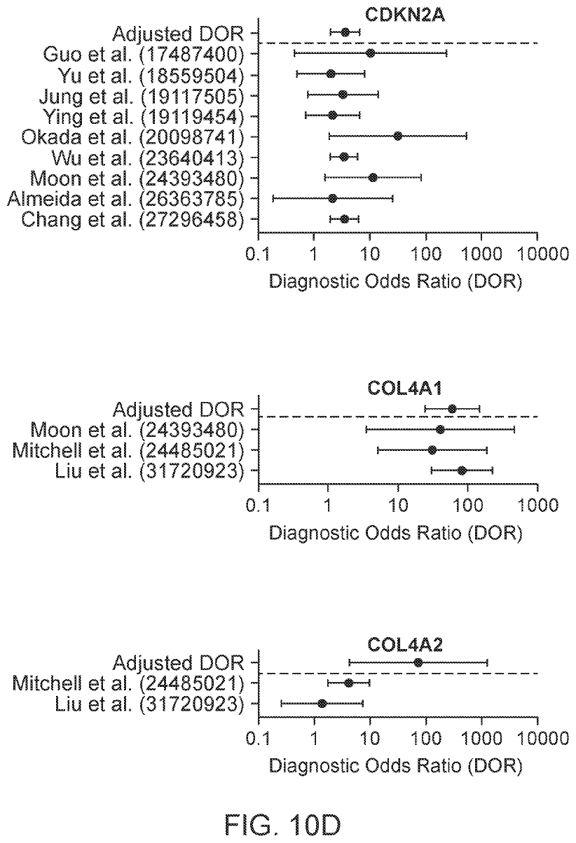

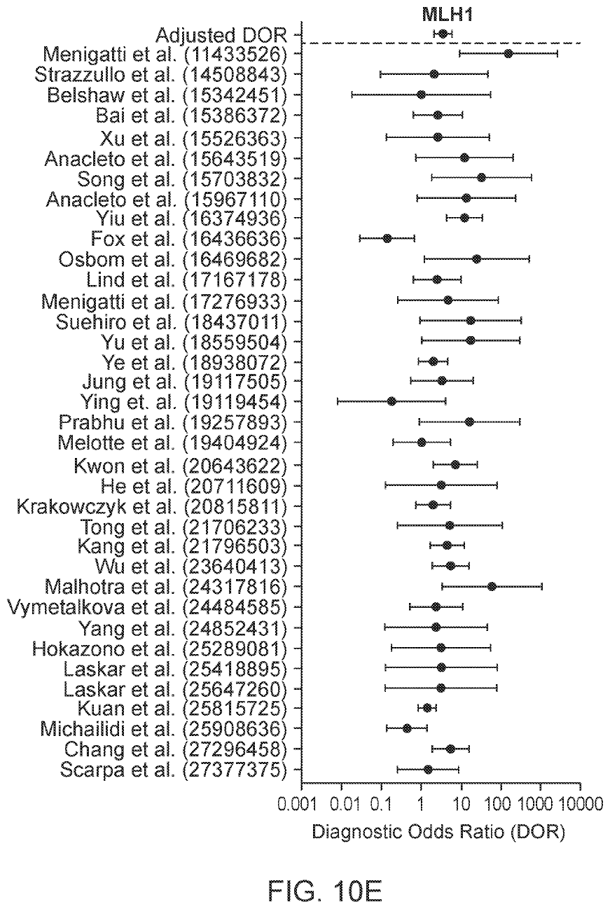

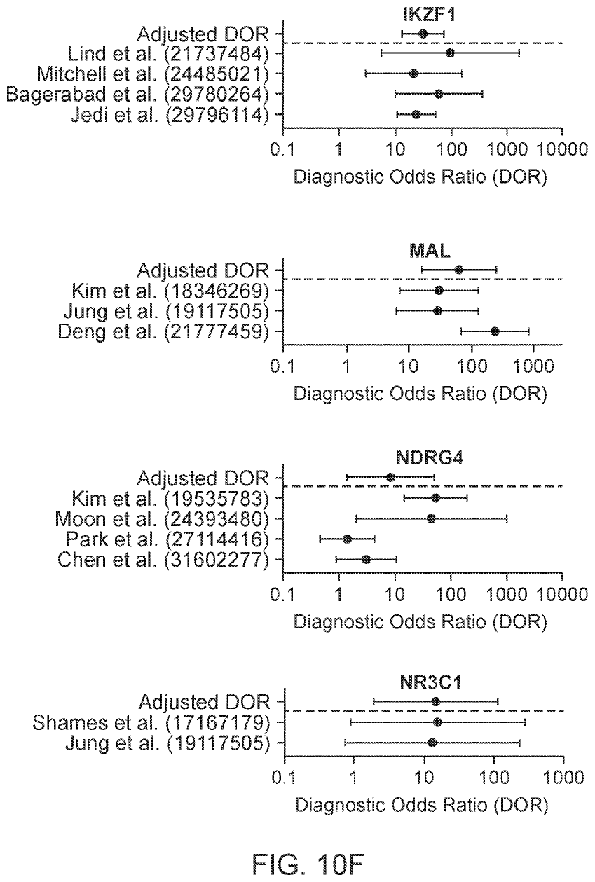

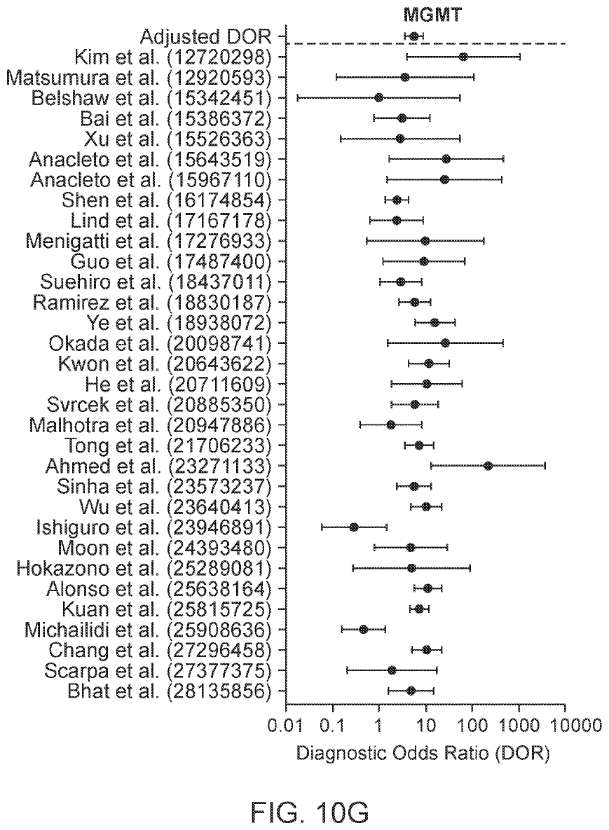

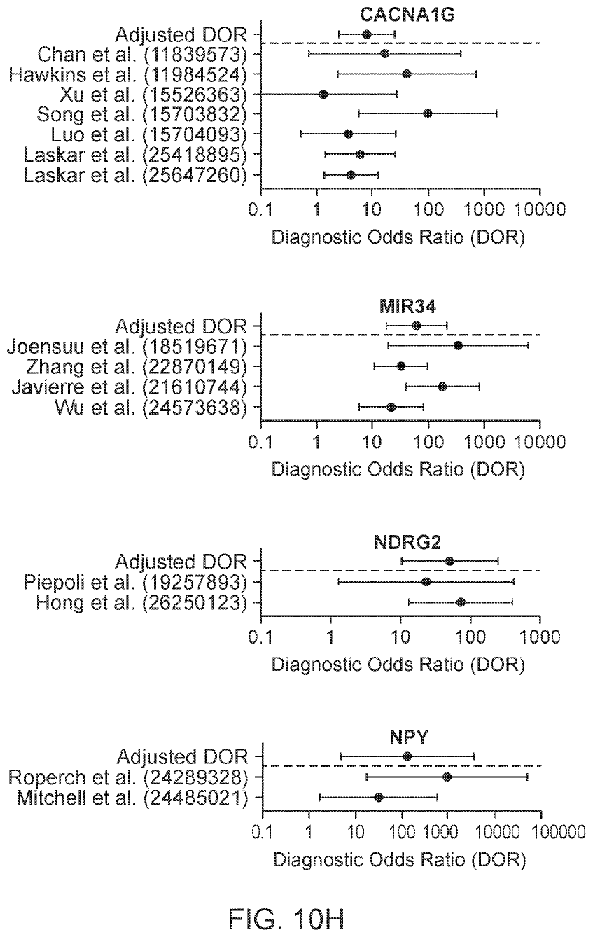

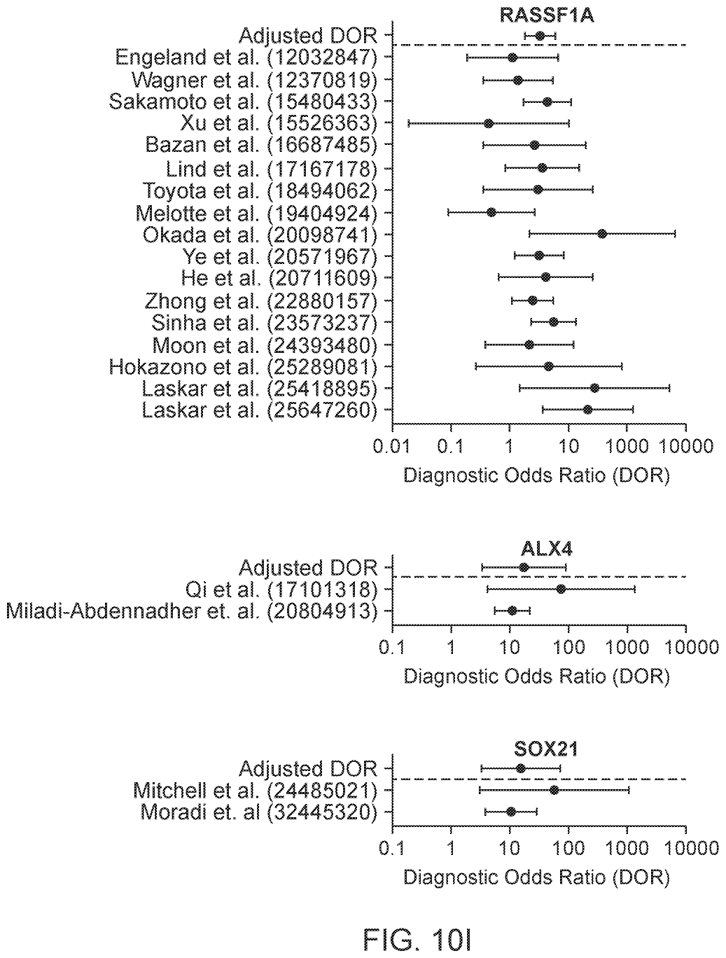

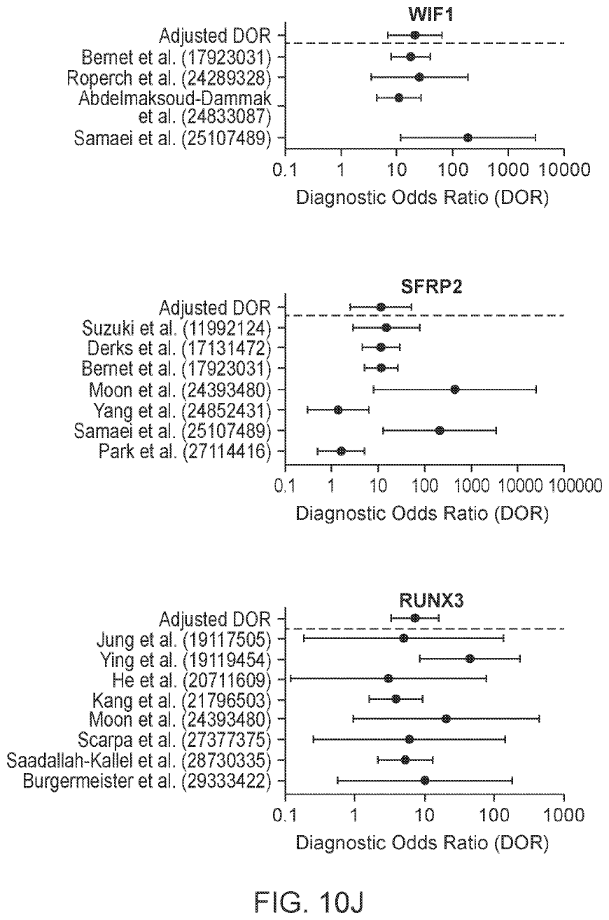

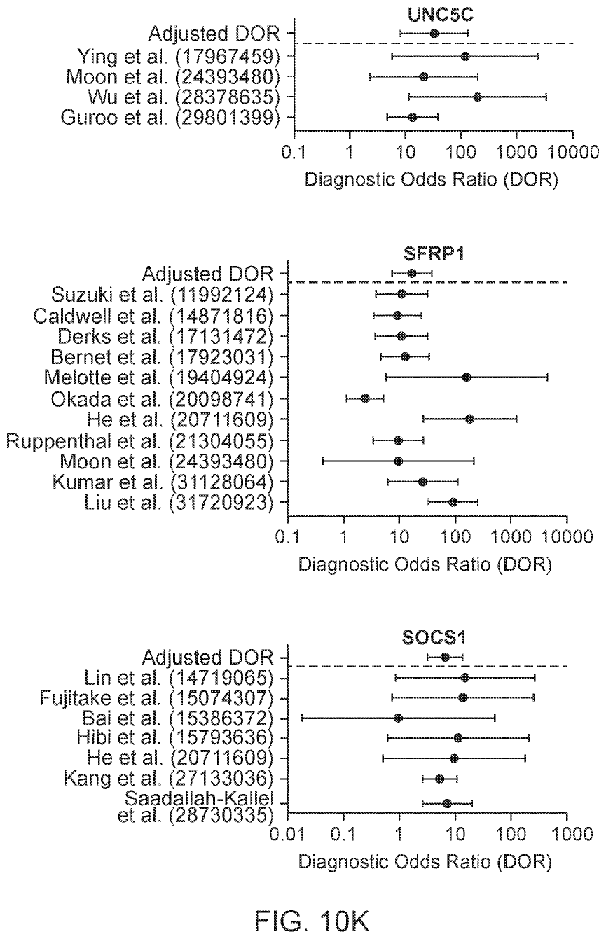

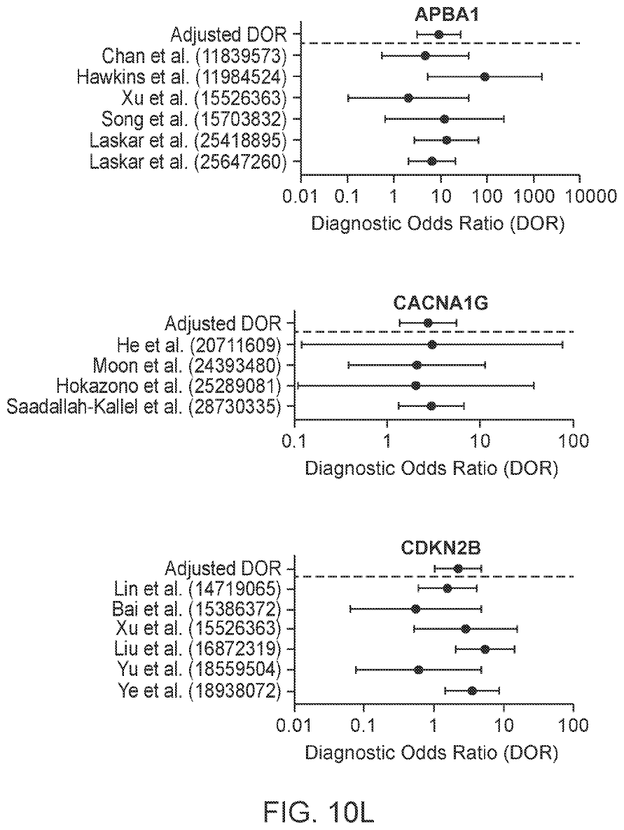

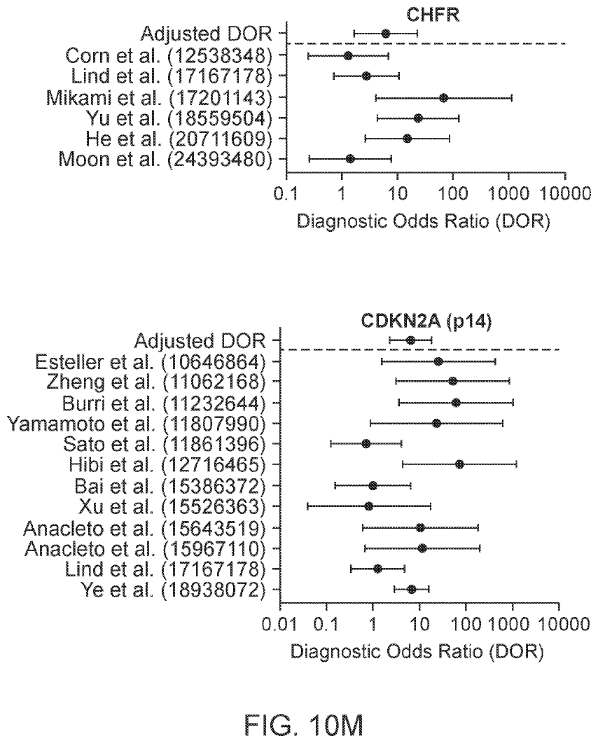

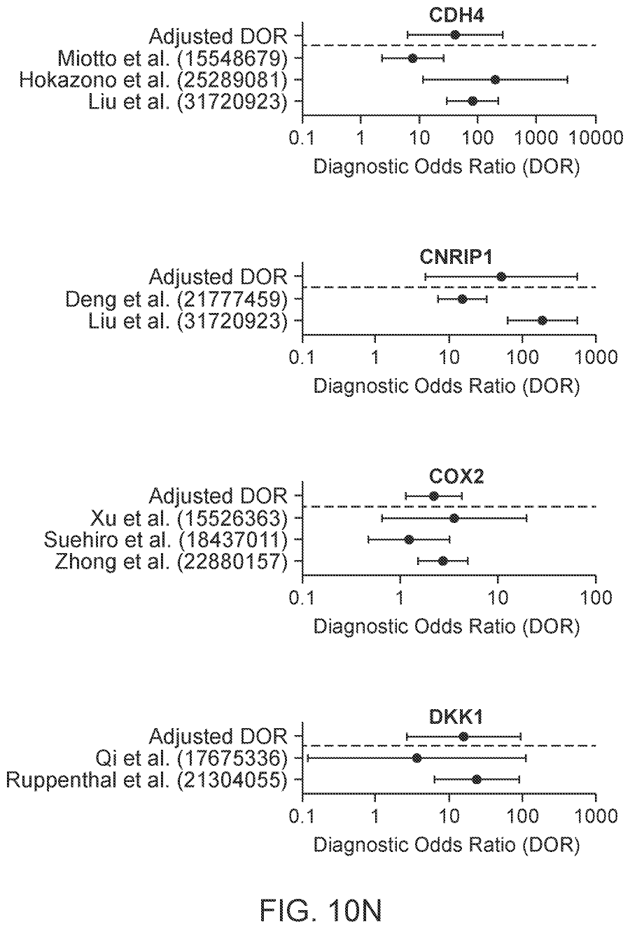

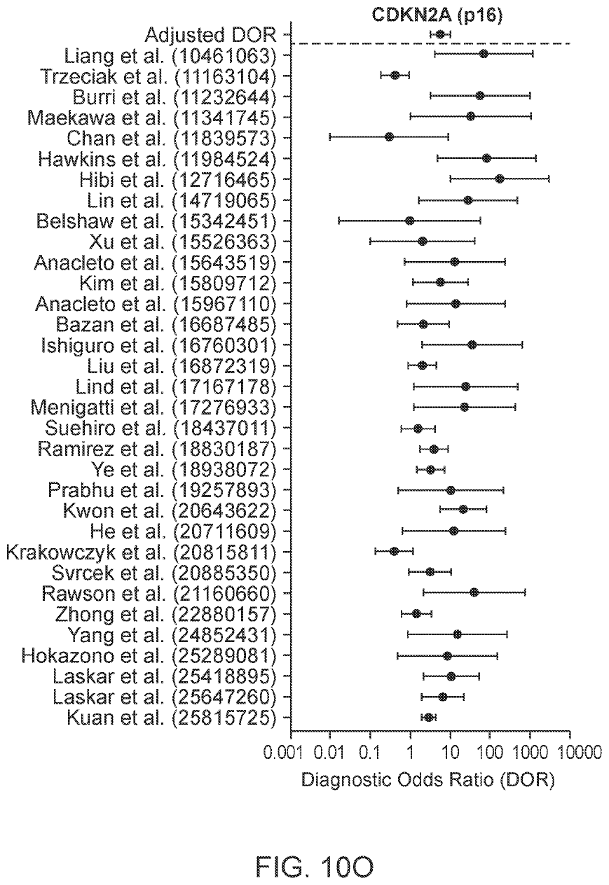

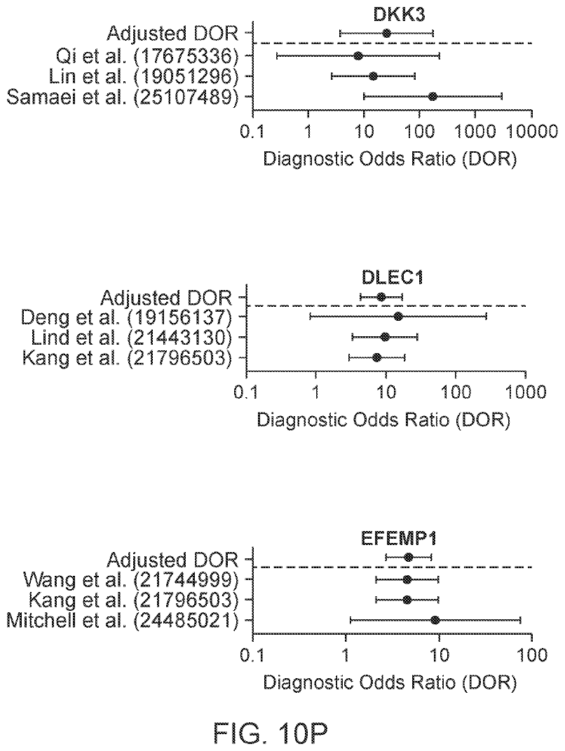

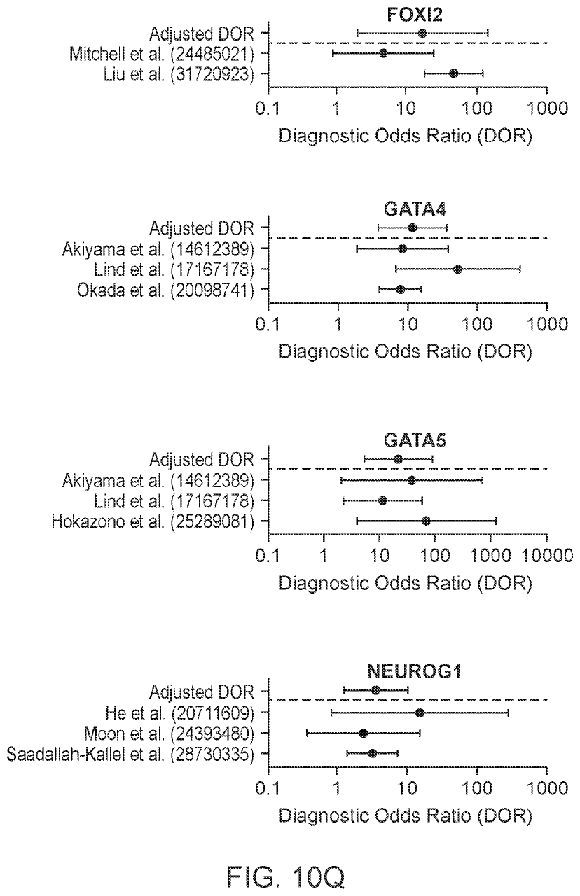

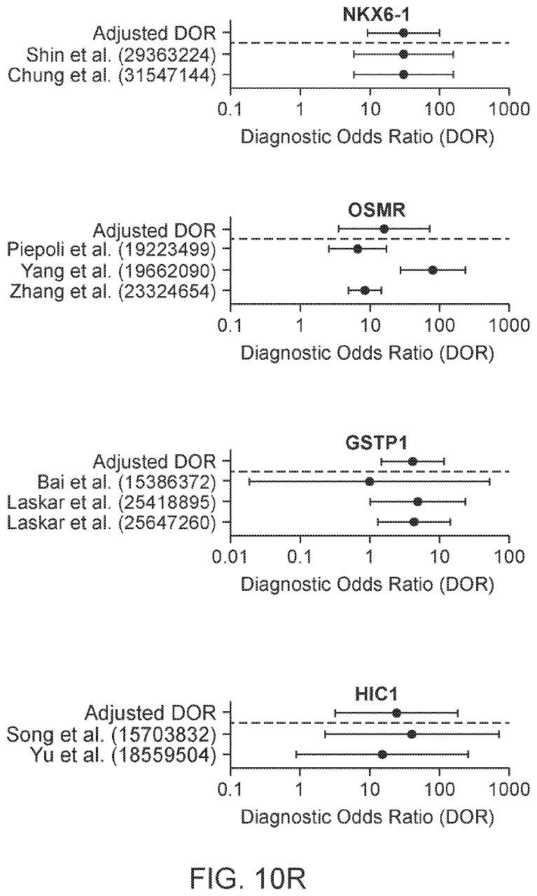

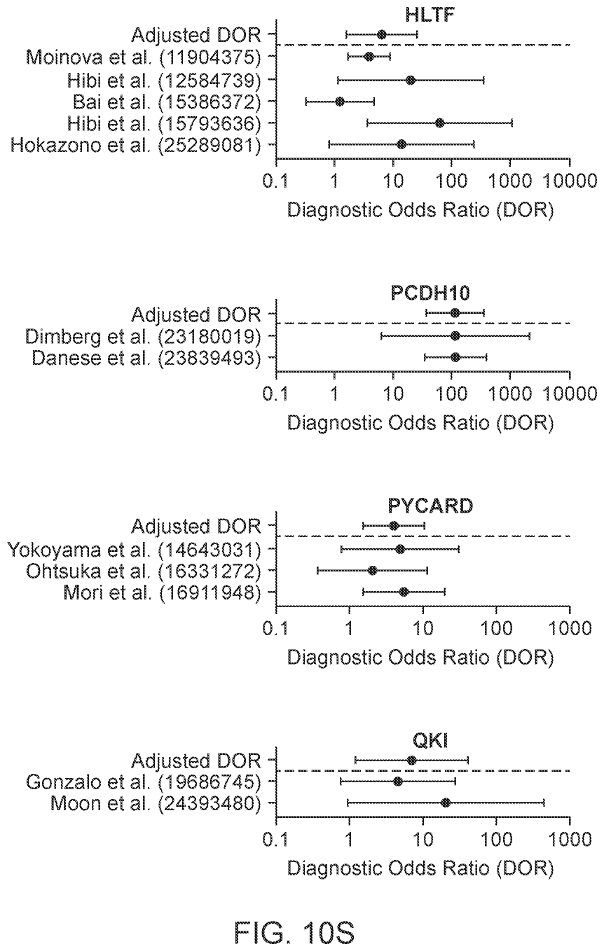

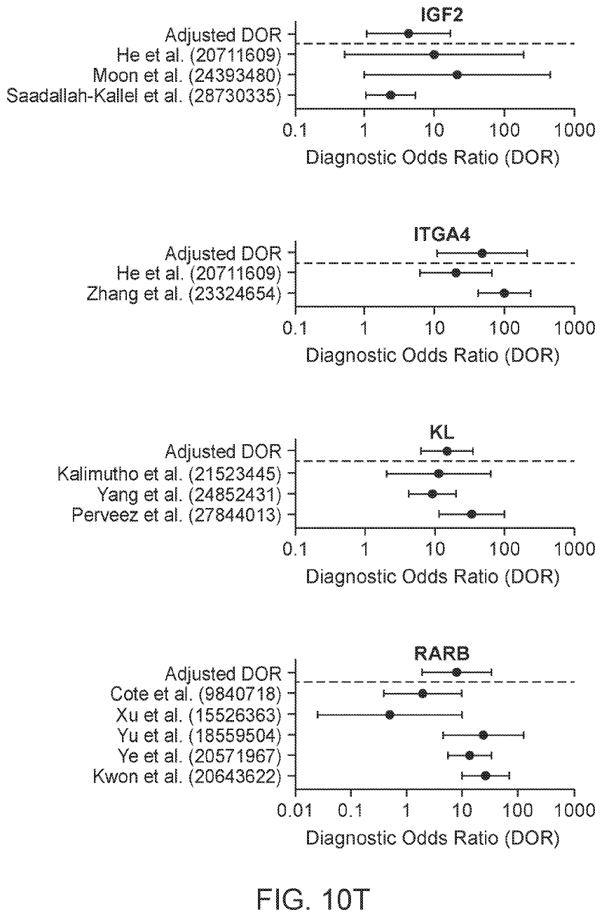

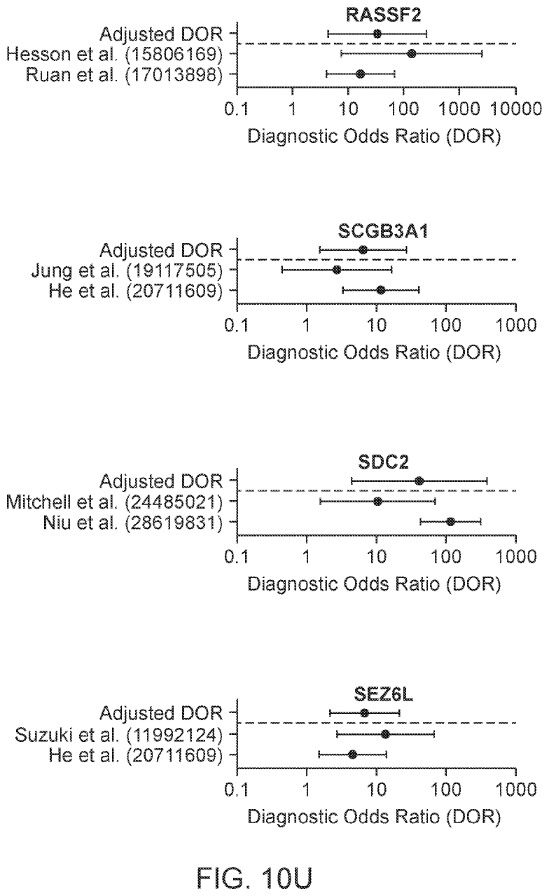

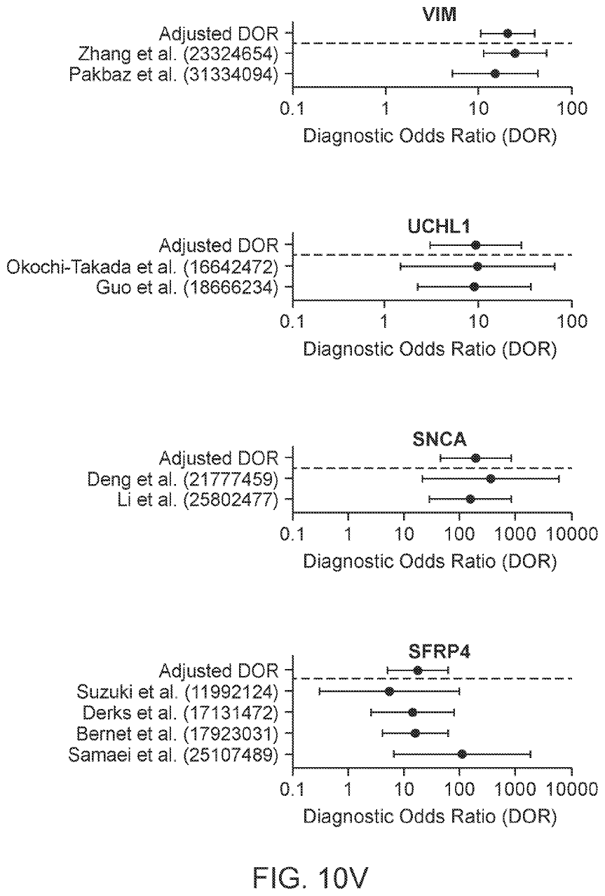

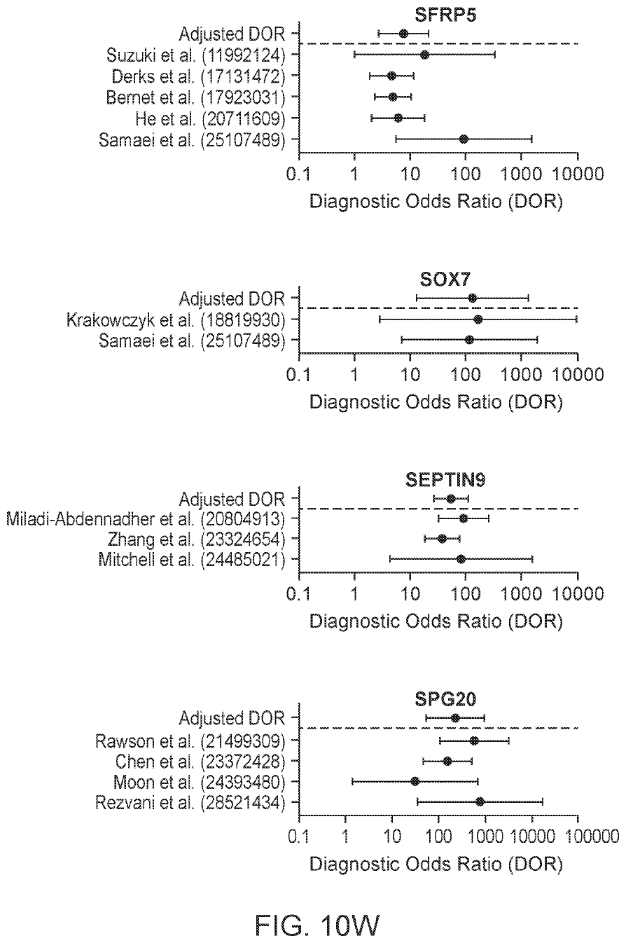

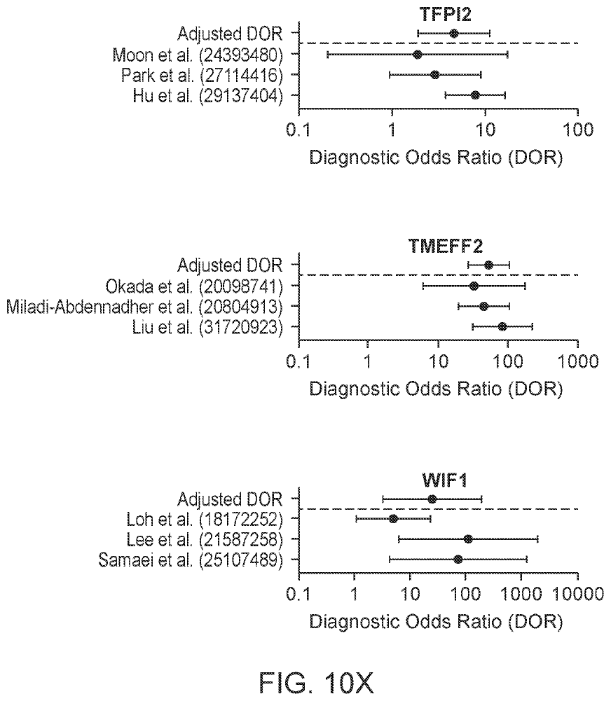

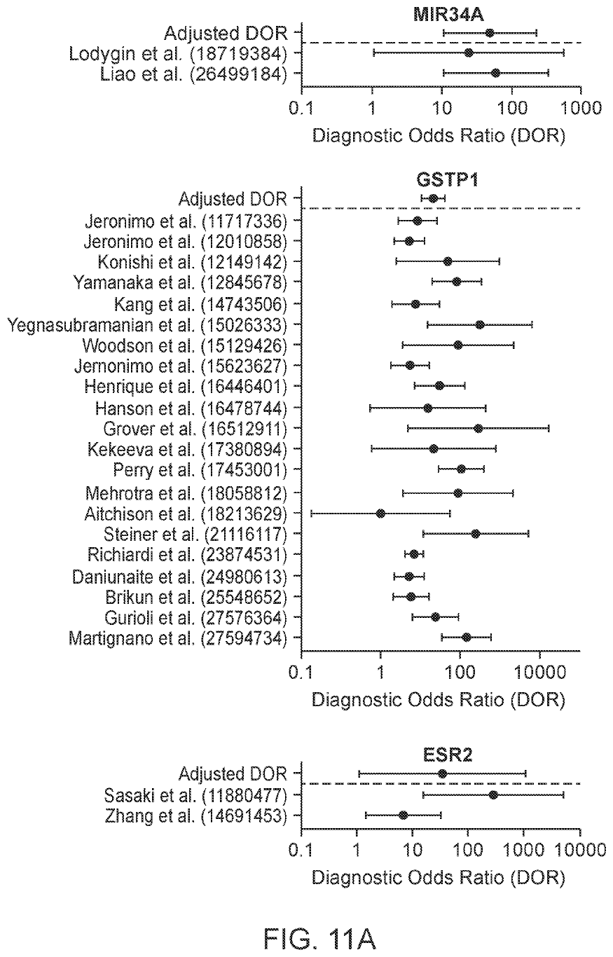

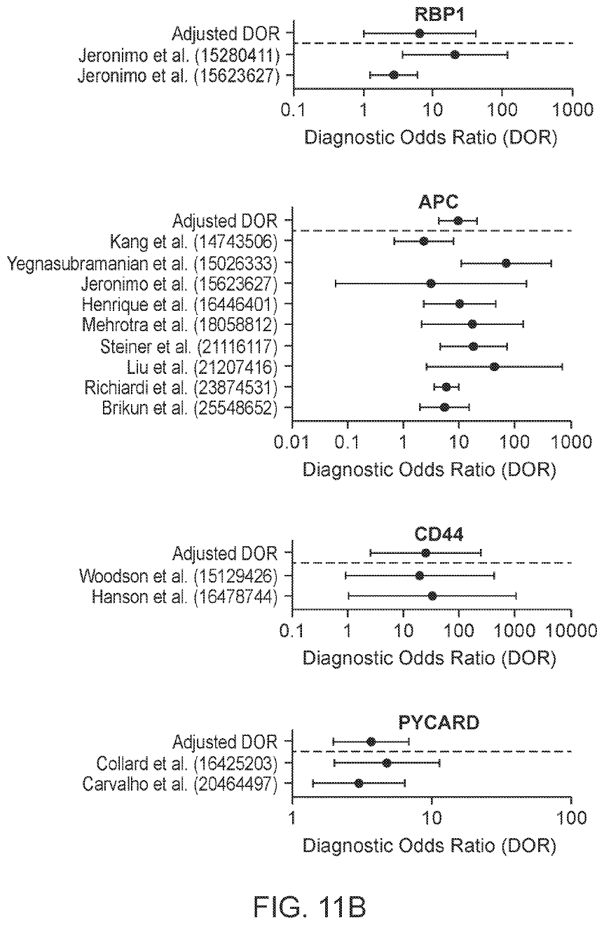

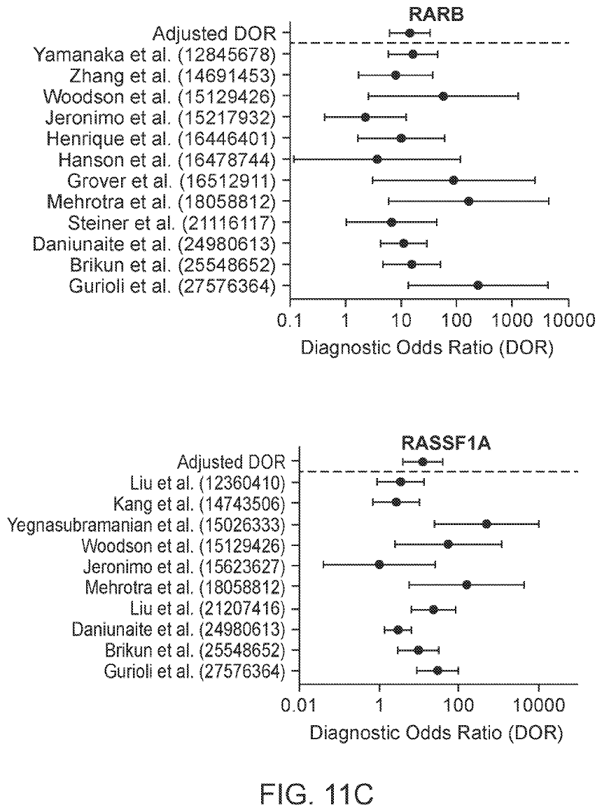

We utilized LAMB to discover methylated cfDNA biomarkers for HCC, CRC, PRAD, and non-small cell lung cancer (NSCLC). First, we used methylation data from 4,172 HCC patients to screen 33 genes and 249 CpGs through LAMB and discovered a methylated cfDNA HCC biomarker panel (LAMB-HCC) containing 21 CpGs in 10 genes (FIG. 1B and Table 1). With methylation data from 13,189 CRC patients, we then screened 92 genes and 596 CpGs with LAMB and discovered a CRC biomarker panel (LAMB-CRC) containing 46 CpGs in 17 genes (FIG. 1C and Table 2). Next, we utilized methylation data for 3,123 PRAD patients to screen 25 genes and 67 CpGs through LAMB and discovered a PRAD biomarker panel (LAMB-PRAD) containing 19 CpGs from five genes (FIG. 1D and Table 3). We then utilized methylation data from 7,412 NSCLC patients to screen 44 genes with LAMB to find a lung adenocarcinoma (LUAD) biomarker panel (LAMB-LUAD) containing 37 CpGs in 10 genes and a lung squamous cell carcinoma (LUSC) panel containing 30 CpGs in 9 genes (Supplementary Tables 10 and 11). Additional information on the datasets used for the LAMB biomarker discovery processes, meta-analysis results, and microarray results are provided in FIGS. 6 to 11, Supplementary Tables 1 to 9, and Supplementary Materials.

To determine if genes in the LAMB-HCC, LAMB-CRC, and LAMB-PRAD panel capture a wide range of tumor functions, we examined the genes in terms of their individual functions and their molecular pathways (Tables 1-3). Protein-protein interaction network (PPIN) analyses of the panels' genes showed that only the products of two LAMB-CRC genes (COL4A1, COL4A2) had protein-protein associations. Moreover, pathway enrichment analysis revealed that three LAMB-HCC genes (APC, WIF1, RUNX3) share the canonical Wnt signaling pathway, two LAMB-CRC genes (COL4A1, COL4A2) share the Type IV collagen protein pathway, and two LAMB-PRAD genes (RARB, RBP1) share the retinoic acid metabolism pathway (P<0.01).

### LAMB Biomarker Panels are Differentially Hypermethylated in Tumor Tissues

To identify the extent of hypermethylation of the LAMB-HCC, LAMB-CRC, and LAMB-PRAD panels in tumors, we evaluated LAMB panel methylation frequency scores (β_{LAMB}: β_{LAMB-HCC}, β_{LAMB-CRC}, β_{LAMB-PRAD}) in tumors with stage information (including 347 HCC, 365 CRC, and 502 PRAD), 50 liver ANTs, 45 colorectal ANTs, and 50 prostate ANTs from TCGA (26-28). β_{LAMB} panel scores were calculated by first taking the geometric mean of the methylation frequencies of the LAMB panel's CpGs (β_{CpG}) in a gene's promoter region to determine that gene's methylation frequency (β_{gene}). Then, β_{LAMB} was found through the geometric mean of these genes' methylation frequencies (see Methods). The geometric mean normalizes input frequencies so that all inputs are equally weighted in the output frequency. Accordingly, β_{gene} and β_{LAMB} equally weigh CpG and gene methylation frequencies, respectively, resulting in unbiased output methylation frequencies.

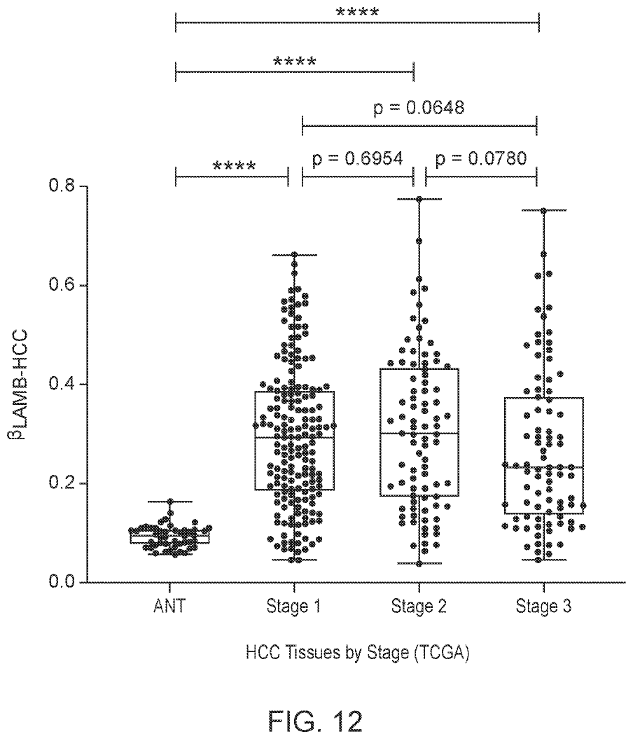

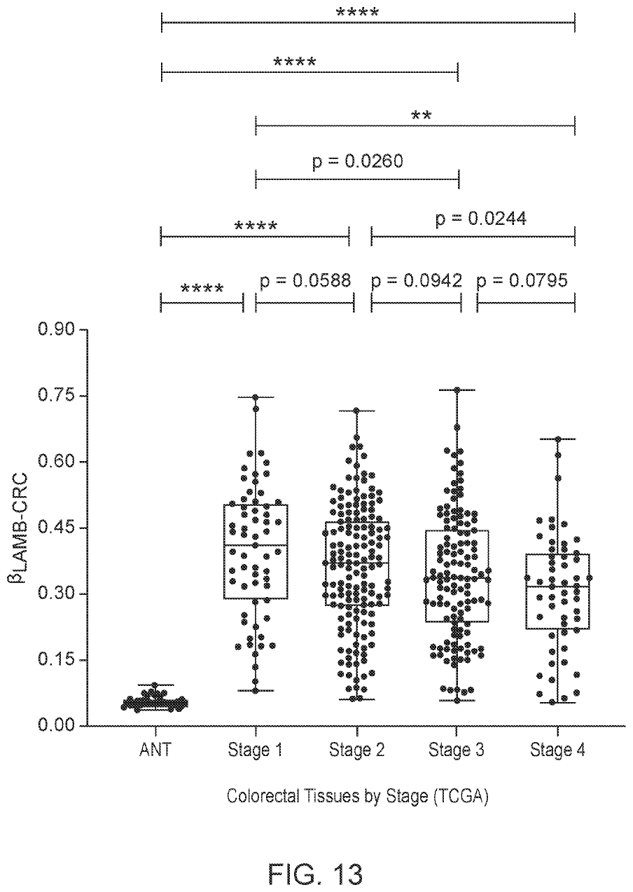

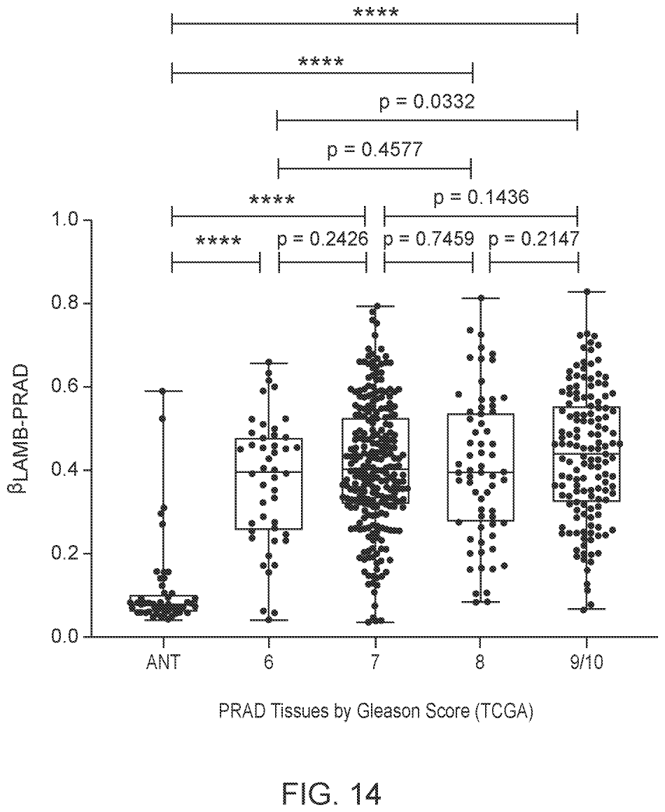

As expected, ANTs were significantly hypomethylated in terms of β_{LAMB} scores compared to cancers from respective tumor types in each stage (p<0.0001 for all stages; FIGS. 12 to 14). 71% of stage 1 HCCs were hypermethylated for the LAMB-HCC panel (β_{LAMB-HCC}>0.2) with an insignificant difference in β_{LAMB-HCC} scores between early and late-stage tumors (stage 1 vs. stage 2: p=0.6954; stage 1 vs. stage 3: p=0.0648; FIG. 12). Hypermethylation of LAMB-CRC (β_{LAMB-CRC}>0.2) was observed in 85% of stage 1 CRCs. Finally, 89% of PRADs with a Gleason Score of 6 were hypermethylated for LAMB-PRAD (β_{LAMB-PRAD}>0.2) with an insignificant difference in β_{LAMB-PRAD} between early and late-stage tumors (Gleason Score 6 vs. Gleason Score 7: p=0.2426; Gleason Score 6 vs. Gleason Score 8: p=0.4577; Gleason Score 6 vs. Gleason Score 9/10: p=0.0332; FIG. 14). The results suggest that LAMB panel hypermethylation typically occurs early in tumorigenesis and persists through later disease stages.

LAMB-HCC's Performance in cfDNA Highlights LAMB's Biomarker Screening Capabilities

We first tested LAMB's ability to filter out CpG biomarkers with low predictive power for diagnosing cancer patients in at-risk populations. A significant at-risk population for HCC is liver cirrhosis patients, who exhibit heavy liver scarring from chronic conditions such as viral hepatitis, alcoholic liver disease, and non-alcoholic steatohepatitis (29, 30). As a result, 90% of HCCs occur in liver cirrhosis patients. Surveillance of cirrhosis patients with high-sensitivity, high-specificity diagnostic tests is fundamental to detect HCCs for earlier treatment with improved outcomes (31). Standard-of-care HCC surveillance combines serum alpha-fetoprotein (AFP) tests with ultrasound imaging because the testing accuracy of AFP alone is ˜70% (32, 33). However, this monitoring is both costly and cumbersome, leading to poor adherence and ineffective surveillance (34).

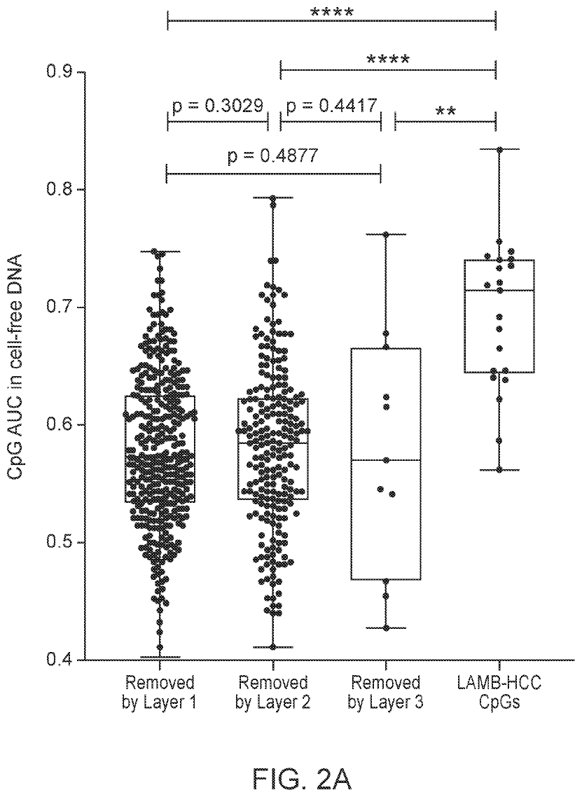

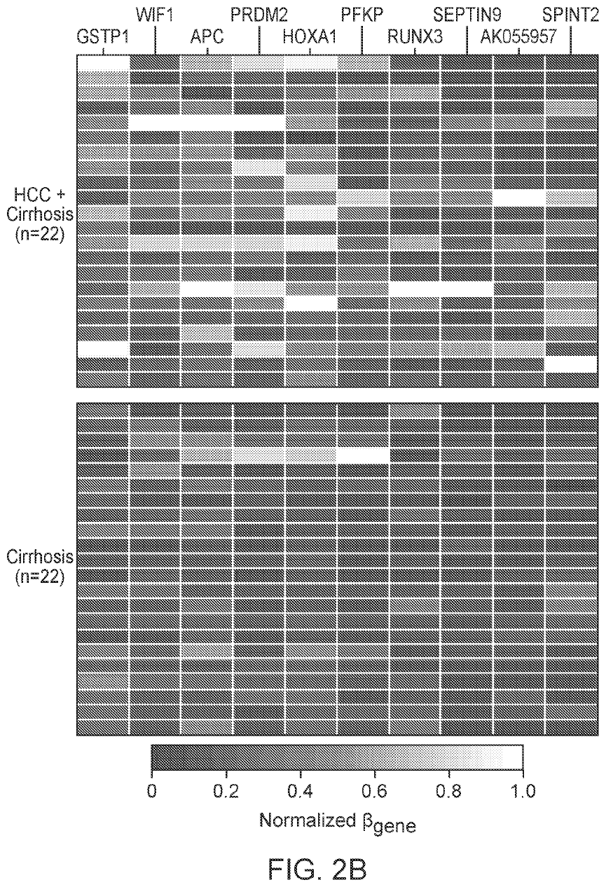

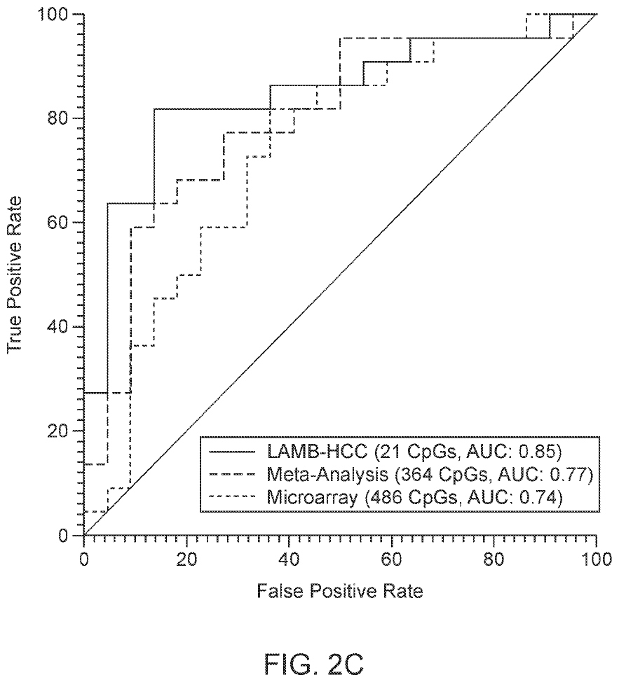

To assess LAMB's ability to select predictive biomarkers, we evaluated the CpGs screened by LAMB on an independent 450K validation dataset of cfDNA from 22 HCC patients with liver cirrhosis and 22 non-HCC patients with liver cirrhosis who were matched by liver function (35). AUCs for distinguishing HCC patients from non-HCC patients were found for each CpG screened through LAMB. These AUCs showed the utility of LAMB's multi-layered screening, as LAMB-HCC CpGs showed increased predictive power when compared to CpGs removed by LAMB Layer 1, Layer 2, and Layer 3 (p<0.0001, p<0.0001, and p<0.01, respectively; FIG. 2A).

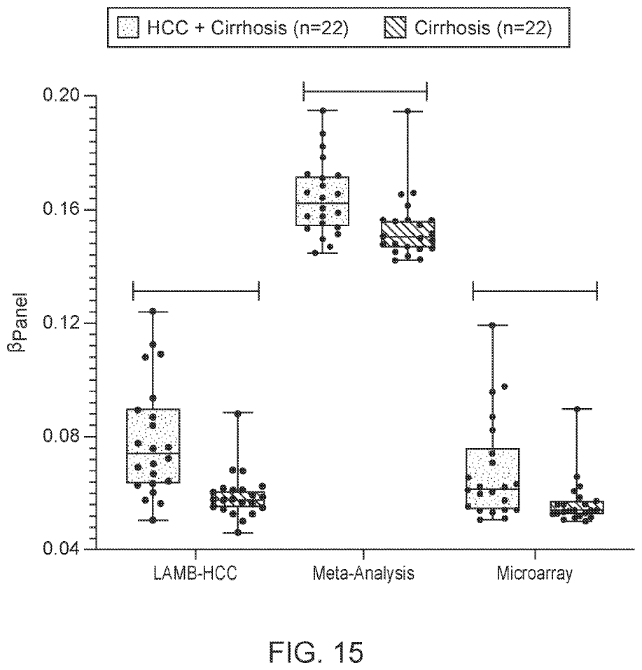

To evaluate LAMB-HCC biomarkers on the validation data, we calculated β_{gene} and β_{LAMB-HCC} scores for each patient. β_{gene} scores demonstrated moderate predictive ability for differentiating HCC patients and non-HCC patients [AUC, range of 0.64 to 0.77] (FIG. 2B and Table 1). However, β_{LAMB-HCC} scores demonstrated high predictive power [AUC, 0.85; 95% confidence intervals (CI), 0.73 to 0.97] (p<0.0001; FIG. 2C and FIG. 15), suggesting that combining β_{gene} scores into a β_{LAMB-HCC} score produced a more tumor-specific methylation signature in the patients.

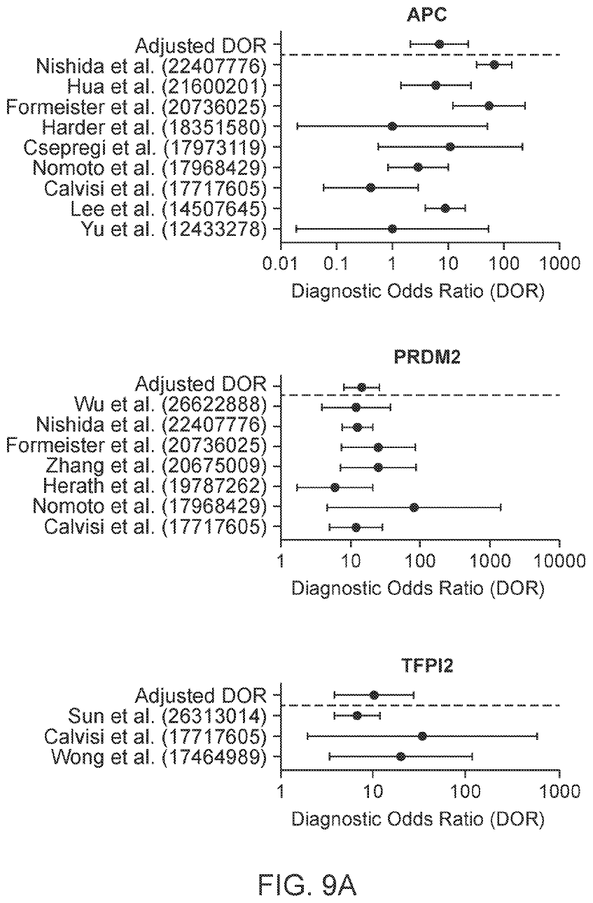

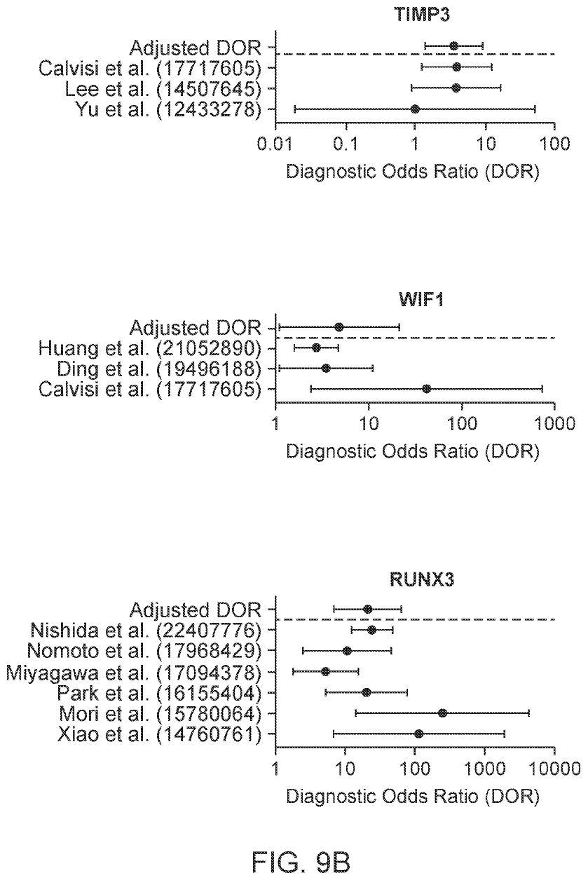

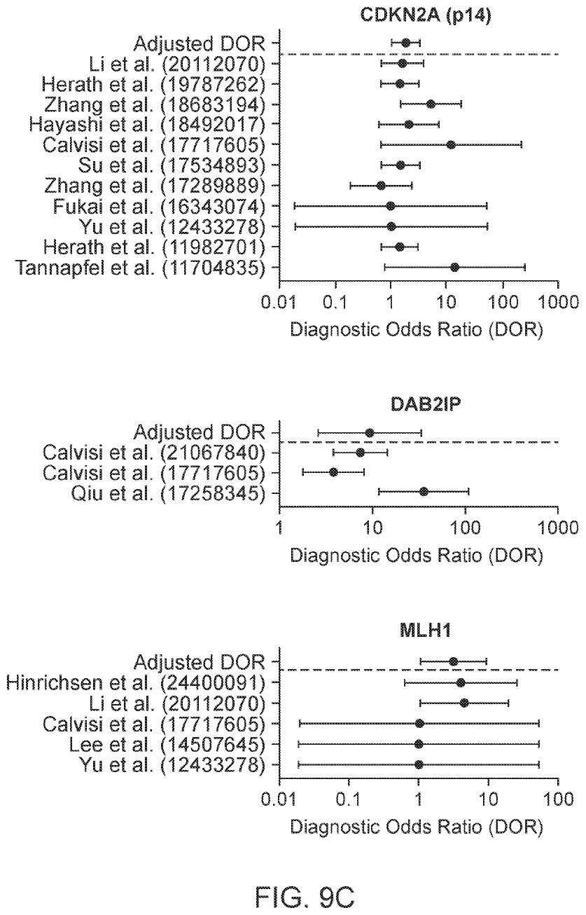

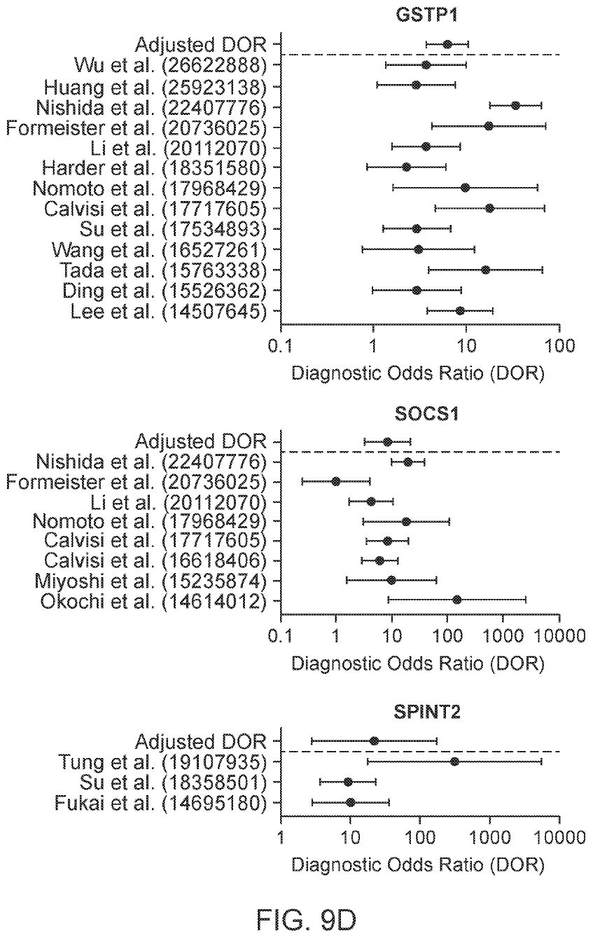

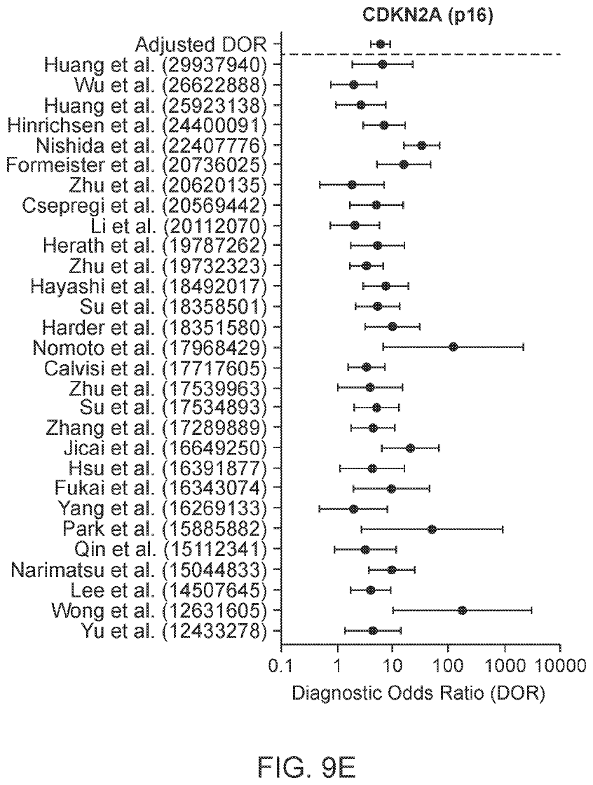

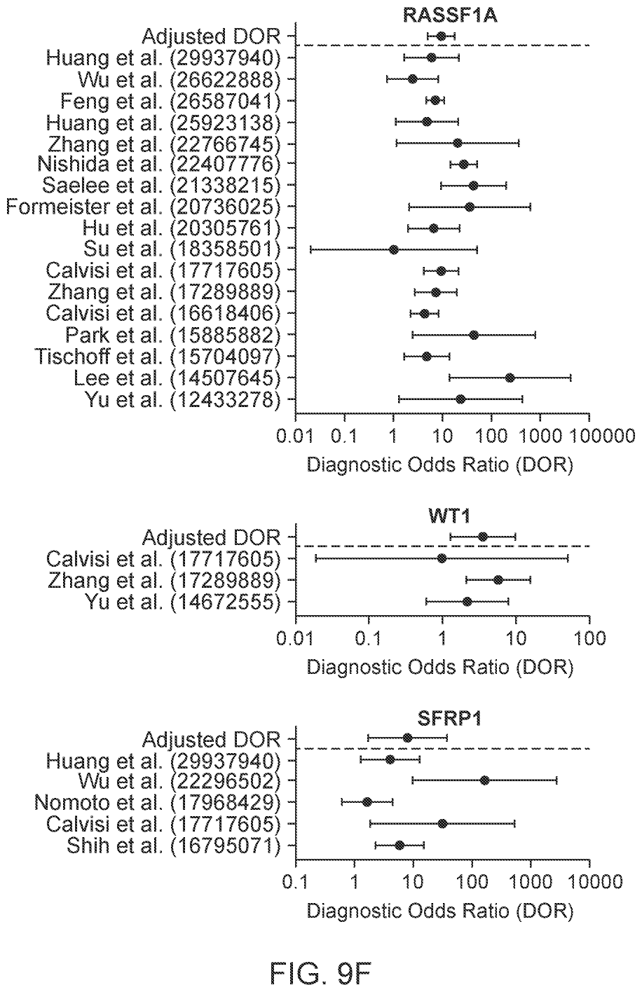

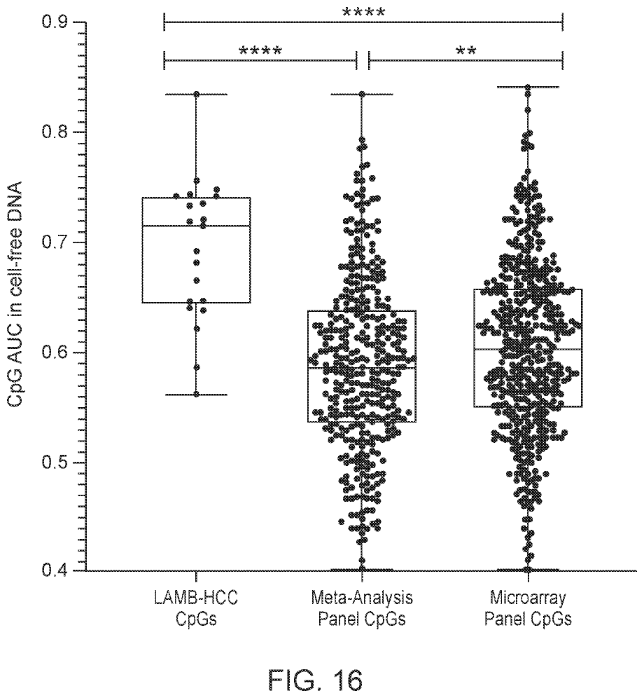

LAMB's combination of meta-analysis and microarray data led to a more predictive set of biomarkers than using either approach alone. LAMB-HCC outperformed a “meta-analysis” panel comprised of all 364 promoter CpGs in the 450K microarray for the 22 genes identified by LAMB Layer 1 [AUC, 0.77; 95% CI, 0.63 to 0.91] (p<0.01; FIGS. 2C and 9), as well as a “microarray” biomarker panel made up of 486 promoter CpGs in 267 genes found by applying LAMB Layers 2 and 3 to all of the promoter CpGs in the microarray [AUC, 0.74; 95% CI, 0.59 to 0.89] (p<0.01; FIGS. 2C and 9). LAMB-HCC CpGs also had higher median predictive power than CpGs in meta-analysis and microarray panels (p<0.0001 and p<0.0001, respectively; FIG. 16).

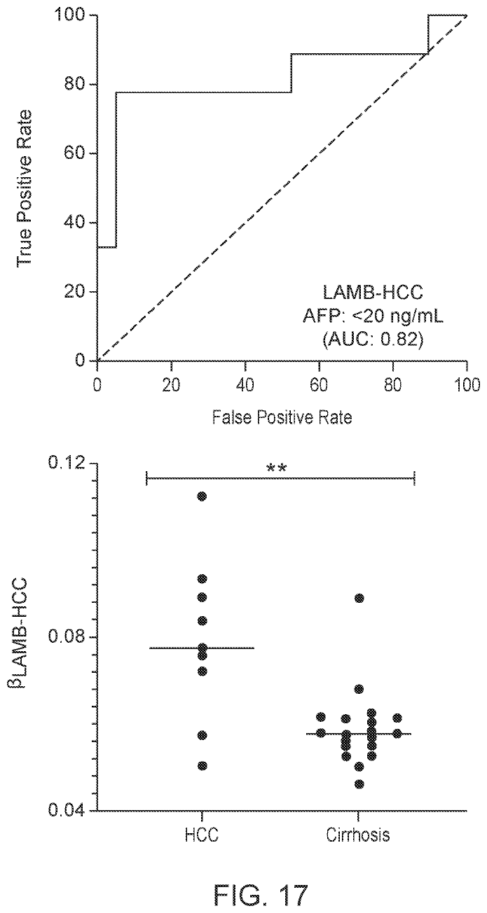

We next evaluated how LAMB-HCC performs at detecting HCC in patients misdiagnosed by AFP in this dataset. Three cirrhosis patients without AFP values were excluded. At the clinical cutoff of 20 ng/mL for AFP, 13 of 22 HCC patients tested positive, a 59% sensitivity that is in line with large studies (30, 33, 36). β_{LAMB-HCC} scores showed high predictive power for differentiating the nine remaining misdiagnosed HCC patients and 19 cirrhosis patients [AUC, 0.82; 95% CI, 0.61 to 1.00], suggesting that LAMB-HCC can be used to complement AFP testing (p<0.01; FIG. 17).

LAMB-CRC Detects Early-Stage Colorectal Tumors Through cfDNA Samples

We next assessed how a LAMB biomarker panel performs for detecting tumors in average-risk populations. Many health guidelines suggest stool-based or visual-based CRC surveillance for average-risk individuals above the age of 45 or 50. Blood-based CRC tests can improve adherence, resulting in effective surveillance (37-38). To evaluate LAMB-CRC's diagnostic capability, we utilized an independent targeted bisulfite sequencing validation dataset of cfDNA from 38 CRC patients and 46 healthy controls matched by age (39). Methylated and unmethylated counts of the LAMB-CRC CpG biomarkers and adjacent CpGs were extracted to create high-resolution β_{gene} scores (see Methods). LAMB-CRC β_{gene} scores demonstrated moderate to high predictive ability for differentiating CRC patients from healthy controls [AUC, range of 0.56 to 0.85] (Table 2).

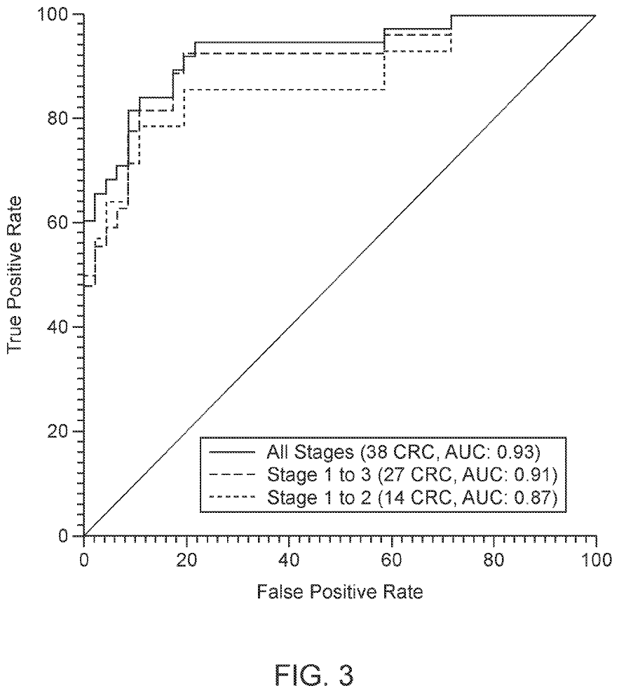

By comparison, β_{LAMB-CRC} scores showed very high predictive power [AUC, 0.93; 95% CI, 0.87 to 0.99] (p<0.0001; FIG. 3), further suggesting that combining β_{gene} scores into a β_{LAMB} score produced a more tumor-specific methylation signature. LAMB-CRC's predictive power was not restricted to metastatic tumors. When evaluating 27 CRC patients with stage 1 to 3 tumors, β_{LAMB-CRC} scores continued to show very high predictive power [AUC, 0.91; 95% CI, 0.84 to 0.98] (p<0.0001; FIG. 3). Similarly, β_{LAMB-CRC} scores showed high predictive power [AUC, 0.87; 95% CI, 0.75 to 1.00] (p<0.0001; FIG. 3) when testing 14 CRC patients with localized, early-stage tumors.

LAMB-PRAD Signature in cfDNA Reflects Therapy Response and Tumor Burden

We also examined if a LAMB biomarker panel could reflect tumor burden and be used to monitor tumor response to therapy. We leveraged LAMB-PRAD to evaluate an existing dataset of longitudinal cfDNA samples for PRAD patients undergoing treatment. PRAD patients were an ideal population for this proof-of-concept study, as serum-based prostate-specific antigen (PSA) is recommended by multiple urological organizations to monitor PRAD patients and classify their tumor response to therapy (40-44). Defining therapy responders and therapy non-responders along clinically established criteria of PSA change, we hypothesized that responders will show a greater decrease in LAMB biomarker signal after starting treatment than non-responders.

The LAMB-PRAD panel was tested on an independent 450K validation dataset of cfDNA from multiple visits for 23 metastatic, castration-resistant prostate cancer (CRPC) patients treated with abiraterone acetate (AA) and docetaxel (45). For all patients, methylation data for a baseline pre-treatment sample and at least one on-treatment sample was available. Past studies have shown that CRPC patients treated with AA who exhibit a 30% decrease in PSA at their first on-treatment visit demonstrate significant improvements in outcome (46-49). In this cohort, nine patients were classified as responders by this criterion, and the 14 remaining patients were non-responders.

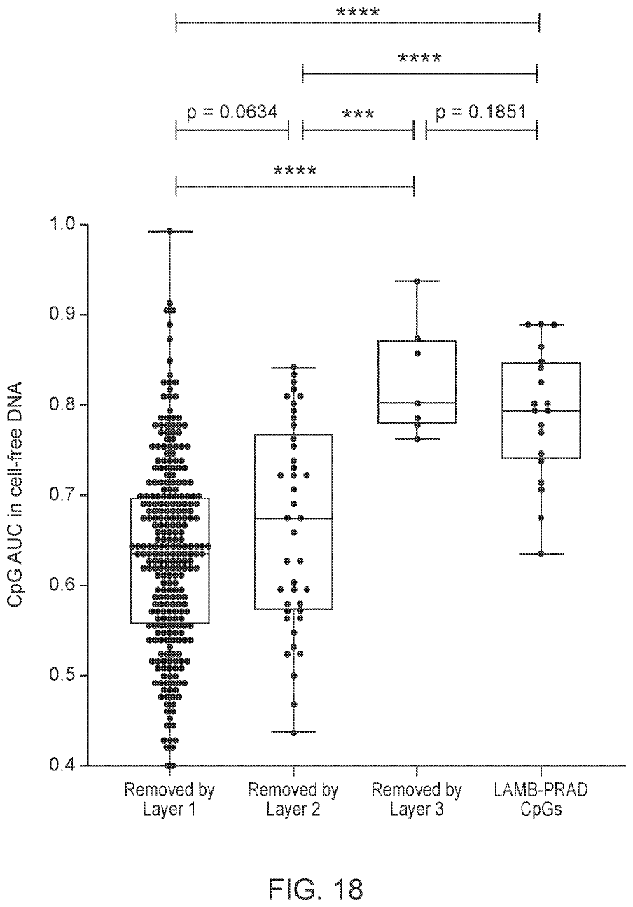

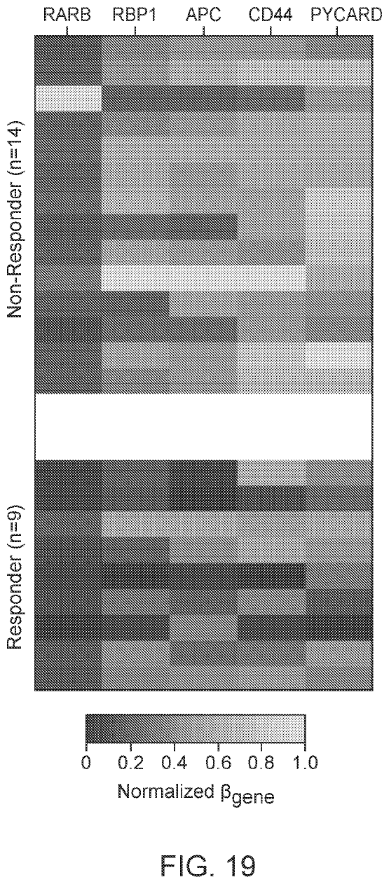

We calculated β_{gene} and β_{LAMB-PRAD} scores for all visits. To test LAMB-PRAD's ability to distinguish therapy responders from non-responders, percent difference in β_{CpG}, β_{gene}, and β_{LAMB-PRAD} scores for the first on-treatment visit compared to the baseline visit was found for all patients. Individual LAMB-PRAD CpGs showed higher median predictive power than CpGs removed by LAMB Layers 1 and 2 (p<0.01, FIG. 18). β_{gene} scores exhibited high predictive ability [AUC, range of 0.79 to 0.89] (Table 3, FIG. 19), and β_{LAMB-PRAD} scores had very high predictive power [AUC, 0.90; 95% CI, 0.75 to 1.00] (p<0.001; FIGS. 3A and 3B).

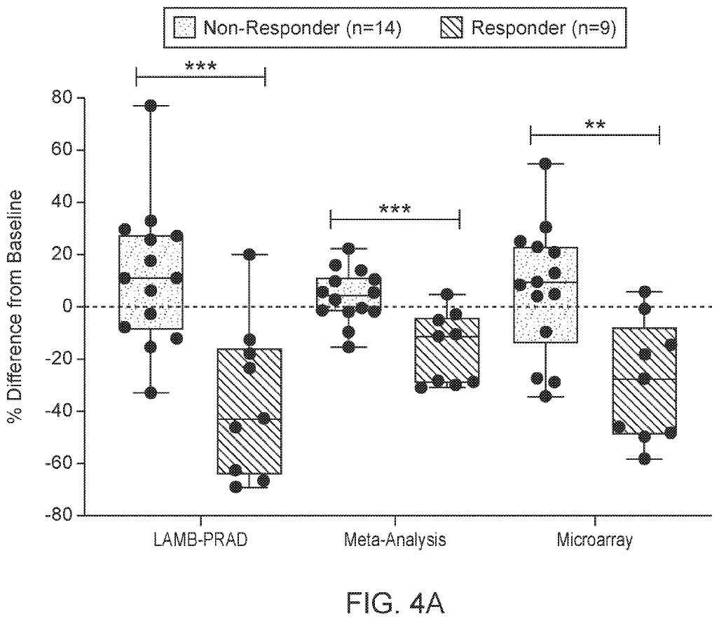

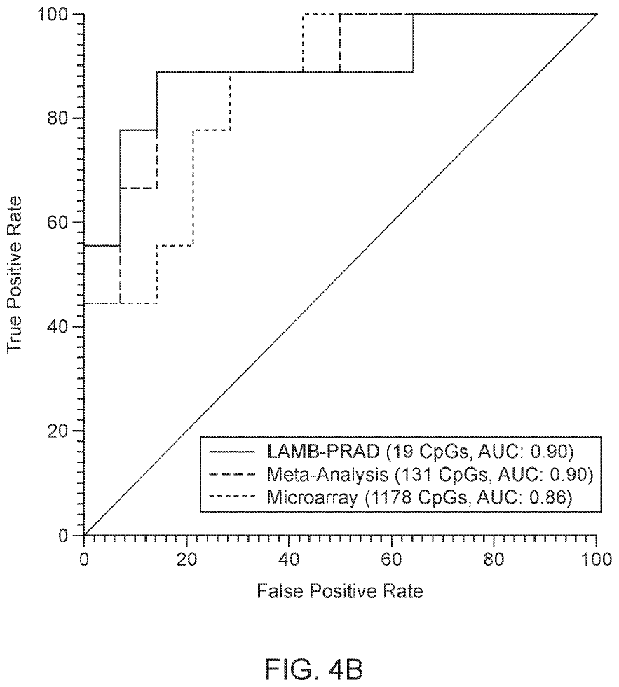

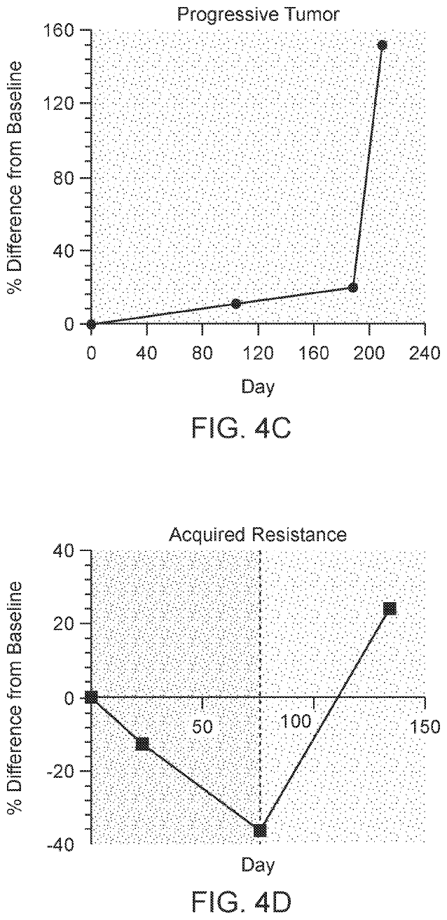

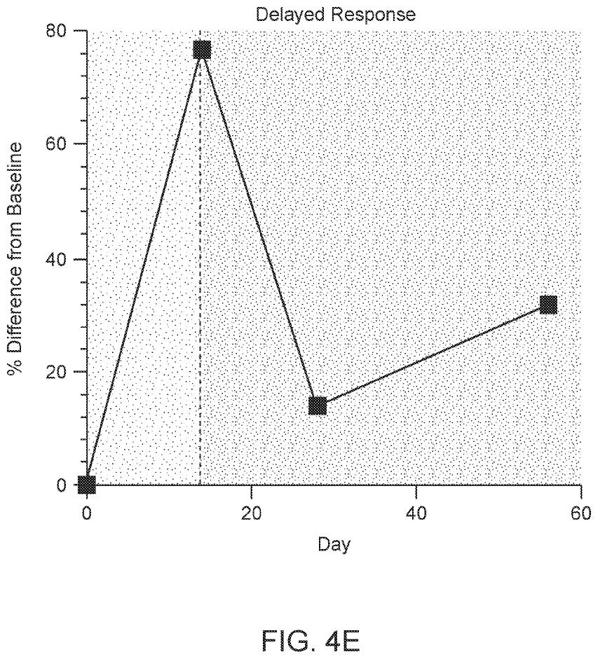

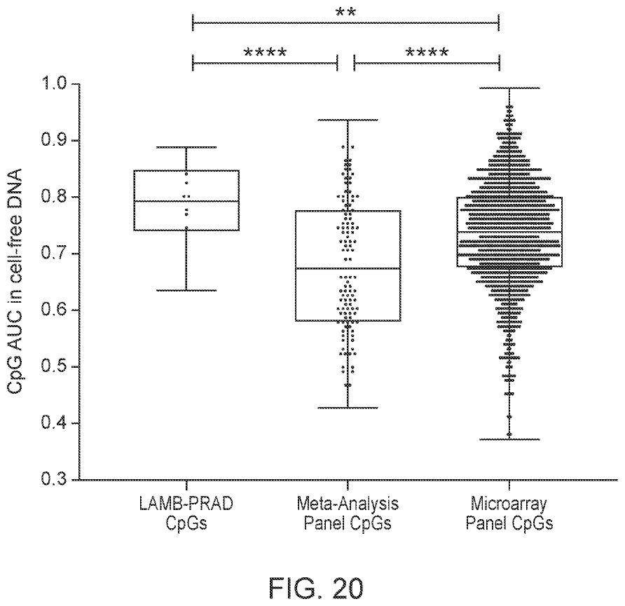

LAMB-PRAD outperformed a microarray panel comprised of 1178 promoter CpGs in 624 genes from applying LAMB Layers 2 and 3 to all promoter CpGs in the 450K microarray [AUC, 0.86; 95% CI, 0.71 to 1.00] (p<0.01; FIGS. 4A and 4B); however, a meta-analysis panel made up of 131 promoter CpGs in the nine genes from LAMB Layer 1 showed similar predictive power to LAMB-PRAD [AUC, 0.90; 95% CI, 0.76 to 1.00] (p<0.001; FIGS. 4A and 4B). Nevertheless, the separation in median percent difference of panel scores between responders and non-responders is significantly greater for the LAMB-PRAD panel than the meta-analysis panel [53.79% v. 15.35%, respectively] (FIG. 4A). LAMB-PRAD CpGs showed higher median predictive power than CpGs in the meta-analysis and microarray panels (p<0.001 and p<0.01, respectively; FIG. 20).

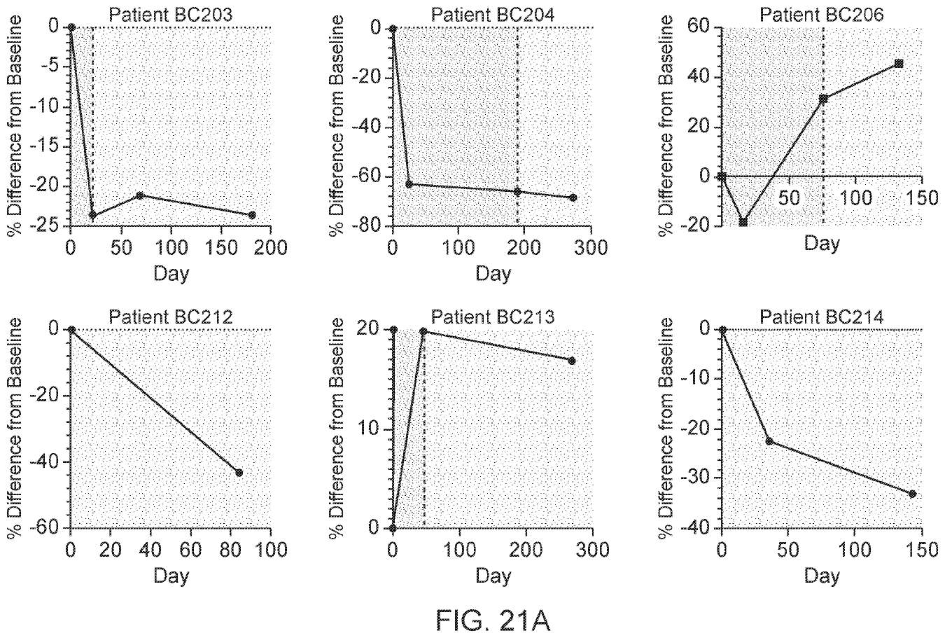

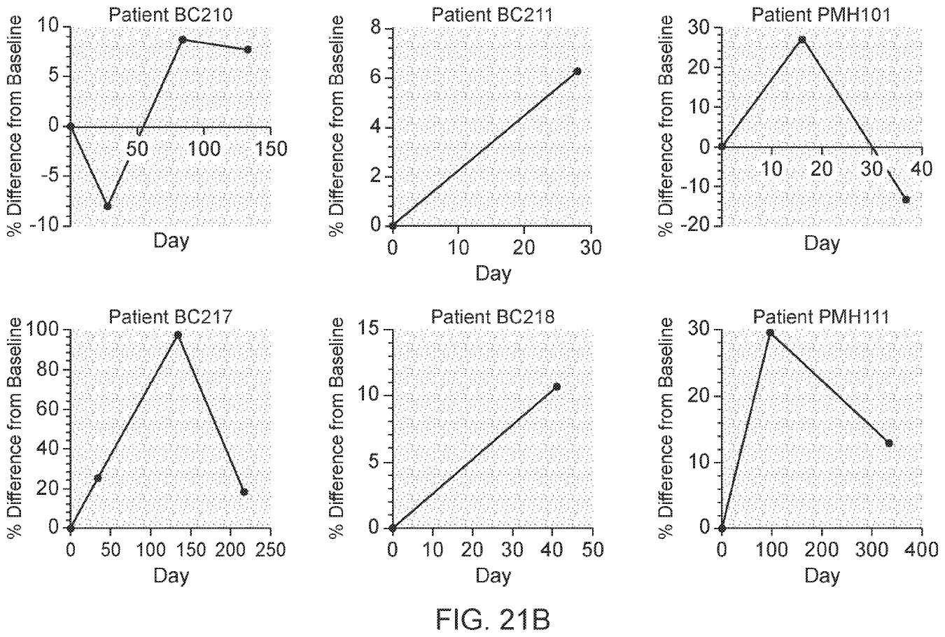

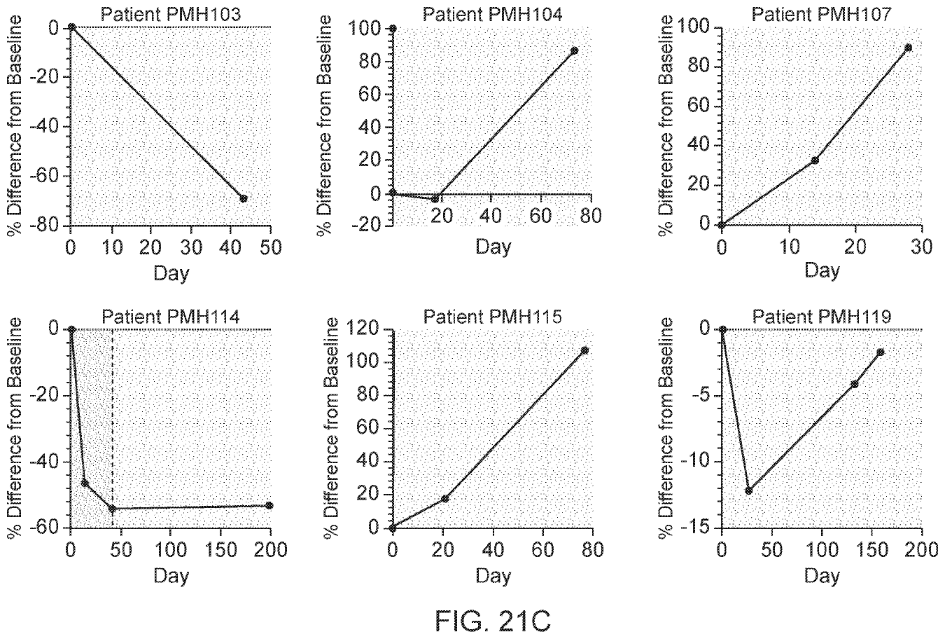

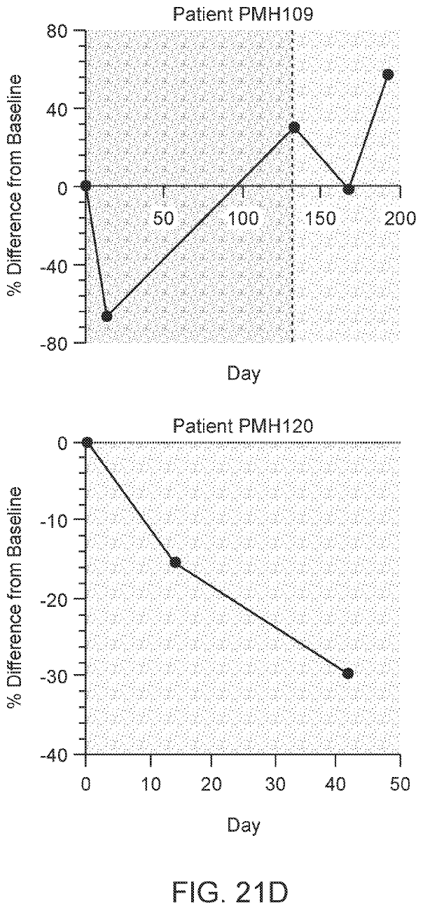

We then examined LAMB-PRAD's ability for longitudinally monitoring therapy response. Beyond the first on-treatment visit, the dataset contained 24 additional on-treatment visits in which the patient's PSA indicated that their tumor was progressing and seven visits in which the patient's PSA indicated that the tumor was responding. In 19 of the 24 progressive visits, β_{LAMB-PRAD} scores were greater than the patient's nadir β_{LAMB-PRAD} score, while in five of the seven responding visits, β_{LAMB-PRAD} scores were lower than the patient's peak β_{LAMB-PRAD} score (FIGS. 4C to 4E, and FIG. 21). These results suggest that β_{LAMB-PRAD} scores correlate with PRAD tumor progression and response after the first on-treatment visit.

LAMB-LUAD and LAMB-LUSC for Detection of Early-Stage NSCLC Through cfDNA Samples

A lung adenocarcinoma (LUAD) biomarker panel (LAMB-LUAD) containing 37 CpGs in 10 genes and a lung squamous cell carcinoma (LUSC) panel containing 30 CpGs in 9 genes are listed in Table 13 (Methylation frequency and AUC values for LAMB-LUAD CpGs) and Table 14 (Methylation frequency and AUC values for LAMB-LUSC CpGs), respectively. However, at the writing of this disclosure, we have not found suitable validation datasets of cfDNA samples from NSCLC patients to independently evaluate the predictive accuracy of these two panels as we did for LAMB-HCC, LAMB-CRC, and LAMB-PRAD. Nevertheless, the rationale and biological significance of LAMB-LUAD and LAMB-LUSC hold their own, thus we anticipate they will perform at satisfactory levels in an independent validation dataset (when available), similar to what have already been demonstrated for the other three cancers.

LAMB Panels are Tumor-Specific with Hypomethylation in Other Cancer Types

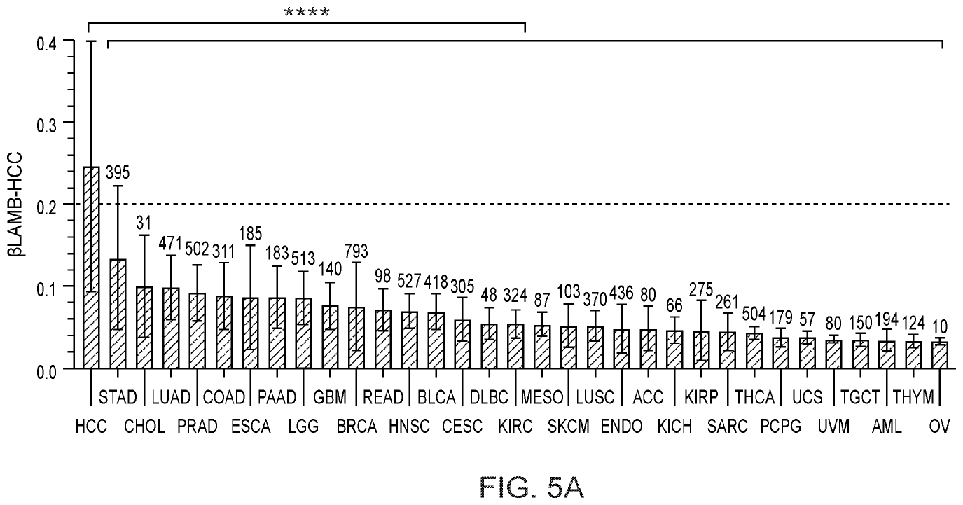

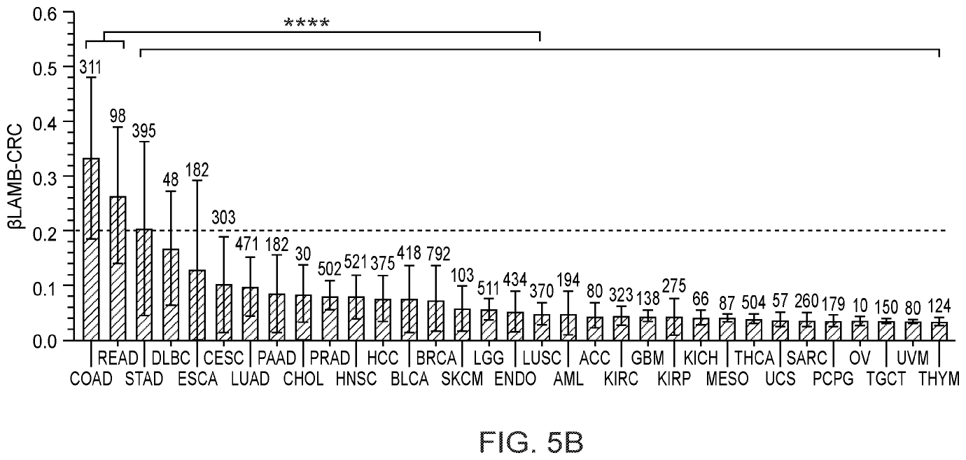

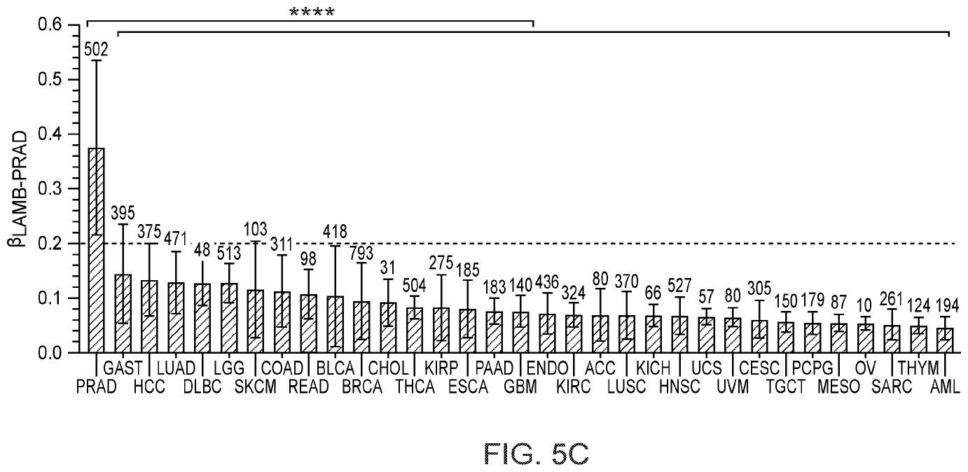

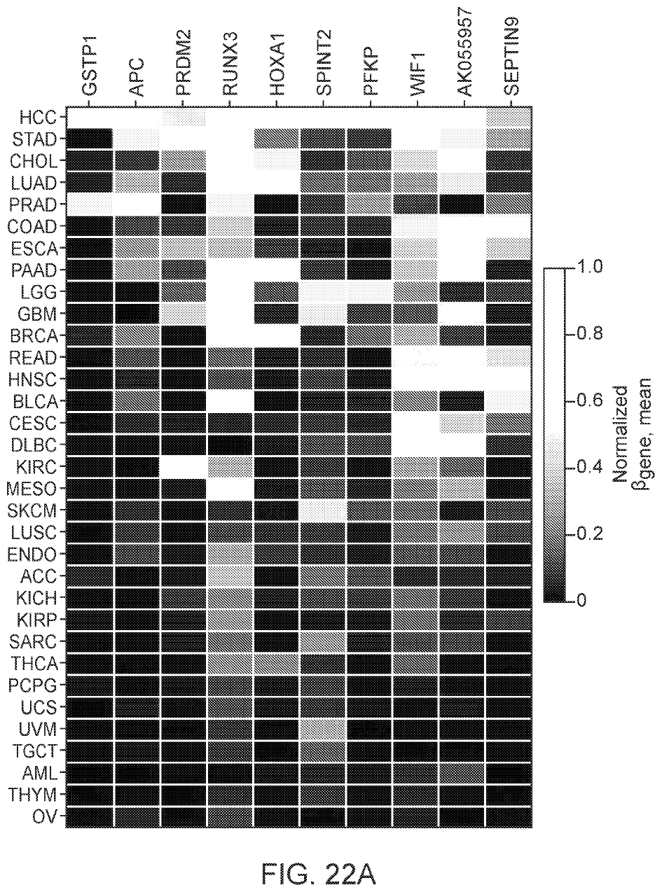

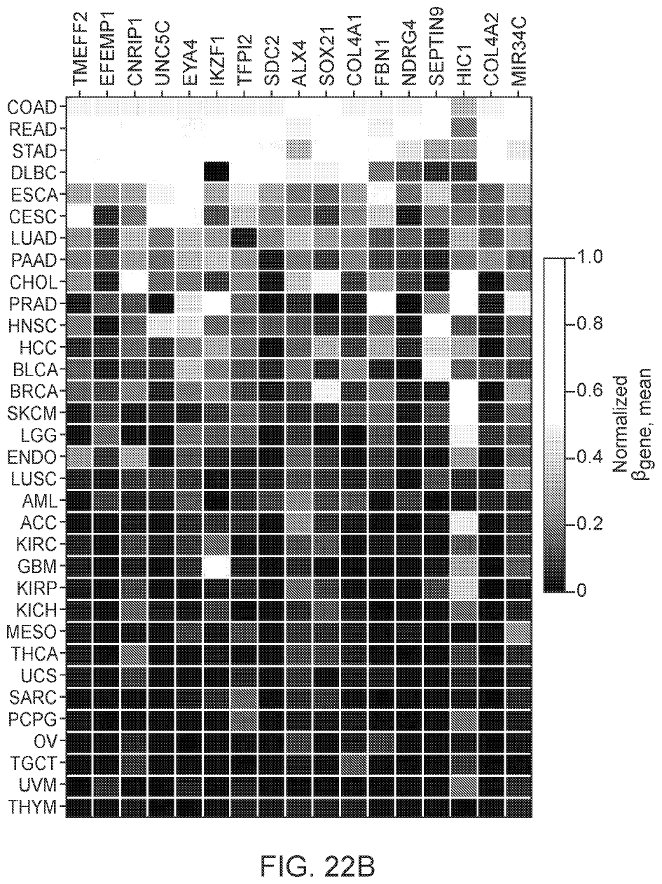

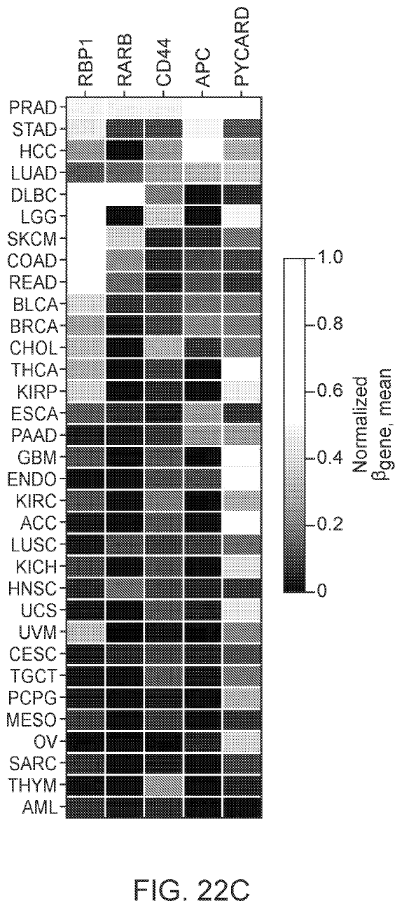

To assess the LAMB panels' specificity to their respective cancer types, we first examined the methylation frequencies of the panel's genes in 7287 tumors from 30 other cancer types from TCGA (50). The majority of genes in the panels were more methylated in another cancer type, but no other cancer type was hypermethylated for genes in all panels (β_{gene}>0.2; FIG. 22). This latter trend was also seen in the tumors' β_{LAMB} scores, with 2.2%, 6.8%, and 5.4% of tumors of other cancer types being hypermethylated for the LAMB-HCC, LAMB-CRC, and LAMB-PRAD panels, respectively (FIGS. 5A, 5B, and 5C). LAMB panel hypermethylation was specific to their respective tumor type (p<0.0001 for β_{LAMB-HCC} in HCC vs. other cancer types; p<0.0001 for β_{LAMB-CRC} in CRC vs. other cancer types; and p<0.0001 for β_{LAMB-PRAD} in PRAD vs. other cancer types), although individual genes in the LAMB panels are hypermethylated in tumors of other cancer types.

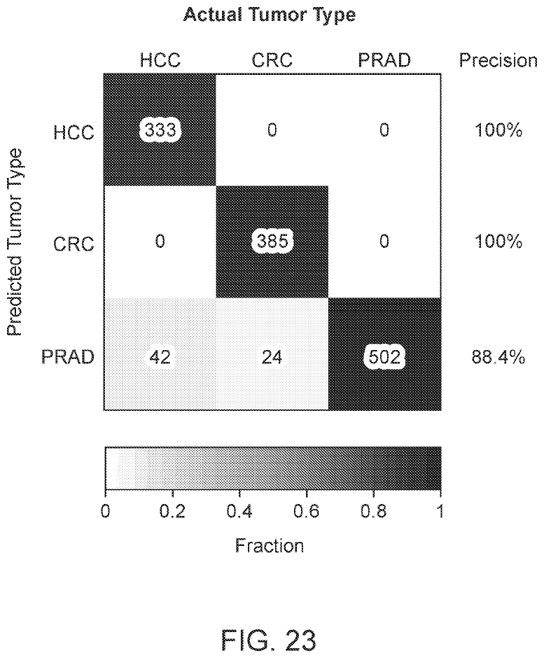

We lastly tested the LAMB panels' ability to classify cancer type of HCC, CRC, and PRAD tumors from TCGA. β_{LAMB-HCC}, β_{LAMB-CRC}, β_{LAMB-PRAD} scores for 375 HCC, 409 CRC, and 502 PRAD tumors were determined. By predicting cancer type through the highest methylation score of the LAMB panels, 95% of tumors were correctly classified, highlighting the panels' specificity to their tumor type (FIG. 23).

### DISCUSSION

In this study, we developed a methylated cfDNA cancer biomarker discovery method that integrates tumor suppressor methylation meta-analysis data with CpG methylation microarray data from cancer tissue, ANT, and healthy control blood (FIG. 1A). Our method seeks to improve upon previous methylated cfDNA biomarker discovery approaches by screening CpGs for their ability to not only differentiate cancer tissues and healthy blood, but also differentiate cancer tissues and ANTs. We have designed LAMB to minimize the detection of correlative CpG biomarkers by first identifying a small pool of promising gene biomarkers through LAMB Layer 1 and then limiting our CpG biomarker pool to CpGs in the promoter regions of these genes. AUC metrics in LAMB Layers 2 and 3 select high-sensitivity CpGs, and methylation frequency metrics for ANT and blood in these same layers select high-specificity CpGs. As a result, LAMB evaluates biomarkers across multiple data types, sample types, and patient cohorts in an effort to capture the epigenetic diversity of a cancer type and discover high-sensitivity, high-specificity biomarkers for that cancer.

This study used external cfDNA validation data to show that LAMB filtered out inaccurate CpG biomarkers and discovered methylated cfDNA panels that exhibited high predictive power for detecting HCC tumors in at-risk liver cirrhosis patients, detecting CRC tumors in average-risk patients, and differentiating PRAD response to therapy (FIGS. 2C and 3B). Geometric mean scores allowed us to test the predictive power of entire LAMB panels and individual LAMB genes in an unbiased manner with the cfDNA data. The high predictive power shown by these panels could be attributed to the functional diversity of the tumor suppressors within these panels and the panels' hypermethylation in the majority of early-stage and late-stage tumors (FIGS. 12 to 14).

Some genes in the panels have previously been tested in plasma to detect tumors in at-risk patients; however, these genes typically showed low accuracy in these studies (51-57). The higher predictive power seen in LAMB panels may be due to the inclusion of more genes or improvements in the resolution of methylation frequency quantification provided by microarrays and sequencing; other studies largely used binary or semi-quantitative PCR-based methods. Poorer performance in these studies may also be due to the studies including promoter CpGs that are not hypermethylated in cancer tissue and hypomethylated in ANT and blood. This is the case in the HCC meta-analysis panel, which contains all of the CpGs in the promoter areas of the genes identified by LAMB Layer 1. As a result, this panel contained both predictive and non-predictive CpGs and thus demonstrated lower predictive power than the LAMB-HCC panel (FIG. 15 and FIG. 2C). These results highlight the role of LAMB Layers 2 and 3 for removing CpGs that are not predictive for cancer detection.

In a similar way, the lower predictive power seen in the HCC and PRAD microarray panels highlights the value of LAMB Layer 1. Applying Layers 2 and 3 to all promoter CpGs in the 450K microarray to discover the microarray panels is akin to data-driven methods that have a significant imbalance between biomarker pool size and patient cohort size; although, it is crucial to note that the LAMB microarray discovery approach can still be considered knowledge-based because it only screens CpGs in promoter regions (unlike other published data-driven methods). Still, HCC and PRAD microarray panels showed lower predictive power than their respective LAMB panels, and LAMB panel CpGs exhibited higher median AUC than the microarray panel CpGs, highlighting that a portion of the CpGs in the microarray panels are not representative of their respective cancer type in tumors outside those in the discovery microarray datasets (FIGS. 2C, 3B, 16 and 20). By identifying a smaller pool of candidate tumor suppressors with meta-analysis data, LAMB Layer 1 improves the final panel's predictive power by decreasing the likelihood that Layers 2 and 3 select correlative CpGs. The significantly smaller size of LAMB-HCC and LAMB-PRAD in comparison to the meta-analysis and microarray panels (21 CpGs vs. 309/486 CpGs and 19 CpGs vs. 123/1178 CpGs, respectively) is an added benefit of beginning with a candidate tumor suppressor gene pool. A biomarker panel's size is critical for its clinical adoption, as smaller panels can increase sample throughput and decrease assay cost.

We also observed that LAMB biomarker panels demonstrated higher predictive power and tumor-specificity than individual LAMB genes (FIGS. 2C, 3 and 4B; Tables 1 to 3). Layers 2 and 3 select CpGs that are differentially methylated in a large set of cancerous tissues, ANTs, and healthy blood samples. This results in a panel of CpGs that is consistently co-hypermethylated in cancerous tissue and co-hypomethylated in ANT and blood. Accordingly, the panel's combined methylation frequency represents a tumor-specific methylation signal in the discovery set of tissues and blood samples. The low-dimensional tissue and blood microarray data selected by LAMB Layer 1 seems to be representative of methylation trends in HCC, CRC, PRAD, healthy liver, healthy colorectal, healthy prostate, and blood samples, as the panels' tumor-specific signals were seen through their hypermethylation in the tissue datasets of their respective cancer types and their hypomethylation in the tissue datasets of other cancer types (FIGS. 2C, 4C, and 4D). This tumor-specificity is key for detecting a tumor's tissue-of-origin.

This study used geometric mean scores to assess the predictive power of LAMB biomarker panels in an unbiased manner due to the smaller patient cohorts in the cfDNA validation data. As a result, the purpose of this study was defined to creating the LAMB method, testing its biomarker selection ability, and providing early data on the predictive power of biomarker panels discovered through LAMB. LAMB-HCC, LAMB-CRC and LAMB-PRAD have the potential to fulfill critical clinical needs in cancer detection and therapy monitoring. To further validate these panels, larger clinical studies are required, in which classification models fitted to LAMB gene biomarkers and complementary protein biomarkers like AFP and PSA could improve the panels' predictive power.

LAMB could also be used to discover methylated cfDNA biomarker panels for other cancer types. Here, we applied LAMB to HCC, CRC, and PRAD, but our method is specifically designed to be applied to any cancer type and to leverage the extensive amount of public cancer methylation data available today. Thousands of tissue methylation studies have been published in the past two decades, and hundreds of 450K datasets of cancer tissues, ANTs, and blood have been created and shared by TCGA and other research groups. These studies and datasets can together be analyzed through LAMB to discover additional cancer biomarkers for blood-based applications.

All in all, this study introduced LAMB, a knowledge-based methylated cfDNA biomarker discovery method that integrates meta-analysis and microarray data to discover predictive cancer biomarker panels, and provided early data supporting its ability to discover biomarker panels that can improve cancer diagnosis and therapy monitoring. The HCC, CRC, and PRAD LAMB panels in this study exhibit a tumor-specific methylation signature in tissue and cfDNA validation data. Future steps include further validating these panels in large-scale studies and applying LAMB to discover methylated cfDNA biomarker panels for other cancer types.

### Methods

### Study Design

This study aimed to show that targeted panels of predictive methylated cfDNA biomarkers for cancer could be found through a knowledge-based analysis of published studies and microarray datasets. To this end, we first conducted a meta-analysis of tumor suppressor methylation studies for HCC, CRC, and PRAD, using the results to find candidate gene biomarkers. We then screened CpGs in the promoter regions of these genes with microarray data of malignant tissue, paired ANT, and healthy blood from thousands of patients to find biomarkers that demonstrated high-sensitivity and high-specificity through control tissue methylation frequency and AUC metrics. Genes in the panels were examined for cross-interactions and shared molecular pathways. Then, these panels were tested in tissue and cfDNA validation datasets. 450K microarray datasets for HCC and PRAD cfDNA were used to test LAMB's selection capabilities for diagnostic and prognostic biomarkers. Through unbiased geometric mean scores, LAMB-HCC was evaluated for detection of HCCs in cfDNA of at-risk cirrhosis patients, LAMB-CRC was assessed for detection of CRCs in cfDNA of average-risk patients, and LAMB-PRAD was assessed for distinguishing tumor response to AA and docetaxel by AUC and p-value metrics. Authors were blinded to sample information from the cfDNA data when finding LAMB-HCC, LAMB-CRC, and LAMB-PRAD gene and panel scores. We finally assessed the panels' specificity to their respective cancer types using their methylation frequencies in tumors of other cancer types.

### LAMB Biomarker Panel Discovery Process

LAMB is comprised of three selection layers (FIG. 1A). Layer 1 identified candidate tumor suppressor genes through count data of paired cancerous tissues and ANTs from published studies. Methylated cancerous tissues and unmethylated ANTs were categorized as true positives and true negatives, respectively. A random-effects meta-analysis was then used to calculate the diagnostic odds ratios (DORs) for tumor suppressors in multiple studies. Genes with a statistically significant DOR (DOR_{95% CI}>1) were picked for further analysis. For HCC, eight tumor suppressors showing high predictive power for differentiating cancer patients from cirrhosis patients by cfDNA through fitted classification models were also chosen for further analysis (AUC>0.8).

Layer 2 filtered CpGs in the promoter areas of candidate tumor suppressors from Layer 1 by methylation frequency (0). 450K data of paired cancer and ANT tissues from TCGA and other public sources were combined, batch-adjusted, and subsetted for CpGs in the promoter regions of tumor suppressors that survived Layer 1. CpGs that were more methylated in cancer tissues than ANTs (Δβₘₑₐₙ>0), hypomethylated in ANTs (β_{ANT}<0.2), and showed a high predictive power for differentiating cancer tissues and ANTs (AUC>0.8) were selected for further analysis.

Layer 3 filtered surviving CpGs in 1722 lysed whole blood samples from healthy controls compiled from public 450K datasets. To select biomarkers distinguishable from healthy cfDNA, Layer 3 first selected CpGs with high predictive power for differentiating cancer tissues and lysed whole blood samples that were matched by age, gender, and race (AUC>0.8). Tissue and blood samples were chosen without any bias from other clinical, demographic, or methylation frequency factors. From this filtered set of CpGs, Layer 3 then selected CpGs demonstrating low background methylation in unmatched blood samples (βₘₑₐₙ<0.1).

LAMB biomarker panels were made up of CpGs that survived filtering from LAMB Layers 1, 2, and 3. We used the LAMB biomarker discovery process with meta-analysis and microarray data for HCC, CRC, PRAD, NSCLC, and BRCA to discover the LAMB-HCC, LAMB-CRC and LAMB-PRAD, LAMB-NSCLC and LAMB-BRCA panels.

### Protein-Protein Interaction Network (PPIN) and Pathway Enrichment Analysis

To assess the functional diversity of genes in the LAMB panels, we input the panels' sets of genes into STRING, a database of known and predicted protein-protein interactions (58). PPINs with edges representing data support confidence and interaction evidence were created using experimental, neighborhood, gene fusion, co-occurrence, and co-expression information with a minimum required interaction score of 0.40. To discover molecular pathways shared by the panels' genes, gene sets were queried in the g:Profiler database (59).

### LAMB Biomarker Evaluation Through Geometric Mean Scores

Due to the small sizes of the patient cohorts, prediction models fitted to LAMB biomarkers were not employed to avoid potential overfitting. To assess the predictive power of the biomarker panels and genes in an unbiased manner, we used the geometric mean. We first calculated the gene methylation frequency, β_{gene}, for all genes in a panel using the geometric mean of the methylation frequencies for CpGs in the panel that are in the promoter area of the gene (β_{CpG}):

$${β}_{gene} = \sqrt[{n}_{CpG}]{{\prod }_{i = 1}^{{n}_{CpG}} {β}_{CpG , i}} ,$$

where n_{CpG} is the number of CpGs in the panel that are in the promoter area of the gene and β_{CpG, i} is the methylation frequency of the i-th CpG in the panel that is in the promoter area of the gene for that sample. We then found the panel's methylation frequency, βₚₐₙₑₗ, for each sample by taking the geometric mean of the methylation frequencies of genes in the panel:

$${β}_{panel} = \sqrt[{n}_{gene}]{{\prod }_{i = 1}^{{n}_{gene}} {β}_{gene , i}} ,$$

where n_{gene} is the number of genes in the panel and β_{gene, i} is the methylation frequency of the i-th gene in the panel for that sample. Frequencies input to the geometric mean are normalized so that they are equally weighted in the output. Accordingly, CpG and gene methylation frequencies are equally weighted in β_{gene} and βₚₐₙₑₗ, respectively, resulting in unbiased outputs. We calculated β_{gene} and βₚₐₙₑₗ for the LAMB-HCC, LAMB-CRC, LAMB-PRAD, meta-analysis, and microarray panels.

### Tissue Analysis

450K microarray data for 8573 primary tumors comprising HCC tumors, CRC tumors, PRAD tumors, primary tumors for 30 other cancer types, HCC ANT, CRC ANT, and PRAD ANT was downloaded from TCGA (50). A summary of the 30 other types of cancer is in the Supplementary Methods and Materials. β_{gene} and βₚₐₙₑₗ scores for LAMB-HCC, LAMB-CRC and LAMB-PRAD panels were found for the samples. Primary Gleason grade and secondary Gleason grade were combined to obtain Gleason scores for PRADs. HCC/CRC tissues and PRAD tissues were separated by tumor stage and Gleason score, respectively; β_{LAMB-HCC}/β_{LAMB-CRC} and β_{LAMB-PRAD} scores were compared across the categories. HCC tumors, CRC tumors, and PRAD tumors were also compared against the other types of cancer.

HCC and Cirrhosis cfDNA Analysis

450K microarray data for HCC and cirrhosis cfDNA was downloaded from GSE129374. Methylation frequencies for CpGs in the LAMB-HCC, meta-analysis, and microarray panels were extracted, and β_{gene} and βₚₐₙₑₗ scores for all panels were calculated. Patient information for the samples was then used to match the 44 patients to disease state. For the combined AFP and LAMB-HCC analysis, 13 HCC patients with AFP values above 20 ng/mL and 3 cirrhosis patients without AFP values were removed from the dataset.

CRC cfDNA Analysis

Targeted bisulfite sequencing data for CRC and healthy control cfDNA was downloaded from GSE149438. LAMB-CRC gene regions were defined as containing LAMB-CRC CpGs for that gene and CpGs between the LAMB-CRC CpGs. Flanking 25 bp segments were added to 12 gene regions so that their β_{gene} scores had a resolution of at least 0.1% (i.e., for these gene regions: methylated CpG count+unmethylated CpG count≥1000). Methylated and unmethylated CpG counts were extracted for these gene regions, and β_{gene} and β_{LAMB-CRC} scores were found. Patient information for the samples was then used to match the 84 patients to disease state and tumor stage.

PRAD cfDNA Analysis

Raw 450K microarray data for PRAD cfDNA samples was downloaded from GSE108462. Methylation frequencies for CpGs in the LAMB-PRAD, PRAD meta-analysis, and PRAD microarray panels were extracted, and β_{gene} and βₚₐₙₑₗ scores for all the panels were calculated. Patient, PSA, and day information for these samples was matched to the methylation data of the 23 patients treated with abiraterone acetate and docetaxel. A few visits had multiple technical replicates. Visits with technical replicates exhibiting a coefficient of variation greater than 20% in β_{LAMB-PRAD} score were removed; otherwise, the geometric mean of the methylation frequencies for the visit's technical replicates was mapped to the visit.

For on-treatment samples in patients, percent difference scores from the patient's baseline were calculated for β_{CpG}, β_{gene}, and βₚₐₙₑₗ values. Patients were categorized as responders based on the PSA change from their baseline pre-treatment sample to the first on-treatment sample. 9 patients with a PSA decrease greater than 30% were classified as responders with the remaining 14 patients classified as non-responders. After the first on-treatment visit, tumor responses were classified by the visit's PSA value in relation to the past visit's PSA value, the patient's nadir PSA value at that point in treatment, and the PSA clinical cut-off for a serologically abnormal value (4 ng/mL). Visits with PSAs that were less than the nadir, the past visit, or the cut-off were classified as time points when the tumor was responding, and all other visits were classified as time points when the tumor was resistant.

### Statistical Analysis

As mentioned before, the geometric mean was used to calculate gene and panel methylation scores for the LAMB, meta-analysis, and microarray panels. The scores were evaluated in cfDNA data with ROC curves and one-sided Mann-Whitney tests since the Shapiro-Wilk test showed that these scores were not normally distributed. For this reason, two-sided Mann-Whitney tests were used to compare LAMB panel scores among HCC, CRC, and PRAD tumor stages as well as other cancer types, and one-sided Mann-Whitney tests were used to compare individual CpG AUCs in different panels. 95% CIs for AUC values of the panel scores were found with the Wilson/Brown method. For the LAMB biomarker discovery method, adjusted DORs and 95% CIs were calculated using the Hartung-Knapp-Sidik-Jonkman estimator. As mentioned before, AUC and β_{CpG} values were used in LAMB to screen CpG biomarkers. βₘₑₐₙ values for malignant tissue, ANT, and whole blood were found using the geometric mean. All statistical analysis was conducted in R and Prism.

### Supplementary Materials and Methods

### LAMB Layer 1: Gene Meta-Analysis of HCC Tissue Methylation Studies

A search in both PubMed and Embase for “((((hepatocellular carcinoma) OR HCC) OR hepatoma) OR hepatocarcinoma) AND methylation” led to 2805 records (as of Oct. 1, 2019). These records were screened for relevance to HCC tissue methylation, resulting in 637 pertinent tissue methylation papers (FIG. 6). Papers were screened for methylation frequency data, reducing inclusion to 317 studies. Remaining papers were also filtered for testing HCC and adjacent non-cancerous tissue (ANT) from the same patients and the use of methylation-specific PCR (MSP) or quantitative MSP (QMSP) for methylation quantification, further reducing inclusion to 123 papers. Count data was collated for 67 papers that tested genes that were also analyzed in at least two other studies. For this count data, hypermethylated promoters for paired HCCs and ANTs were regarded as true positives and false positives, respectively. In the “metafor” library in R, a random-effects meta-analysis using the Hartung-Knapp-Sidik-Jonkman estimator was conducted to find adjusted diagnostic odds ratios (DOR) with 95% confidence intervals (CI) (60-61). Genes with DOR 95% CI less than 1 were discarded. Forest plots were created for remaining genes. Plasma methylation studies of HCC and cirrhosis patients were identified during this search and examined for genes showing high predictive ability in fitted classification models (area under curve, AUC>0.8) (64-65).

### LAMB Layer 2: Promoter CpG Methylation in HCC and ANT Microarrays

Illumina HumanMethylation450 (450K) methylation frequency data was downloaded from The Cancer Genome Atlas's study on Liver Hepatocellular Carcinoma (TCGA-LIHC). Recurrent tumors were removed and data for 50 patients with paired primary HCCs and ANTs were used. 450K data for 66 patients (GSE54503) and 37 patients (GSE89852) with paired malignant/ANT tissues were downloaded from the Gene Expression Omnibus (GEO). These datasets were selected because they had public demographic information, which can be found in their associated studies. All three datasets were combined into a single dataset, and any CpG that was missing in a sample was removed, leading to 326,322 CpGs. The data was logit-transformed, the “ComBat” package in R was used to correct for batch effects among the datasets, and the data was then inverse logit-transformed (62). The resulting CpGs were then annotated with their genes, features, and chromosomal location information with Illumina's 450K annotation file. Methylation frequency data for the CpGs in the TSS1500 and TSS200 regions of the 23 genes from the tissue meta-analysis and plasma study analysis was extracted for all 153 patients. From the 153 patients, methylation frequency data for a cohort of 67 patients (TCGA and GSE89852) with primarily early-stage tumors was extracted to evaluate the sites' performance in early-stage tumors. With the R “pROC” library, geometric mean HCC methylation, geometric mean ANT methylation, and univariate CpG AUCs between HCCs and ANTs were calculated for all patients and the early-stage sub-cohort (63). CpGs with a lower mean methylation in HCC than ANT, hypermethylated in ANT (methylation frequency: β>0.2), or with AUC between malignant tissue and ANT less than 0.8 were removed.

### LAMB Layer 3: Promoter CpG Methylation in HCC and Whole Blood

450K methylation frequency data of lysed whole blood was downloaded for 305, 127, 622, 272, 236, and 160 healthy patients from GEO (GSE84727, GSE80417, GSE40279, GSE72773, GSE111629, GSE53740) (93-97). These datasets were selected because they analyzed lysed whole blood and were structured such that healthy controls were identifiable. CpGs that were not in all patients in all the datasets were removed, and the resulting data was combined into a methylation frequency matrix and logit-transformed. The data was then corrected for batch effects. Methylation frequency data for CpGs found in LAMB Layer 2 was extracted from the blood data. To perform a differential methylation analysis of HCC tissue and blood, 450K data for 159 other TCGA HCCs was downloaded. These tumors were matched to 159 of the whole blood samples from the healthy control patients based on patient age, gender, and race. The ratio of matched blood samples by their GEO dataset mirrored the ratio of total blood samples by their GEO dataset. CpGs with AUCs less than 0.8 between HCC tissue and control blood were removed. The geometric mean methylation frequencies of the remaining CpGs in the remaining 1563 blood samples were calculated. CpGs with a methylation frequency lower than 0.1 in the unpaired blood samples were selected as CpGs for the LAMB-HCC panel.

### Validation: LAMB-HCC Panel Analysis in Cell-Free DNA

450K methylation data was downloaded for 22 cirrhosis with HCC and 22 cirrhosis without HCC patients (GSE129374); these patients were matched by their liver function. As of May 2020, GSE129374 is the only methylation frequency data available with cfDNA from HCC and cirrhosis patients. LAMB-HCC CpG data was extracted from cfDNA data; the geometric mean frequency of the LAMB CpGs within a gene promoter was mapped to that gene to create 10-gene methylation profiles for all 44 patients. The geometric mean of each of the 10-gene methylation profiles was calculated to find the LAMB-HCC score. The patients' disease states were mapped to each patient methylation profile. A ROC curve was created for LAMB-HCC scores, and AUCs were calculated for gene and LAMB-HCC scores. AUCs were then calculated for all CpGs tested by LAMB's 450K filters. AUC 95% CI for the LAMB-HCC scores were determined through the Wilson/Brown method. A Wilcoxon test was used to find statistical significance.

To test the LAMB panel's use of meta-analysis and microarray data, HCC panels found solely with meta-analysis and microarray data were tested. For the HCC meta-analysis panel, all CpGs in the cfDNA data that are in the promoter regions of the 22 genes identified with the meta-analysis and literature search were included in the panel, totaling 364 CpGs. This panel's CpG methylation data was extracted from the cfDNA data, and the geometric mean frequency of CpGs in a gene promoter was mapped to that gene to create 22-gene methylation profiles for all patients. A ROC curve was created, and AUCs were found from individual CpGs as well as meta-analysis scores derived from the geometric mean of this 22-gene methylation profile. A Wilcoxon test was used to calculate statistical significance. For the HCC microarray panel, 326,322 CpGs present in all patient samples from LAMB-HCC Layer 2 input data were screened to find those that were in gene promoter areas and then filtered with LAMB Layers 2 and 3, resulting in a panel containing 486 CpGs with 267 genes. This panel's CpG methylation data was extracted from the cfDNA data, and the geometric mean frequency of CpGs in a gene promoter was mapped to that gene to create 267-gene methylation profiles for all 44 patients. A ROC curve was created, and AUCs were found for individual CpGs as well as microarray scores calculated with the geometric mean of this 267-gene methylation profile. A Wilcoxon test was used to determine statistical significance.

Alpha-fetoprotein (AFP) serum levels were found for 41 patients in the 450K cfDNA validation dataset from the paper's supplement and matched to the patients based on data provided from the study's authors. AFP levels of 3 out of 22 cirrhosis patients were not available, so they were removed from this analysis. For the remaining patients, 9 out of 22 HCC and all 19 cirrhosis patients had AFP levels below the clinical cutoff of 20 ng/mL. The LAMB-HCC panel scores for these 9 HCC patients and 19 cirrhosis patients were analyzed. A ROC curve was created from this data, and an AUC was found with 95% CI calculated using the Wilson/Brown method. A Wilcoxon test was used to determine statistical significance.

### LAMB-CRC (Colorectal Adenocarcinoma):

### LAMB Layer 1: Gene Meta-Analysis of Colorectal Adenocarcinoma (CRC) Tissue Methylation Studies

A search in both PubMed and Embase for “(colorectal adenocarcinoma) OR CRC) OR colon adenocarcinoma) OR rectal adenocarcinoma) OR colorectal carcinoma) OR colon carcinoma) OR rectal carcinoma) OR colorectal cancer) AND methylation” led to 6400 abstracts (as of Jul. 1, 2020). These abstracts were screened for relevance to CRC tissue methylation and duplication, resulting in 2129 pertinent tissue methylation papers (FIG. 12). Papers were screened for methylation frequency data, reducing inclusion to 916 studies. Remaining papers were also filtered for testing CRC and ANT from the same patients and use of MSP or QMSP for methylation quantification, further reducing inclusion to 400 papers. Count data was collated for 188 papers that tested genes that were also analyzed in another study. Hypermethylated promoters for paired CRCs and ANTs were true positives and false positives, respectively. A random-effects meta-analysis using the Hartung-Knapp-Sidik-Jonkman estimator was done to find adjusted diagnostic odds ratios (DOR) with 95% confidence intervals (CI). Genes with DOR 95% CI less than 1 were discarded. Forest plots were created for remaining genes.

### LAMB Layer 2: Promoter CpG Methylation in CRC and ANT Microarrays

Illumina HumanMethylation450 (450K) methylation frequency data was downloaded from TCGA's studies on Colon Adenocarcinoma and Rectal Adenocarcinoma (TCGA-COAD, TCGA-READ). Recurrent tumors were removed and data for 44 patients with paired primary CRCs and ANTs were used. 450K data for 95 patients (GSE 77718) and 103 patients (GSE 101764) with paired malignant/ANT tissues were downloaded from the Gene Expression Omnibus (GEO). These datasets were selected because they had public demographic information, which can be found in their associated studies. All three datasets were combined into a single dataset, and CpGs that were missing in a sample was removed, leading to 300,885 CpGs. Data was logit-transformed, ComBat was used to correct for batch effects among the datasets, and data was then inverse logit-transformed. The resulting CpGs were annotated with their genes, features, and chromosomal location information with Illumina's 450K annotation file. Methylation frequency data for CpGs in the TSS1500 and TSS200 areas of the 75 genes from the tissue meta-analysis was extracted for all patients. Geometric mean CRC methylation, geometric mean ANT methylation, and univariate CpG AUCs between CRCs and ANTs were calculated for all patients. CpGs with a lower mean methylation in CRC than ANT, hypermethylated in ANT (β>0.2), or with AUC between malignant tissue and ANT less than 0.8 were removed.

### LAMB Layer 3: Promoter CpG Methylation in CRC and Whole Blood

450K data of whole blood from the HCC LAMB Layer 3 analysis was utilized. Methylation frequency data for CpGs identified in CRC LAMB Layer 2 was extracted from the blood data. To perform a differential methylation analysis of CRC tissue to blood, 450K data for 283 separate TCGA CRCs was downloaded. Tumors were matched to 283 of the whole blood samples from the healthy control patients based on patient age, gender, and race. The ratio of matched blood samples by GEO dataset mirrored the ratio of total blood samples by GEO dataset. CpGs with AUC<0.8 between CRC tissue and whole blood were removed. Geometric mean methylation frequencies of remaining CpGs in the remaining 1439 blood samples were calculated. CpGs with a methylation frequency less than 0.1 in these unpaired blood samples were identified as LAMB CpGs for CRC.

### Validation: LAMB-CRC Panel Analysis in Cell-Free DNA

Targeted bisulfite sequencing data was downloaded for 38 CRC patients and 46 healthy controls matched by age (GSE149438). As of October 2020, this is the only methylation frequency data available with cfDNA from CRC and average-risk patients. LAMB-CRC gene regions were defined as containing LAMB-CRC CpGs for that gene and CpGs between LAMB-CRC CpGs. Flanking 25 bp segments were added to the gene regions so that every β_{gene} score had a resolution of at least 0.1%. Five of the LAMB-CRC genes had dramatically lower sequencing depth and were removed from analysis. Methylated and unmethylated CpG counts were extracted for gene regions, and β_{gene} and β_{LAMB-CRC} scores were found. Patient information for these samples was used to match patients to disease state and tumor stage. A ROC curve was created for LAMB-CRC scores, and AUCs were calculated for gene and LAMB-CRC scores. AUC 95% CI for the LAMB-CRC scores were determined through the Wilson/Brown method. A Wilcoxon test was used to find statistical significance.

### LAMB-PRAD (Prostate Adenocarcinoma):

### LAMB Layer 1: Gene Meta-Analysis of Prostate Adenocarcinoma (PRAD) Tissue Methylation Studies

A search in PubMed and Embase for “((((prostate adenocarcinoma) OR prostate cancer) OR prostate carcinoma) OR PRAD) AND methylation” led to 3775 abstracts (as of Jan. 1, 2020). These abstracts were screened for relevance to PRAD tissue methylation and duplication, resulting in 669 pertinent tissue methylation papers (FIG. 12). Papers were screened for methylation frequency data, reducing inclusion to 369 studies. The remaining papers were screened for having ANTs from the PRAD patients as control tissues and use of MSP/QMSP for methylation frequency quantification, further reducing inclusion to 105 papers. Count data was collated for the 41 papers that contained genes that were tested in two or more studies. Hypermethylated promoters for paired PRADs and ANTs were classified as true positives and false positives, respectively. A random-effects meta-analysis using the Hartung-Knapp-Sidik-Jonkman method was conducted, resulting in adjusted diagnostic odds ratios (DOR) with 95% CI. Genes with DOR 95% CI less than 1 were discarded. Forest plots were then created for remaining genes.

### LAMB Layer 2: Promoter CpG Methylation in PRAD and ANT Microarrays

450K data was downloaded from The Cancer Genome Atlas for PRAD (TCGA-PRAD). Recurrent tumors were removed and data for 50 patients with paired primary PRADs and ANTs was used. 450K data for 95 additional PRAD patients with paired tissues was downloaded from GEO (GSE112047, GSE55598, GSE73549, GSE76938). All five datasets were concatenated into a single dataset, and any CpG that was missing in a sample was removed, leading to 229,815 CpGs for 145 patients. The data was logit-transformed, corrected for batch effects among datasets, and the data was inverse logit-transformed. The remaining CpGs were coupled to genes, features, and chromosomal locations in Illumina's 450K annotation file. Methylation frequency data for the CpGs in the TSS1500 and TSS200 of the 8 genes from the meta-analysis was extracted for all patients. Geometric mean PRAD methylation, geometric mean ANT methylation, and univariate CpG AUCs between PRADs and ANTs were calculated for the 145 patients. Due to the small number of paired patients (2) with early-stage PRADs, additional early-stage AUC analysis was not conducted. CpGs with a lower mean methylation in PRAD than ANT, hypermethylated in ANT (methylation frequency: β>0.2), or an AUC less than 0.8 for distinguishing PRAD and ANT were removed.

### LAMB Layer 3: Promoter CpG Methylation in PRAD and Whole Blood

450K data of lysed whole blood from the HCC LAMB Layer 3 analysis was utilized. Methylation frequency data for CpGs identified in PRAD LAMB Layer 2 was extracted from the blood data. To perform a differential methylation analysis of PRAD tissue to blood, 450K data for 315 separate TCGA PRADs was downloaded. These tumors were matched to 315 of the whole blood samples from the healthy control patients based on patient age, gender, and race. The ratio of matched blood samples by their GEO dataset mirrored the ratio of total blood samples by their GEO dataset. CpGs with an AUC<0.8 between PRAD tissue and whole blood were removed. Geometric mean methylation frequencies of remaining CpGs in the remaining 1407 blood samples were calculated. CpGs with a methylation frequency less than 0.1 in these unpaired blood samples were identified as LAMB CpGs.

### Validation: LAMB-PRAD Panel Analysis in Cell-Free DNA

450K methylation data was downloaded for all visits of castration-resistant prostate cancer (CRPC) patients (GSE108462). As of May 2020, GSE108462 is the only methylation frequency data available for cell-free DNA for CRPC patients. LAMB-PRAD CpG data was extracted from the cfDNA validation data; the geometric mean frequency of the LAMB CpGs in a gene promoter was mapped to that gene to create 5-gene methylation profiles for all visits. The geometric mean of each of the 5-gene methylation profiles was calculated to find the LAMB-PRAD score. Patient, PSA and day information were mapped to the visits' methylation profiles. Coefficients of variation for the LAMB-PRAD scores were found for visits with multiple technical replicates; visits with coefficients of variation greater than 20% were removed. The geometric mean of the methylation frequencies for the remaining visits with technical replicates were mapped to that visit. 23 patients treated with abiraterone acetate and docetaxel with day 0, pre-treatment samples were analyzed. For on-treatment samples for each patient, percent difference scores from the patient's baseline were calculated. Patients were categorized as responders based on the PSA change from their baseline sample to first on-treatment sample. Nine patients with a PSA decrease greater than 30% were classified as responders with the remaining 14 patients classified as non-responders. A ROC curve to differentiate responders and non-responders by their percent difference in LAMB-PRAD scores from baseline to first visit was created, and AUCs were calculated for first-visit gene and LAMB-PRAD percent difference scores. AUCs were calculated for first-visit percent difference scores for PRAD CpGs evaluated by LAMB Layers 2 and 3. AUC 95% CI for the LAMB-PRAD scores was calculated with the Wilson/Brown method. A Wilcoxon test was used to determine statistical significance.

To evaluate LAMB-PRAD's use of both meta-analysis and microarray data, PRAD panels found only through meta-analysis and microarray data were assessed. For the PRAD meta-analysis panel, CpGs in the cfDNA data that are in the promoter regions of the nine genes identified in the meta-analysis were included in the panel, totaling 131 CpGs. This panel's CpG methylation data was extracted from the cfDNA data for visits used in the LAMB-PRAD analysis. The geometric mean frequency of CpGs in a gene promoter was mapped to that gene to create 9-gene methylation profiles for all visits. The geometric mean of each of the 9-gene methylation profiles was calculated to find the PRAD meta-analysis panel score. For the first on-treatment sample for each patient, a percent difference score from the baseline visit was calculated with the PRAD meta-analysis panel scores. A ROC curve to differentiate responders and non-responders by these scores was created, and an AUC was calculated with 95% CI found with the Wilson/Brown method. A Wilcoxon test was used to calculate statistical significance. For a PRAD microarray panel, 229,815 CpGs present in all patient samples from LAMB-PRAD Layer 2 input data were screened to find those in gene promoter areas and then were filtered with LAMB Layers 2 and 3, resulting in 1178 CpGs in 624 genes. These CpGs' methylation data was extracted from the cfDNA data for visits in the LAMB-PRAD analysis. The geometric mean frequency of CpGs in a gene promoter region was mapped to that gene to create 624-gene methylation profiles for all visits. The geometric mean of each 624-gene methylation profile was calculated to find the PRAD microarray panel score. For the first on-treatment sample for each patient, a percent difference score from the baseline visit was calculated with the PRAD microarray panel scores. A ROC curve to separate responders and non-responders by these scores was created; an AUC was calculated with 95% CI found from the Wilson/Brown method. A Wilcoxon test was used to determine statistical significance.

To evaluate how LAMB-PRAD scores correlate with tumor progression and response after the first, on-treatment visit, tumor responses were classified by the visit's PSA value in relation to the past visit's PSA value, the patient's nadir PSA value at that point in treatment, and the PSA clinical cut-off for a serologically abnormal value (4 ng/mL). This classification was only done for visits for the 23 patients analyzed for LAMB-PRAD therapy response. Seven visits with PSAs that were less than the patient's PSA nadir, the past visit, or the PSA cut-off were categorized as time points when the tumor was responding, and the other 24 visits were classified as time points when the tumor was progressing. LAMB-PRAD scores for the 24 progressive visits were compared to the nadir LAMB-PRAD score at that point of treatment, while LAMB-PRAD scores for the seven response visits were compared to the peak LAMB-PRAD score at that point of treatment.

### LAMB Panels for NSCLC (Non-Small Cell Lung Cancer):

### LAMB Layer 1: Gene Meta-Analysis of Non-Small Cell Lung Cancer (NSCLC) Tissue Methylation Studies

A search in both PubMed and Embase for “(lung adenocarcinoma) OR LUAD) OR lung squamous cell carcinoma) OR LUSC) OR non-small cell lung cancer) OR NSCLC) AND methylation” was conducted on Oct. 1, 2019. These records were screened for relevance to NSCLC tissue methylation, for methylation frequency data, for testing NSCLC and ANT from the same patients, and for the use of MSP or QMSP for methylation quantification. Count data was collated for 88 papers. Hypermethylated promoters for paired NSCLCs and ANTs were regarded as true positive and false positive, respectively. A random-effects meta-analysis with the Hartung-Knapp-Sidik-Jonkman estimator was conducted to find adjusted diagnostic odds ratios (DOR) with 95% confidence intervals (CI). Genes with DOR 95% CI less than 1 were discarded. Plasma methylation studies of NSCLC and healthy patients were identified during this search and examined for genes showing high predictive ability in fitted classification models (area under curve, AUC>0.8).

### LAMB Layer 2: Promoter CpG Methylation in NSCLC and ANT Microarrays

Illumina HumanMethylation450 (450K) methylation frequency data was downloaded from The Cancer Genome Atlas's study on Lung Adenocarcinoma and Lung Squamous Cell Carcinoma (TCGA-LUAD, TCGA-LUSC). Recurrent tumors were removed and data for 23/40 patients with paired primary LUAD/LUSC and ANTs were used. 450K data for 15 patients (GSE75008), 12 patients (GSE83842), 8 patients (GSE85845), and 13 patients (GSE94785) with paired LUAD and ANT tissues were downloaded from GEO. 450K data for 16 patients (GSE75008) and 7 patients (GSE94785) with paired LUSC and ANT tissues were downloaded from GEO. The datasets were selected because they had public demographic information, which can be found in their associated studies. LUAD datasets were combined into a dataset, and LUSC datasets were combined into a dataset. Any CpG that was missing in a sample was removed. The datasets were logit-transformed, corrected for batch effects, and inverse logit-transformed. Resulting CpGs were annotated with their genes, features, and chromosomal location information with Illumina's 450K annotation file. Methylation frequency data for CpGs in the TSS1500 and TSS200 regions of the 48 genes from the tissue meta-analysis and plasma study analysis was extracted for patients in both datasets. Geometric mean LUAD/LUSC methylation, geometric mean ANT methylation, and univariate CpG AUCs between LUAD/LUSC and ANT were found for all patients. CpGs with lower mean methylation in LUAD/LUSC than ANT, hypermethylated in ANT (β>0.2), or with AUC between malignant tissue and ANT less than 0.8 were removed.

### LAMB Layer 3: Promoter CpG Methylation in NSCLC and Whole Blood

450K data of whole blood from the HCC LAMB Layer 3 analysis was utilized. Methylation frequency data for CpGs identified in NSCLC LAMB Layer 2 was extracted from the blood data. To perform a differential methylation analysis of LUSC/LUAD tissue to blood, 450K data for 287 separate TCGA LUADs and 237 separate LUSCs was downloaded. These tumors were separately matched whole blood samples from the healthy control patients based on patient age, gender, and race. The ratio of matched blood samples by GEO dataset mirrored the ratio of total blood samples by GEO dataset. CpGs with an AUC<0.8 between LUSC/LUAD tissue and blood were removed. Geometric mean methylation frequencies of remaining CpGs in the remaining blood samples were calculated. CpGs with a methylation frequency less than 0.1 in these unpaired blood samples were identified as LAMB-LUAD CpGs and LAMB-LUSC CpGs (depending on tissue type used).

### Tumor Tissue Analysis

450K methylation frequency data for 375 HCC, 502 PRAD, 409 CRC, 395 stomach adenocarcinoma (STAD), 31 cholangiocarcinoma (CHOL), 471 lung adenocarcinoma (LUAD), 185 esophageal carcinoma (ESCA), 183 pancreatic adenocarcinoma (PAAD), 513 low grade glioma (LGG), 140 glioblastoma multiforme (GBM), 793 breast invasive carcinoma (BRCA), 527 head-neck squamous cell carcinoma (HNSC), 418 urothelial bladder carcinoma (BLCA), 305 cervical squamous cell carcinoma and endocervical adenocarcinoma (CESC), 48 diffuse large B-cell lymphoma (DLBC), 324 kidney renal clear cell carcinoma (KIRC), 87 mesothelioma (MESO), 103 skin cutaneous melanoma (SKCM), 370 lung squamous cell carcinoma (LUSC), 436 uterine corpus endometrial carcinoma (ENDO), 80 adrenocortical carcinoma (ACC), 66 kidney chromophobe (KICH), 275 kidney renal papillary cell carcinoma (KIRP), 261 sarcoma (SARC), 504 thyroid carcinoma (THCA), 179 pheochromocytoma and paraganglioma (PCPG), 57 uterine carcinosarcoma (UCS), 80 uveal melanoma (UVM), 150 testicular germ cell (TGCT), 194 acute myeloid leukemia (AML), 124 thymoma (THYM), and 10 ovarian serous cystadenocarcinoma (OV) tumors was downloaded from TCGA. Methylation data for CpG sites in the LAMB-HCC, LAMB-CRC and LAMB-PRAD panels were extracted. 10-gene LAMB-HCC, 17-gene LAMB-CRC, and 5-gene LAMB-PRAD methylation frequency profiles were found for each tumor, as well as combined LAMB-HCC, LAMB-CRC and LAMB-PRAD scores. Methylation frequency profiles and combined panel scores were compared across cancer types. A Wilcoxon test between the primary cancer type (HCC, CRC, or PRAD) and other cancer types was used to determine statistical significance. Primary and secondary Gleason grades were added to find Gleason scores for PRAD. HCCs/CRCs and PRADs were separated by tumor stage and Gleason score, respectively; LAMB-HCC, LAMB-CRC, and LAMB-PRAD scores were compared across respective categories for respective cancer type. Wilcoxon tests were used to calculate statistical significance among the different tissue groups. For assessing LAMB's tissue classification ability, β_{LAMB-HCC}, β_{LAMB-CRC}, β_{LAMB-PRAD} scores for HCC, CRC, and PRAD were compared to predicting cancer type through the highest methylation score of the three LAMB panels.

### REFERENCES

Forshew, T., et al., Noninvasive identification and monitoring of cancer mutations by targeted deep sequencing of plasma DNA. Sci. Transl. Med. 4, 136ra68 (2012).
Dawson, S. J., et al., Analysis of circulating tumor DNA to monitor metastatic breast cancer. N. Engl. J. Med. 368, 1199-1209 (2013).
Bettegowda, C., et al. Detection of circulating tumor DNA in early- and late-stage human malignancies. Sci. Transl. Med. 6, 224ra24 (2014).
Cohen, J. D., et al. Detection and localization of surgically resectable cancers with a multi-analyte blood test. Science. 359, 926-930 (2018).
Corcoran, R. B. & Chabner, B. A. Application of cell-free DNA analysis to cancer treatment. N. Engl. J. Med. 379, 1754-1765 (2018).
McDonald, B. R., et al. Personalized circulating tumor DNA analysis to detect residual disease after neoadjuvant therapy in breast cancer. Sci. Transl. Med. 11, eaax7392 (2019).
Sun, K., et al., Plasma DNA tissue mapping by genome-wide methylation sequencing for noninvasive prenatal, cancer, and transplantation assessments. Proc. Natl. Acad. Sci. U.S.A 112, E5503-E5512 (2015).
Lamb, Y. N. & Dhillon, S. Epi proColon® 2.0 CE: A blood-based screening test for colorectal cancer. Mol. Diagn. Ther. 21, 225-232 (2017).
Xu, R., et al. Circulating tumour DNA methylation markers for diagnosis and prognosis of hepatocellular carcinoma. Nat. Mater. 16, 1155-1161 (2017).
Liu, L., et al. Targeted methylation sequencing of plasma cell-free DNA for cancer detection and classification. Ann. Oncol. 29, 1445-1453 (2018).
Liu, M. C., et al. Sensitive and specific multi-cancer detection and localization using methylation signatures in cell-free DNA. Ann. Oncol. 31, 745-759 (2020).
Luo, H., et al. Circulating tumor DNA methylation profiles enable early diagnosis, prognosis prediction, and screening for colorectal cancer. Sci. Transl. Med. 12, eaax7533 (2020).
Esteller, M. CpG island hypermethylation and tumor suppressor genes: a booming present, a brighter future. Oncogene 21, 5427-5440 (2002).
Jones, P. A. & Baylin, S. B. The fundamental role of epigenetic events in cancer. Nat. Rev. Genet. 3, 415-428 (2002).
Snyder, M. W., Kircher, M., Hill, A. J., Daza, R. M., & Shendure, J. Cell-free DNA comprises an in vivo nucleosome footprint that informs its tissues-of-origin. Cell 164, 57-68 (2016).
Mouliere, F., et al. Enhanced detection of circulating tumor DNA by fragment size analysis. Sci. Transl. Med. 10, eaat4921 (2018).
Chodavarapu, R. K. Relationship between nucleosome positioning and DNA methylation. Nature 466, 388-392 (2010).
Deng, J., et al. Targeted bisulfite sequencing reveals changes in DNA methylation associated with nuclear reprogramming. Nat. Biotechnol. 27, 353-360 (2009).
Sandoval, J., et al. Validation of a DNA methylation microarray for 450,000 CpG sites in the human genome. Epigenetics 6, 692-702 (2011)
Nielson, C. M. Testing complex genetic interactions for complex diseases, Sci. Transl. Med. 3, 87ec90 (2011).
Badve, S. Predictive biomarkers in oncology: applications in precision medicine (Springer Nature, Switzerland, A G, 2019), pp. 156.
Barbour, D. L. Precision medicine and the cursed dimensions. npj Digit. Med. 2, 4 (2019).
Subramanian, A., et al. Gene set enrichment analysis: A knowledge-based approach for interpreting genome-wide expression profiles. Proc. Natl. Acad. Sci. 102, 15545-15550 (2005).
Kim, D., Li, R., Lucas, A., Verma, S. S., Dudek, S. M., & Ritchie, M. D. Using knowledge-driven genomic interactions for multi-omics data analysis: metadimensional models for predicting clinical outcomes in ovarian carcinoma. J. Am. Med. Inform. Assoc. 24, 577-587 (2017).
Dahary, D., et al. Genome analysis and knowledge-driven variant interpretation with TGex. BMC Med. Genomics 12, 200 (2019).
C. G. A. Network. Comprehensive and integrative genomic characterization of hepatocellular carcinoma. Cell 169, 1327-1341 (2017).
C. G. A. Network. Comprehensive molecular characterization of human colon and rectal cancer. Nature 487, 330-337 (2012).
C. G. A. Network. The molecular taxonomy of primary prostate cancer. Cell 163, 1011-1025 (2015).
Zhang, D. Y. & Friedman, S. L. Fibrosis-dependent mechanisms of hepatocarcinogenesis. Hepatology 56, 769-775 (2012).
Yang, J. D., Hainaut, P., Gores, G. J., Amadou, A., Plymoth, A., & Roberts, L. R. A global view of hepatocellular carcinoma: trends, risk, prevention and management. Nat. Rev. Gastroenterol. Hepatol. 16, 589-604 (2019).
Singal, A. G., Pillai, A., & Tiro, J. Early detection, curative treatment, and survival rates for hepatocellular carcinoma surveillance in patients with cirrhosis: a meta-analysis. PLoS Med. 11, e1001624 (2014).
Johnson, P. J. The role of serum alpha-fetoprotein estimation in the diagnosis and management of hepatocellular carcinoma. Clin. Liver Dis. 5, 145-159 (2001).
Chang, T. S., et al. Alpha-fetoprotein measurement benefits hepatocellular carcinoma surveillance in patients with cirrhosis. Am. J. Gastroenterol. 110, 836-844 (2015).
Zhao, C., et al. Poor adherence to hepatocellular carcinoma surveillance: A systematic review and meta-analysis of a complex issue. Liver Int. 38, 503-514 (2018).
Hlady, R. A., et al., Genome-wide discovery and validation of diagnostic DNA methylation-based biomarkers for hepatocellular cancer detection in circulating cell free DNA. Theranostics 9, 7239-7250 (2019).
Marrero, J. A., et al. Alpha-fetoprotein, des-gamma carboxyprothrombin, and lectin-bound alpha-fetoprotein in early hepatocellular carcinoma. Gastroenterology 137, 110-118 (2009).
Wolf, A., et al. Colorectal cancer screening for average-risk adults: 2018 guideline update from the American Cancer Society. CA: a cancer journal for clinicians 68, 250-281 (2018).
Inadomi J. M., et al. Adherence to colorectal cancer screening: a randomized clinical trial of competing strategies. Arch Intern Med. 172, 575-582 (2012).
Goel, A., et al. EpiPanGI Dx: A cell-free DNA methylation fingerprint for the early detection of gastrointestinal cancers. NCBI GEO database. GSE149438, (2020).
López Torrecilla, J., et al. Uroncor consensus statement: Management of biochemical recurrence after radical radiotherapy for prostate cancer: From biochemical failure to castration resistance. Reports Pract. Oncol. Radiother. 20, 259-272 (2015).
Cornford, P., et al. EAU-ESTRO-SIOG Guidelines on Prostate Cancer. Part II: Treatment of Relapsing, Metastatic, and Castration-Resistant Prostate Cancer. Eur. Urol. 71, 630-642 (2017).
Mizokami, A., et al. Understanding prostate-specific antigen dynamics in monitoring metastatic castration-resistant prostate cancer: implications for clinical practice. Asian J. Androl. 19, 143-148 (2017).
Komura, K., Sweeney, C. J., Inamoto, T., Ibuki, N., Azuma, H., & Kantoff, P. W. Current treatment strategies for advanced prostate cancer. Int. J. Urol. 25, 220-231 (2018).
Gómez-Caamaño, A., et al. U. T. W. G. (URONCOR) Expert Panel for the Consensus on Management of Castration-Resistant Prostate Cancer 2017 Spanish Society of Radiation Oncology (SEOR), Consensus on management of castration-resistant prostate cancer on behalf of the Urological Tumours Working Group (URONCOR) of the Spanish Society of Radiation Oncology. Clin. Transl. Oncol. 21, 420-432 (2019).
Gordevidius, J., et al. Cell-free DNA modification dynamics in abiraterone acetate-treated prostate cancer patients. Clin. Cancer Res. 24, 3317-3324 (2018).
Rescigno, P., et al. Prostate-specific antigen decline After 4 weeks of treatment with abiraterone acetate and overall survival in patients with metastatic castration-resistant prostate cancer. Eur. Urol. 70, 724-731 (2016).
Nakayama, M., Kobayashi, H., Takahara, T., Oyama, R., Imanaka, K., & Yoshizawa, K. Association of early PSA decline and time to PSA progression in abiraterone acetate-treated metastatic castration-resistant prostate cancer; a post-hoc analysis of Japanese phase 2 trials. BMC Urol. 16, 27 (2016).
A. Fuerea, G. Baciarello, A. Patrikidou, L. Albigès, C. Massard, M. Di Palma, B. Escudier, K. Fizazi, Y. Loriot, Early PSA response is an independent prognostic factor in patients with metastatic castration-resistant prostate cancer treated with next-generation androgen pathway inhibitors, Eur. J. Cancer 61, 44-51 (2016).
K. Brasso, F. B. Thomsen, A. J. Schrader, S. C. Schmid, D. Lorente, M. Retz, A. S. Merseburger, C. A. von Klot, M. Boegemann, J. de Bono, Enzalutamide Antitumour Activity Against Metastatic Castration-resistant Prostate Cancer Previously Treated with Docetaxel and Abiraterone: A Multicentre Analysis, Eur. Urol. 68, 317-324 (2015).
Blum, A. et al. SnapShot: TCGA-Analyzed Tumors. Cell 173, 530 (2018).
Chang, H., et al. Methylation of tumor associated genes in tissue and plasma samples from liver disease patients. Exp. Mol. Pathol. 85, 96-100 (2008).
lizuka, N., et al. Efficient detection of hepatocellular carcinoma by a hybrid blood test of epigenetic and classical protein markers. Clin. Chim. Acta 412, 152-158 (2011).
Huang, W., et al. Analysis of DNA methylation in plasma for monitoring hepatocarcinogenesis. Genet. Test. Mol. Biomarkers 19, 295-302 (2015).
Lu, C. Y., et al. Cell-free methylation markers with diagnostic and prognostic potential in hepatocellular carcinoma. Oncotarget 8, 6406-6418 (2017).
Dong, X., et al. Diagnostic value of the methylation of multiple gene promoters in serum in hepatitis B virus-related hepatocellular carcinoma. Dis. Markers 2017, U.S. Pat. No. 2,929,381 (2017).
Constancio, V., et al. Early detection of the major male cancer types in blood-based liquid biopsies using a DNA methylation panel. Clin. Epigenetics 11, 175 (2019).
Brait, M., et al. Promoter methylation of MCAM, ERα and ERβ in serum of early stage prostate cancer patients. Oncotarget 8, 15431-15440 (2017).
Szklarczyk, D., et al. STRING v11: protein-protein association networks with increased coverage, supporting functional discovery in genome-wide experimental datasets. Nucleic Acids Res. 47, 607-613 (2019).
Reimand, J. Kull, M. Peterson, H. Hansen, J. & Vilo, J. g:Profiler—a web-based toolset for functional profiling of gene lists from large-scale experiments. Nucleic Acids Res. 35, 193-200 (2007).
Sidik, K. & Jonkman, J. N. A simple confidence interval for meta-analysis. Stat. Med. 21, 3153-3159 (2002).
Viechtbauer, W. Conducting meta-analyses in R with the metafor package, J. Stat. Software 36, (2010).
Leek, J. T., et al. The sva package for removing batch effects and other unwanted variation in high-throughput experiments. Bioinformatics 28, 882-883 (2012).
Robin, X., et al. pROC: an open-source package for R and S+ to analyze and compare ROC curves. BMC Bioinformatics 12, 77 (2011).
Kisiel, et al. Hepatocellular carcinoma detection by plasma methylated DNA: discovery, phase I pilot, and phase II clinical validation. Hepatology 69, 1180-1192 (2019).
Oussalah, A., et al. Plasma mSEPT9: a novel circulating cell-free DNA-based epigenetic biomarker to diagnose hepatocellular carcinoma. EBioMedicine 30, 138-147 (2018).

TABLE 1
LAMB-HCC panel with gene biomarker
functions and performance.

| Gene | n_{CpGs} | Function | AUC_{cfDNA} |
|---|---|---|---|
| AK055957 | 2 | Unknown | 0.71 |
| APC | 4 | Promotes β-Catenin Degradation | 0.77 |
| GSTP1 | 1 | Detoxifies Glutathione | 0.72 |
| HOXA1 | 3 | Transcription Factor | 0.77 |
| PFKP | 2 | Regulates Glycolysis | 0.68 |
| PRDM2 | 2 | Transcription Factor | 0.77 |
| RUNX3 | 2 | Transcription Factor | 0.65 |
| SEPTIN9 | 2 | Cytokinesis, Cell Cycle Control | 0.71 |
| SPINT2 | 1 | Inhibits Hepatocyte Growth Factor | 0.65 |
| WIF1 | 2 | Inhibits Wnt Protein | 0.64 |

TABLE 2
LAMB-CRC panel with gene biomarker
functions and performance.

| Gene | n_{CpGs} | Function | AUC_{cfDNA} |
|---|---|---|---|
| ALX4 | 1 | Transcription Factor | NA |
| CNRIP1 | 1 | Cannabinoid Receptor C-Terminal Tail | NA |
| COL4A1 | 1 | Collagen Alpha Protein | 0.84 |
| COL4A2 | 1 | Collagen Alpha Protein | 0.64 |
| EFEMP1 | 2 | Extracellular Glycoprotein | 0.82 |
| EYA4 | 9 | DNA Repair Complex | 0.79 |
| FBN1 | 2 | Extracellular Glycoprotein | 0.72 |
| HIC1 | 1 | Growth Regulator | NA |
| IKZF1 | 6 | Transcription Factor | 0.68 |
| MIR34C | 2 | Post-Transcriptional Regulator | 0.70 |
| NDRG4 | 1 | Cell Cycle Progression | 0.71 |
| SDC2 | 4 | Transmembrane Proteoglycan | 0.56 |
| SEPTIN9 | 4 | Cytokinesis, Cell Cycle Control | 0.75 |
| SOX21 | 4 | DNA Binding Protein | 0.85 |
| TFPI2 | 1 | Serine Protease Inhibitor | NA |
| TMEFF2 | 1 | Transmembrane Protein | NA |
| UNC5C | 5 | Netrin Receptor | 0.81 |

TABLE 3
LAMB-PRAD panel with gene biomarker
functions and performance.

| Gene | n_{CpGs} | Function | AUC_{cfDNA} |
|---|---|---|---|
| APC | 8 | Promotes β-Catenin Degradation | 0.81 |
| CD44 | 1 | Cell Migration and Adhesion | 0.79 |
| PYCARD | 3 | Activates Caspase (Apoptosis) | 0.89 |
| RARB | 6 | Inhibits Cell Growth (Retinoic Acid) | 0.89 |
| RBP1 | 1 | Transports Retinol | 0.79 |

TABLE 4
Datasets used in the LAMB-HCC discovery process.

| Layer | Dataset | Type (n) | Description |
|---|---|---|---|
| 1: HCC/ANT Meta- | 67 published | HCC paired to | Inclusion criteria: English; |
| Analysis | tissue papers | ANT (3425) | paired HCC/ANT samples; |
| | | | used MSP or QMSP; |
| | | | genes tested in other |
| | | | papers |
| | 2 published | HCC, non- | Inclusion Criteria: |
| | cfDNA | HCC cirrhosis | AUC >0.8 (HCC vs. non-HCC |
| | papers | (435) | patients) |
| 2: HCC/ANT | TCGA-LIHC | HCC/ANT | Paired primary HCC/ANT |
| Microarray | | (50) | |
| | GSE 54503 | HCC/ANT | Paired primary HCC/ANT |
| | | (66) | |
| | GSE 89852 | HCC/ANT | Paired primary HCC/ANT |
| | | (37) | |
| 3: HCC/Blood | TCGA-LIHC | HCC (159) | Not analyzed in Layer 2; |
| Microarray | | | matched to healthy controls |
| | GSE 84727 | Blood (305) | Healthy control lysed blood |
| | GSE 80417 | Blood (127) | Healthy control lysed blood |
| | GSE 40279 | Blood (622) | Healthy control lysed blood |
| | GSE 72773 | Blood (272) | Healthy control lysed blood |
| | GSE 111629 | Blood (236) | Healthy control lysed blood |
| | GSE 53740 | Blood (160) | Healthy control lysed blood |

HCC: Hepatocellular Carcinoma; ANT: Adjacent Noncancerous Tissue; MSP: Methylation-Specific Polymerase chain reaction; QMSP: Quantitative MSP; cfDNA: cell-free DNA; AUC: Area Under Curve.

TABLE 5
Datasets used in the LAMB-CRC discovery process.

| Layer | Dataset | Type (n) | Description |
|---|---|---|---|
| 1: CRC/ANT Meta- | 188 published | CRC paired to | Inclusion criteria: English; |
| Analysis | tissue papers | ANT (12667) | control samples are ANT |
| | | | from same CRC patients; |
| | | | used MSP or qMSP; genes |
| | | | tested in other paper |
| 2: CRC/ANT | TCGA-COAD, | CRC/ANT (44) | Paired CRC/ANT |
| Microarray | TCGA-READ | | |
| | GSE 77718 | CRC/ANT (95) | Paired CRC/ANT |
| | GSE 101764 | CRC/ANT (103) | Paired CRC/ANT |
| 3: CRC/Blood | TCGA-COAD, | CRC (280) | Not analyzed in Layer 2; |
| Microarray | TCGA-READ | | matched to healthy controls |
| | GSE 84727 | Blood (305) | Healthy control lysed |
| | | | blood |
| | GSE 80417 | Blood (127) | Healthy control lysed |
| | | | blood |
| | GSE 40279 | Blood (622) | Healthy control lysed |
| | | | blood |
| | GSE 72773 | Blood (272) | Healthy control lysed |
| | | | blood |
| | GSE 111629 | Blood (236) | Healthy control lysed |
| | | | blood |
| | GSE 53740 | Blood (160) | Healthy control lysed |
| | | | blood |

CRC: Colorectal Adenocarcinoma; ANT: Adjacent Noncancerous Tissue; MSP: Methylation-Specific Polymerase chain reaction; QMSP: Quantitative MSP; cfDNA: cell-free DNA; AUC: Area Under Curve.

TABLE 6
Datasets used in the LAMB-PRAD discovery process.

| Layer | Dataset | Type (n) | Description |
|---|---|---|---|
| 1: PRAD/ANT Meta- | 41 published | PRAD paired to | Inclusion criteria: English; |
| Analysis | tissue papers | ANT (2663) | control samples are ANT |
| | | | from same PRAD patients; |
| | | | used MSP or qMSP; genes |
| | | | tested in other paper |
| 2: PRAD/ANT | TCGA-PRAD | PRAD/ANT (50) | Paired PRAD/ANT |
| Microarray | GSE 112047 | PRAD/ANT (15) | Paired PRAD/ANT |
| | GSE 55598 | PRAD/ANT (16) | Paired PRAD/ANT |
| | GSE 73549 | PRAD/ANT (12) | Paired PRAD/ANT |
| | GSE 76938 | PRAD/ANT (52) | Paired PRAD/ANT |
| 3: PRAD/Blood | TCGA-PRAD | PRAD (315) | Not analyzed in Layer 2; |
| Microarray | | | matched to healthy controls |
| | GSE 84727 | Blood (305) | Healthy control lysed |
| | | | blood |
| | GSE 80417 | Blood (127) | Healthy control lysed |
| | | | blood |
| | GSE 40279 | Blood (622) | Healthy control lysed |
| | | | blood |
| | GSE 72773 | Blood (272) | Healthy control lysed |
| | | | blood |
| | GSE 111629 | Blood (236) | Healthy control lysed |
| | | | blood |
| | GSE 53740 | Blood (160) | Healthy control lysed |
| | | | blood |

PRAD: Prostate Adenocarcinoma; ANT: Adjacent Noncancerous Tissue; MSP: Methylation-Specific Polymerase chain reaction; QMSP: Quantitative MSP; cfDNA: cell-free DNA; AUC: Area Under Curve.

TABLE 7
Diagnostic odds ratios of hepatocellular
carcinoma genes analyzed by meta-analysis

| | | |
|---|---|---|
| Gene | DOR (95% CI) | n |

| | | | |
|---|---|---|---|
| PRDM2 | 14.4 | (8.1, 25.4) | 452 |
| RUNX3 | 21.3 | (7.1, 63.5) | 420 |
| RASSF1A | 9.7 | (17.3, 5.4) | 1077 |
| CDKN2A (p16) | 6.0 | (8.7, 4.1) | 1330 |
| TFPI2 | 10.1 | (26.8, 3.8) | 272 |
| GSTP1 | 6.3 | (10.6, 3.7) | 677 |
| SOCS1 | 8.4 | (21.2, 3.4) | 508 |
| SPINT2 | 21.8 | (170.6, 2.8) | 125 |
| DAB2IP | 9.5 | (33.7, 2.7) | 201 |
| APC | 7.1 | (23.3, 2.2) | 470 |
| SFRP1 | 7.9 | (36.1, 1.7) | 229 |
| TIMP3 | 3.6 | (9.2, 1.4) | 149 |
| WT1 | 3.6 | (9.8, 1.3) | 138 |
| WIF1 | 4.7 | (20.7, 1.1) | 198 |
| CDKN2A (p14) | 1.8 | (3.2, 1.0) | 529 |
| MLH1 | 3.1 | (9.2, 1.0) | 258 |
| TP73 | 4.5 | (23.0, 0.88) | 136 |
| SFRP2 | 2.9 | (9.3, 0.9) | 269 |
| EDNRB | 5.6 | (37.7, 0.8) | 142 |
| PTGS2 | 8.6 | (94.6, 0.8) | 285 |
| PTEN | 2.2 | (6.3, 0.7) | 133 |
| RB1 | 1.5 | (3.2, 0.7) | 210 |
| SOCS3 | 3.9 | (21.9, 0.7) | 138 |
| TP53 | 3.4 | (19.1, 0.6) | 107 |
| RARB | 2.2 | (8.0, 0.6) | 137 |
| CDH1 | 1.3 | (2.7, 0.6) | 546 |
| CDKN2B | 1.2 | (3.3, 0.5) | 329 |
| MGMT | 1.0 | (2.6, 0.4) | 421 |
| CAV1 | 8.1 | (213.4, 0.3) | 104 |
| STK11 | 2.1 | (17.3, 0.3) | 136 |
| DAPK | 1.0 | (5.4, 0.2) | 215 |
| HIC1 | 2.0 | (27.6, 0.1) | 252 |
| RASSF1C | 1.0 | (9.7, 0.1) | 169 |

After calculating diagnostic odds ratios for each gene in each HCC paper, the Hartung-Knapp-Sidik-Jonkman estimator was used to find adjusted DORs. Genes are ordered by lower limit of the 95% confidence interval of DOR. HCC: Hepatocellular Carcinoma, DOR: Diagnostic Odds Ratio.

TABLE 8
Diagnostic odds ratios of colorectal adenocarcinoma
genes analyzed by meta-analysis

| | | |
|---|---|---|
| Gene | DOR (95% CI) | n |

| | | | |
|---|---|---|---|
| SPG20 | 229.5 | (55.2, 953.6) | 317 |
| SNCA | 186.5 | (42.9, 811.2) | 194 |
| NPY | 134.2 | (4.8, 3730.7) | 82 |
| SOX7 | 133.1 | (13.2, 1338.7) | 131 |
| PCDH10 | 114 | (37, 351.2) | 147 |
| FBN1 | 94.2 | (45.4, 195.6) | 269 |
| COL4A2 | 75.6 | (29, 197.2) | 115 |
| MAL | 63.4 | (15.9, 253.1) | 216 |
| MIR34B.C | 61.4 | (17.2, 219) | 342 |
| COL4A1 | 59.2 | (24.3, 144) | 173 |
| SEPTIN9 | 53.9 | (26.5, 109.5) | 364 |
| TMEFF2 | 53.1 | (27.2, 103.5) | 330 |
| NDRG2 | 52.3 | (10.9, 249.7) | 57 |
| CNRIP1 | 51.4 | (4.7, 558) | 210 |
| ITGA4 | 48.1 | (10.9, 211.1) | 206 |
| SDC2 | 41.1 | (4.4, 385.3) | 191 |
| CDH4 | 39.7 | (6.3, 250.8) | 258 |
| UNC5C | 33.2 | (8.3, 132.6) | 151 |
| RASSF2 | 32.6 | (4.4, 241.7) | 176 |
| IKZF1 | 31 | (13.6, 70.7) | 246 |
| NKX6.1 | 30.6 | (9.5, 98.3) | 302 |
| BCAT1 | 26.7 | (4, 178.6) | 157 |
| DKK3 | 25.4 | (3.8, 169.2) | 258 |
| HIC1 | 25.2 | (3.3, 192.4) | 155 |
| WNT5A | 24.9 | (3.3, 187.4) | 207 |
| EYA4 | 24 | (10.7, 53.7) | 106 |
| GATA5 | 22 | (5.4, 89.9) | 166 |
| VIM | 20.4 | (10.5, 39.6) | 218 |
| WIF1 | 20.2 | (6.7, 60.8) | 295 |
| SFRP4 | 17.8 | (5, 62.8) | 393 |
| ALX4 | 17.5 | (3.3, 92.3) | 174 |
| SFRP1 | 17 | (7.7, 37.4) | 742 |
| FOXI2 | 16.7 | (1.9, 144) | 124 |
| OSMR | 16.6 | (3.7, 75.2) | 367 |
| DKK1 | 15.9 | (2.7, 93.8) | 105 |
| SOX21 | 15.3 | (3.3, 72) | 108 |
| ADAMTS1 | 15 | (4.5, 50.8) | 113 |
| KL | 15 | (6.3, 35.5) | 274 |
| NR3C1 | 14.5 | (1.9, 110.6) | 101 |
| GATA4 | 11.8 | (3.8, 36.8) | 151 |
| SFRP2 | 11.7 | (2.6, 53.4) | 621 |
| APBA2 | 9.9 | (3.3, 30) | 185 |
| UCHL1 | 9.4 | (3, 29) | 48 |
| APBA1 | 9.2 | (3.1, 26.9) | 251 |
| CDX2 | 8.6 | (0.7, 110.3) | 213 |
| DLEC1 | 8.6 | (4.3, 17) | 190 |
| NDRG4 | 8.5 | (1.4, 51.7) | 334 |
| ID4 | 8.2 | (0.8, 81.5) | 196 |
| MINT31 | 8.1 | (2.6, 25.3) | 272 |
| DKK2 | 7.9 | (1, 64.5) | 35 |
| SFRP5 | 7.7 | (2.8, 21) | 430 |
| RARB | 7.6 | (1.8, 32) | 510 |
| TWIST1 | 7.5 | (0.8, 71.5) | 492 |
| RUNX3 | 7.4 | (3.4, 16.4) | 338 |
| CDH13 | 7.3 | (2.2, 24.8) | 362 |
| QKI | 7.2 | (1.2, 41.6) | 20 |
| BMP3 | 6.9 | (1.1, 42.6) | 227 |
| CRABP1 | 6.9 | (2.2, 21.7) | 150 |
| SOCS1 | 6.9 | (3.4, 14.1) | 555 |
| SEZ6L | 6.8 | (2.2, 21.1) | 57 |
| SCGB3A1 | 6.5 | (1.6, 27) | 86 |
| CDKN2A (p14) | 6.4 | (2.2, 18.5) | 808 |
| HLTF | 6.4 | (1.6, 25.2) | 321 |
| CHFR | 6 | (1.7, 22.1) | 301 |
| CDKN2A (p16) | 5.8 | (3.3, 10.2) | 2412 |
| MGMT | 5.5 | (3.5, 8.6) | 2376 |
| APC | 5.4 | (2.4, 12.4) | 1633 |
| CCNA1 | 5.3 | (0.4, 69.4) | 89 |
| ATM | 4.9 | (0.7, 36) | 125 |
| EFEMP1 | 4.8 | (2.8, 8.4) | 180 |
| TFPI2 | 4.6 | (1.9, 11.1) | 124 |
| IGF2 | 4.3 | (1.1, 16.9) | 131 |
| GSTP1 | 4.2 | (1.5, 12.1) | 68 |
| PYCARD | 4.1 | (1.6, 10.5) | 62 |
| CDKN2A | 3.6 | (2, 6.6) | 561 |
| NEUROG1 | 3.6 | (1.2, 10.3) | 131 |
| MLH1 | 3.5 | (2.1, 5.7) | 2654 |
| MSH2 | 3.4 | (0.7, 16.6) | 210 |
| RASSF1 | 3.3 | (1.8, 5.9) | 905 |
| TIMP3 | 3.2 | (1, 10.9) | 414 |
| BRCA1 | 3.1 | (0.6, 16) | 169 |
| CACNA1G | 2.8 | (1.4, 5.5) | 235 |
| CADM1 | 2.7 | (0.6, 12.6) | 158 |
| TAC1 | 2.7 | (0.5, 14.4) | 44 |
| RTL8C | 2.3 | (0.6, 8.4) | 72 |
| COX2 | 2.2 | (1.2, 4.3) | 220 |
| CDKN2B | 2.2 | (1, 4.8) | 545 |
| CDH1 | 2.2 | (0.9, 5) | 655 |
| DAPK1 | 2.2 | (0.9, 5.3) | 397 |
| IGFBP7 | 1.3 | (0.1, 12.5) | 375 |
| TIMP2 | 0.5 | (0, 7.6) | 9 |
| SNCG | 0.2 | (0, 0.9) | 80 |

After calculating diagnostic odds ratios for each gene in each CRC paper, the Hartung-Knapp-Sidik-Jonkman estimator was used to find adjusted DORs. Genes are ordered by the lower limit of the 95% confidence interval of their DOR. CRC: Colorectal Adenocarcinoma, DOR: Diagnostic Odds Ratio.

TABLE 9
Diagnostic odds ratios of prostate adenocarcinoma
genes analyzed by meta-analysis

| | | |
|---|---|---|
| Gene | DOR (95% CI) | n |

| | | | |
|---|---|---|---|
| GSTP1 | 21.3 | (10.8, 41.9) | 1171 |
| MIR34A | 48.7 | (10.4, 227.7) | 49 |
| RARB | 14.3 | (6.2, 32.8) | 593 |
| APC | 9.3 | (4.3, 20.2) | 706 |
| RASSF1 | 12.6 | (3.9, 40.3) | 746 |
| CD44 | 24.6 | (2.5, 240.7) | 16 |
| PYCARD | 3.7 | (2.0, 6.9) | 177 |
| ESR2 | 34.6 | (1.1, 1075.4) | 88 |
| RBP1 | 6.5 | (1.0, 41.9) | 154 |
| HIC1 | 6.1 | (0.8, 43.7) | 191 |
| CCND2 | 4.9 | (0.8, 31.6) | 259 |
| TNFRSF10D | 3.6 | (0.6, 20.6) | 66 |
| EDNRB | 5.1 | (0.6, 42.4) | 132 |
| ESR1 | 80.3 | (0.6, 10723.7) | 111 |
| ABCB1 | 18.0 | (0.5, 685.0) | 194 |
| AR | 2.1 | (0.4, 10.5) | 147 |
| CDKN2A (p16) | 0.7 | (0.3, 1.6) | 268 |
| DAPK1 | 2.7 | (0.3, 22.6) | 219 |
| PTGS2 | 3.9 | (0.3, 56.0) | 146 |
| TIMP3 | 1.1 | (0.2, 4.6) | 300 |
| CDKN2A (p14) | 0.8 | (0.2, 2.5) | 259 |
| MGMT | 0.6 | (0.2, 1.8) | 367 |
| SFN | 22.9 | (0.1, 7479.0) | 162 |
| CDH1 | 1.4 | (0.0, 51.3) | 110 |
| ID4 | 8.5 | (0.0, 182.4) | 134 |

After calculating diagnostic odds ratios for each gene in each PRAD paper, the Hartung-Knapp-Sidik-Jonkman estimator was used to find adjusted DORs. Genes are ordered by the lower limit of the 95% confidence interval of their DOR. PRAD: Prostate Adenocarcinoma, DOR: Diagnostic Odds Ratio.

TABLE 10
Methylation frequency and AUC values for LAMB-HCC CpGs.

| CpG | Gene | β_{HCC, Mean} | β_{ANT, Mean} | Δβ_{Mean} | AUC_{HCC/ANT} | AUC_{HCC/Blood} | β_{Blood, Mean} | AUC_{cfDNA} |
|---|---|---|---|---|---|---|---|---|
| cg08572734 | AK055957 | 0.2768 | 0.1058 | 0.1710 | 0.9075 | 0.9408 | 0.0556 | 0.7562 |
| cg15607538 | AK055957 | 0.4180 | 0.1165 | 0.3015 | 0.9562 | 0.9341 | 0.0763 | 0.6921 |
| cg08571859 | APC | 0.4445 | 0.1297 | 0.3148 | 0.9288 | 0.8673 | 0.0625 | 0.8347 |
| cg14479889 | APC | 0.4913 | 0.1808 | 0.3104 | 0.9219 | 0.8985 | 0.0538 | 0.7479 |
| cg03667968 | APC | 0.4812 | 0.1078 | 0.3734 | 0.9286 | 0.9803 | 0.0130 | 0.7417 |
| cg00577935 | APC | 0.4306 | 0.1121 | 0.3185 | 0.9224 | 0.8726 | 0.0459 | 0.7211 |
| cg02659086 | GSTP1 | 0.2151 | 0.0350 | 0.1801 | 0.8958 | 0.9023 | 0.0218 | 0.7190 |
| cg26744375 | HOXA1 | 0.2119 | 0.0861 | 0.1258 | 0.8105 | 0.8773 | 0.0377 | 0.7438 |
| cg09420439 | HOXA1 | 0.3135 | 0.1288 | 0.1847 | 0.8703 | 0.9331 | 0.0436 | 0.6818 |
| cg04673590 | HOXA1 | 0.2108 | 0.0694 | 0.1414 | 0.8838 | 0.8464 | 0.0425 | 0.6384 |
| cg08465862 | PFKP | 0.2650 | 0.0982 | 0.1668 | 0.8089 | 0.8510 | 0.0533 | 0.7417 |
| cg14250130 | PFKP | 0.2518 | 0.0331 | 0.2188 | 0.9481 | 0.8616 | 0.0351 | 0.6405 |
| cg00922376 | PRDM2 | 0.3131 | 0.0386 | 0.2745 | 0.9010 | 0.9931 | 0.0067 | 0.7355 |
| cg05346841 | PRDM2 | 0.2841 | 0.0432 | 0.2409 | 0.8937 | 0.9817 | 0.0108 | 0.7149 |
| cg13629563 | RUNX3 | 0.2543 | 0.1162 | 0.1380 | 0.8099 | 0.8953 | 0.0592 | 0.6467 |
| cg26421310 | RUNX3 | 0.2984 | 0.1213 | 0.1771 | 0.8281 | 0.9330 | 0.0398 | 0.5620 |
| cg17300544 | SEPTIN9 | 0.2509 | 0.0419 | 0.2090 | 0.8072 | 0.8496 | 0.0227 | 0.7335 |
| cg06848185 | SEPTIN9 | 0.2439 | 0.0367 | 0.2071 | 0.8820 | 0.9280 | 0.0192 | 0.6219 |
| cg22522066 | SPINT2 | 0.3488 | 0.1679 | 0.1809 | 0.8538 | 0.9010 | 0.0915 | 0.6467 |
| cg26397188 | WIF1 | 0.3989 | 0.1795 | 0.2194 | 0.9084 | 0.9393 | 0.0876 | 0.6653 |
| cg24166864 | WIF1 | 0.3584 | 0.1739 | 0.1845 | 0.8416 | 0.8965 | 0.0842 | 0.5868 |

CpG: Illumina HumanMethylation450K Manifest identifier for CpG site; β_{HCC, Mean}: geometric mean methylation frequency of paired hepatocellular carcinomas (HCC); β_{ANT, Mean}: geometric mean methylation frequency of paired adjacent noncancerous tissues (ANT); Δβ_{Mean}: difference between β_{HCC, Mean} and β_{ANT, Mean}; AUC_{HCC/ANT}: area under curve (AUC) for ROC curve between paired HCCs and ANTs; AUC_{HCC/Blood}: area under curve (AUC) for ROC curve between separate paired HCCs and blood; β_{Blood, Mean}: geometric mean methylation frequency of remaining unpaired blood samples; AUC_{cfDNA}: area under curve (AUC) for ROC curve between HCC and cirrhosis cfDNA.

TABLE 11
Methylation frequency and AUC values for LAMB-CRC CpGs.

| CpG | Gene | β_{CRC, Mean} | β_{ANT, Mean} | Δβ_{Mean} | AUC_{CRC/ANT} | AUC_{CRC/Blood} | β_{Blood, Mean} |
|---|---|---|---|---|---|---|---|
| cg14410064 | ALX4 | 0.336129 | 0.195692 | 0.140437 | 0.803992 | 0.825077 | 0.085667 |
| cg10908369 | CNRIP1 | 0.325885 | 0.198569 | 0.127316 | 0.840192 | 0.973023 | 0.070603 |
| cg25530246 | COL4A1 | 0.27755 | 0.127654 | 0.149895 | 0.88049 | 0.89125 | 0.050046 |
| cg21938148 | COL4A2 | 0.560445 | 0.186154 | 0.374291 | 0.960436 | 0.986148 | 0.023944 |
| cg03817671 | EFEMP1 | 0.285118 | 0.136946 | 0.148172 | 0.823544 | 0.916773 | 0.018809 |
| cg25046074 | EFEMP1 | 0.295621 | 0.181747 | 0.113874 | 0.804095 | 0.929554 | 0.074932 |
| cg08712932 | EYA4 | 0.457506 | 0.188519 | 0.268987 | 0.939929 | 0.974643 | 0.06789 |
| cg11664500 | EYA4 | 0.518513 | 0.17893 | 0.339582 | 0.946725 | 0.976518 | 0.063982 |
| cg12152919 | EYA4 | 0.3718 | 0.181132 | 0.190667 | 0.905676 | 0.97611 | 0.064097 |
| cg14017655 | EYA4 | 0.372688 | 0.135052 | 0.237636 | 0.873694 | 0.927985 | 0.028105 |
| cg14287112 | EYA4 | 0.504491 | 0.163152 | 0.341338 | 0.951028 | 0.973903 | 0.059132 |
| cg16347317 | EYA4 | 0.297327 | 0.12332 | 0.174007 | 0.896387 | 0.943189 | 0.033123 |
| cg20330472 | EYA4 | 0.380655 | 0.165066 | 0.215589 | 0.896967 | 0.97185 | 0.04744 |
| cg20611276 | EYA4 | 0.396742 | 0.158863 | 0.237879 | 0.911823 | 0.971339 | 0.036235 |
| cg22871668 | EYA4 | 0.504087 | 0.152019 | 0.352069 | 0.952428 | 0.976441 | 0.046787 |
| cg10480343 | FBN1 | 0.38632 | 0.133278 | 0.253042 | 0.882078 | 0.914439 | 0.028463 |
| cg22535307 | FBN1 | 0.410839 | 0.149314 | 0.261525 | 0.889693 | 0.95764 | 0.017262 |
| cg25520679 | HIC1 | 0.294866 | 0.178176 | 0.11669 | 0.823544 | 0.899209 | 0.075864 |
| cg07589773 | IKZF1 | 0.449218 | 0.188713 | 0.260505 | 0.896831 | 0.856964 | 0.070343 |
| cg16697214 | IKZF1 | 0.441023 | 0.191036 | 0.249987 | 0.897019 | 0.969311 | 0.030317 |
| cg18607529 | IKZF1 | 0.315476 | 0.093383 | 0.222093 | 0.873438 | 0.919375 | 0.011869 |
| cg18810347 | IKZF1 | 0.307687 | 0.14995 | 0.157736 | 0.87687 | 0.883763 | 0.075029 |
| cg20078466 | IKZF1 | 0.348166 | 0.09805 | 0.250116 | 0.886995 | 0.959707 | 0.011015 |
| cg27633530 | IKZF1 | 0.372064 | 0.133035 | 0.239029 | 0.888618 | 0.884681 | 0.030343 |
| cg01192900 | MIR34C | 0.38343 | 0.191924 | 0.191506 | 0.902193 | 0.956964 | 0.094599 |
| cg24041078 | MIR34C | 0.355636 | 0.155939 | 0.199697 | 0.864183 | 0.932883 | 0.043583 |
| cg06650115 | NDRG4 | 0.308206 | 0.128916 | 0.17929 | 0.80266 | 0.876148 | 0.029296 |
| cg13096260 | SDC2 | 0.446243 | 0.178445 | 0.267799 | 0.885629 | 0.86551 | 0.076596 |
| cg18719750 | SDC2 | 0.405797 | 0.190154 | 0.215643 | 0.888652 | 0.904834 | 0.099437 |
| cg24732574 | SDC2 | 0.32863 | 0.146808 | 0.181822 | 0.88049 | 0.899082 | 0.066634 |
| cg25070637 | SDC2 | 0.359216 | 0.164331 | 0.194885 | 0.863619 | 0.851225 | 0.081893 |
| cg02884239 | SEPT9 | 0.289501 | 0.160342 | 0.129159 | 0.809251 | 0.849145 | 0.080061 |
| cg06848185 | SEPT9 | 0.30327 | 0.107967 | 0.195303 | 0.859658 | 0.944043 | 0.019305 |
| cg15044248 | SEPT9 | 0.293549 | 0.100441 | 0.193109 | 0.839458 | 0.848125 | 0.022133 |
| cg19554255 | SEPT9 | 0.305942 | 0.147625 | 0.158317 | 0.859931 | 0.902717 | 0.071482 |
| cg00800383 | SOX21 | 0.320898 | 0.171484 | 0.149414 | 0.815108 | 0.880319 | 0.080573 |
| cg19063972 | SOX21 | 0.353902 | 0.185286 | 0.168616 | 0.830254 | 0.88662 | 0.087869 |
| cg19184934 | SOX21 | 0.344477 | 0.188497 | 0.15598 | 0.83536 | 0.893941 | 0.09919 |
| cg27442308 | SOX21 | 0.493708 | 0.185013 | 0.308695 | 0.932194 | 0.979247 | 0.033476 |
| cg22441533 | TFPI2 | 0.283126 | 0.145915 | 0.137212 | 0.871542 | 0.993061 | 0.01649 |
| cg01808545 | TMEFF2 | 0.413412 | 0.186456 | 0.226956 | 0.875316 | 0.940663 | 0.064017 |
| cg01194057 | UNC5C | 0.341678 | 0.161086 | 0.180592 | 0.868742 | 0.923138 | 0.043107 |
| cg07234102 | UNC5C | 0.303981 | 0.177157 | 0.126825 | 0.846817 | 0.955421 | 0.067975 |
| cg12584684 | UNC5C | 0.44731 | 0.178321 | 0.268989 | 0.914828 | 0.933699 | 0.081857 |
| cg15701178 | UNC5C | 0.37122 | 0.152623 | 0.218597 | 0.915784 | 0.946518 | 0.049148 |
| cg21782409 | UNC5C | 0.491986 | 0.194493 | 0.297492 | 0.923656 | 0.941441 | 0.029269 |

CpG: Illumina HumanMethylation450K Manifest 450K identifier for CpG site; β_{CRC, Mean}: geometric mean methylation frequency of paired colorectal adenocarcinomas (CRC); β_{ANT, Mean}: geometric mean methylation frequency of paired adjacent noncancerous tissues (ANT); Δβ_{Mean}: difference between β_{CRC, Mean} and β_{ANT, Mean}; AUC_{CRC/ANT}: area under curve (AUC) for ROC curve between paired CRCs and ANTs; AUC_{CRC/Blood}: area under curve (AUC) for ROC curve between separate paired CRCs and blood; β_{Blood, Mean}: geometric mean methylation frequency of remaining unpaired blood samples.

TABLE 12
Methylation frequency and AUC values for LAMB-PRAD CpGs

| CpG | Gene | β_{PRAD, Mean} | β_{ANT, Mean} | Δβ_{Mean} | AUC_{PRAD/ANT} | AUC_{PRAD/Blood} | β_{Blood, Mean} | AUC_{cfDNA} |
|---|---|---|---|---|---|---|---|---|
| cg00577935 | APC | 0.1794 | 0.0386 | 0.1409 | 0.8387 | 0.9103 | 0.0459 | 0.8016 |
| cg08571859 | APC | 0.2068 | 0.0544 | 0.1525 | 0.8412 | 0.8871 | 0.0626 | 0.8413 |
| cg11613015 | APC | 0.1641 | 0.0370 | 0.1271 | 0.8264 | 0.9901 | 0.0134 | 0.8492 |
| cg14479889 | APC | 0.2504 | 0.0646 | 0.1858 | 0.8486 | 0.9232 | 0.0538 | 0.7698 |
| cg14511739 | APC | 0.1862 | 0.0425 | 0.1437 | 0.8300 | 0.9328 | 0.0247 | 0.6746 |
| cg16970232 | APC | 0.2682 | 0.0645 | 0.2037 | 0.8562 | 0.9171 | 0.0615 | 0.8016 |
| cg22035501 | APC | 0.1932 | 0.0468 | 0.1464 | 0.8316 | 0.9515 | 0.0280 | 0.7143 |
| cg23938220 | APC | 0.2742 | 0.0716 | 0.2026 | 0.8608 | 0.9489 | 0.0262 | 0.6349 |
| cg08530414 | CD44 | 0.2105 | 0.0826 | 0.1279 | 0.8049 | 0.8897 | 0.0802 | 0.7937 |
| cg05214748 | PYCARD | 0.4035 | 0.1947 | 0.2088 | 0.9098 | 0.9996 | 0.0985 | 0.8651 |
| cg05907835 | PYCARD | 0.3470 | 0.1609 | 0.1860 | 0.9003 | 0.9980 | 0.0801 | 0.8889 |
| cg15468095 | PYCARD | 0.1950 | 0.0573 | 0.1377 | 0.9284 | 0.9718 | 0.0717 | 0.7381 |
| cg02499249 | RARB | 0.3746 | 0.0755 | 0.2991 | 0.9179 | 0.9773 | 0.0629 | 0.7063 |
| cg06720425 | RARB | 0.2835 | 0.0783 | 0.2051 | 0.9153 | 0.9719 | 0.0803 | 0.8889 |
| cg12479047 | RARB | 0.2653 | 0.0470 | 0.2183 | 0.8999 | 0.9406 | 0.0372 | 0.7460 |
| cg18094781 | RARB | 0.3024 | 0.0767 | 0.2258 | 0.8932 | 0.9270 | 0.0791 | 0.7778 |
| cg20899354 | RARB | 0.2588 | 0.0436 | 0.2152 | 0.8977 | 0.9682 | 0.0199 | 0.8254 |
| cg26124016 | RARB | 0.2854 | 0.0509 | 0.2344 | 0.8938 | 0.9348 | 0.0431 | 0.8889 |
| cg15229124 | RBP1 | 0.4274 | 0.1209 | 0.3065 | 0.9154 | 0.9902 | 0.0534 | 0.7937 |

CpG: Illumina HumanMethylation450K Manifest 450K identifier for CpG site; β_{PRAD, Mean}: geometric mean methylation frequency of paired prostate adenocarcinomas (PRAD); β_{ANT, Mean}: geometric mean methylation frequency of paired adjacent noncancerous tissues (ANT); Δβ_{Mean}: difference between β_{PRAD, Mean} and β_{ANT, Mean}; AUC_{PRAD/ANT}: area under curve (AUC) for ROC curve between paired PRADs and ANTs; AUC_{PRAD/Blood}: area under curve (AUC) for ROC curve between paired PRADs and blood; β_{Blood, Mean}: geometric mean methylation frequency of remaining unpaired blood samples; AUC_{cfDNA}: area under curve (AUC) for ROC curve between responder and non-responder cfDNA.

TABLE 13
Methylation frequency and AUC values for LAMB-LUAD CpGs.

| CpG | Gene | β_{LUAD, Mean} | β_{ANT, Mean} | Δβ_{Mean} | AUC_{LUAD/ANT} | AUC_{LUAD/Blood} | β_{Blood, Mean} |
|---|---|---|---|---|---|---|---|
| cg11835068 | AJAP1 | 0.2685057 | 0.08936861 | 0.17913713 | 0.9012101 | 0.9669900 | 0.076347668 |
| cg13495205 | AJAP1 | 0.2744210 | 0.08337360 | 0.19104744 | 0.8466574 | 0.9595600 | 0.075531512 |
| cg17741689 | AJAP1 | 0.1857749 | 0.07083236 | 0.11494253 | 0.8702638 | 0.8740667 | 0.074595268 |
| cg02792792 | CDO1 | 0.3086165 | 0.12532621 | 0.18329024 | 0.8716524 | 0.9941726 | 0.092619607 |
| cg08516516 | CDO1 | 0.2042318 | 0.02796895 | 0.17626284 | 0.9363222 | 0.9854071 | 0.021552865 |
| cg11036833 | CDO1 | 0.2638781 | 0.05130665 | 0.21257149 | 0.8910930 | 0.9942333 | 0.025920091 |
| cg14470895 | CDO1 | 0.2377576 | 0.06144272 | 0.17631490 | 0.9012101 | 0.9761925 | 0.047368004 |
| cg16707405 | CDO1 | 0.2171100 | 0.07208721 | 0.14502281 | 0.8803809 | 0.9869733 | 0.060589798 |
| cg23180938 | CDO1 | 0.2574357 | 0.04155912 | 0.21587660 | 0.9156913 | 0.9924001 | 0.020763679 |
| cg05471296 | HOXA7 | 0.2525723 | 0.10793649 | 0.14463582 | 0.8026185 | 0.9923272 | 0.061575115 |
| cg05743885 | HOXA7 | 0.2314730 | 0.09621283 | 0.13526014 | 0.8407062 | 0.9803567 | 0.068059061 |
| cg09017619 | HOXA7 | 0.1958601 | 0.06567961 | 0.13018050 | 0.8248363 | 0.9556387 | 0.048776390 |
| cg15797110 | HOXA7 | 0.2440474 | 0.10904027 | 0.13500713 | 0.8151160 | 0.9878231 | 0.074788188 |
| cg19153763 | HOXA7 | 0.2253907 | 0.11466550 | 0.11072518 | 0.8218607 | 0.9896077 | 0.079903406 |
| cg20293594 | HOXA7 | 0.2094773 | 0.08089118 | 0.12858609 | 0.8569728 | 0.9933106 | 0.049937194 |
| cg02643054 | HOXA9 | 0.3325415 | 0.19676219 | 0.13577927 | 0.8018250 | 0.9998543 | 0.083826778 |
| cg05065989 | HOXA9 | 0.1675814 | 0.04466354 | 0.12291786 | 0.9220393 | 0.9973048 | 0.014078937 |
| cg12600174 | HOXA9 | 0.3379927 | 0.09506522 | 0.24292749 | 0.9196588 | 0.9970741 | 0.021116643 |
| cg18447772 | HOXA9 | 0.3002316 | 0.08574388 | 0.21448769 | 0.9075580 | 0.9975598 | 0.017863377 |
| cg20741169 | HOXA9 | 0.2727795 | 0.13381630 | 0.13896316 | 0.8849435 | 0.9969770 | 0.093753567 |
| cg22055728 | HOXA9 | 0.2493123 | 0.13273871 | 0.11657356 | 0.8482444 | 0.9995508 | 0.037649644 |
| cg26365299 | HOXA9 | 0.1606362 | 0.04541882 | 0.11521740 | 0.8964491 | 0.9977661 | 0.009668684 |
| cg02191312 | PTGDR | 0.1214947 | 0.02855120 | 0.09294345 | 0.8881174 | 0.9607012 | 0.013546918 |
| cg17929687 | PTGDR | 0.2405110 | 0.07379570 | 0.16671527 | 0.8536005 | 0.9396982 | 0.053265954 |
| cg04672706 | SOX17 | 0.2056762 | 0.06297732 | 0.14269892 | 0.8539972 | 0.9269628 | 0.052638771 |
| cg24891539 | SOX17 | 0.2116766 | 0.09388336 | 0.11779329 | 0.8383257 | 0.8611128 | 0.097191438 |
| cg24928391 | SOX17 | 0.2605917 | 0.07639479 | 0.18419691 | 0.8353501 | 0.9226408 | 0.083656970 |
| cg02539083 | TAC1 | 0.1319065 | 0.06344382 | 0.06846271 | 0.8369371 | 0.9045393 | 0.060660579 |
| cg05157140 | UNCX | 0.2500439 | 0.07209194 | 0.17795195 | 0.8514184 | 0.9252753 | 0.062346567 |
| cg08507422 | UNCX | 0.1526641 | 0.04938463 | 0.10327943 | 0.8049990 | 0.9221673 | 0.036618037 |
| cg20870512 | UNCX | 0.2249826 | 0.08148049 | 0.14350216 | 0.8087681 | 0.8973036 | 0.071144868 |
| cg21127068 | ZIC4 | 0.2688158 | 0.08828222 | 0.18053361 | 0.9174767 | 0.9508189 | 0.071413684 |
| cg24686610 | ZIC4 | 0.3219377 | 0.11959429 | 0.20234343 | 0.9343384 | 0.9823198 | 0.092793728 |
| cg12388007 | ZIC4 | 0.1952284 | 0.11520781 | 0.08002055 | 0.8371355 | 0.9377314 | 0.093606493 |
| cg03611452 | ZNF781 | 0.2377772 | 0.08787763 | 0.14989960 | 0.8149177 | 0.9136447 | 0.045145657 |
| cg14587524 | ZNF781 | 0.2691666 | 0.06708518 | 0.20208138 | 0.8672882 | 0.8991004 | 0.049338765 |
| cg25324105 | ZNF781 | 0.2631115 | 0.08351355 | 0.17959795 | 0.8317794 | 0.9028761 | 0.051108829 |

CpG: Illumina HumanMethylation450K Manifest 450K identifier for CpG site; β_{LUAD, Mean}: geometric mean methylation frequency of paired lung adenocarcinomas (LUAD); β_{ANT, Mean}: geometric mean methylation frequency of paired adjacent noncancerous tissues (ANT); Δβ_{Mean}: difference between β_{LUAD, Mean} and β_{ANT, Mean}; AUC_{LUAD/ANT}: area under curve (AUC) for ROC curve between paired LUADs and ANTs; AUC_{LUAD/Blood}: area under curve (AUC) for ROC curve between separate paired LUADs and blood; β_{Blood, Mean}: geometric mean methylation frequency of remaining unpaired blood samples.

TABLE 14
Methylation frequency and AUC values for LAMB-LUSC CpGs.

| CpG | Gene | β_{LUSC, Mean} | β_{ANT, Mean} | Δβ_{Mean} | AUC_{LUSC/ANT} | AUC_{LUSC/Blood} | β_{Blood, Mean} |
|---|---|---|---|---|---|---|---|
| cg13495205 | AJAP1 | 0.30079819 | 0.04548144 | 0.25531674 | 0.9402872 | 0.8639997 | 0.07543220 |
| cg02792792 | CDO1 | 0.30941505 | 0.09475682 | 0.21465823 | 0.8954397 | 0.8740586 | 0.09262293 |
| cg08516516 | CDO1 | 0.19824624 | 0.01746244 | 0.18078380 | 0.9438146 | 0.8562730 | 0.02191331 |
| cg11036833 | CDO1 | 0.25523729 | 0.02779236 | 0.22744493 | 0.9178634 | 0.8984493 | 0.02554095 |
| cg05471296 | HOXA7 | 0.27398530 | 0.05811544 | 0.21586986 | 0.8440413 | 0.8554541 | 0.06110691 |
| cg05743885 | HOXA7 | 0.25246227 | 0.06834927 | 0.18411300 | 0.8591585 | 0.8597803 | 0.06816125 |
| cg09017619 | HOXA7 | 0.22899966 | 0.03327202 | 0.19572763 | 0.8715042 | 0.8131354 | 0.04813927 |
| cg10474350 | HOXA7 | 0.18404561 | 0.02735320 | 0.15669241 | 0.8735198 | 0.8122452 | 0.04048240 |
| cg15797110 | HOXA7 | 0.26023741 | 0.06309481 | 0.19714260 | 0.8596624 | 0.8203279 | 0.07465489 |
| cg19153763 | HOXA7 | 0.23911610 | 0.08627323 | 0.15284286 | 0.8523558 | 0.9105913 | 0.07990609 |
| cg20293594 | HOXA7 | 0.24720791 | 0.04577821 | 0.20142970 | 0.8828420 | 0.8461785 | 0.04978636 |
| cg02643054 | HOXA9 | 0.51007758 | 0.12935638 | 0.38072120 | 0.9821114 | 0.9750930 | 0.08360369 |
| cg05065989 | HOXA9 | 0.24022233 | 0.03042921 | 0.20979312 | 0.9974805 | 0.9897808 | 0.01417480 |
| cg12600174 | HOXA9 | 0.52969986 | 0.07498740 | 0.45471247 | 0.9972285 | 0.9950507 | 0.02103371 |
| cg16104915 | HOXA9 | 0.37437582 | 0.03732388 | 0.33705194 | 0.9997480 | 0.9931991 | 0.01243566 |
| cg18447772 | HOXA9 | 0.48869912 | 0.06256867 | 0.42613045 | 0.9984883 | 0.9956382 | 0.01827564 |
| cg20741169 | HOXA9 | 0.40260165 | 0.10922492 | 0.29337673 | 0.9964727 | 0.9788495 | 0.09346092 |
| cg22055728 | HOXA9 | 0.34810754 | 0.10149310 | 0.24661444 | 0.9934492 | 0.9822144 | 0.03759015 |
| cg02191312 | PTGDR | 0.05527317 | 0.01900974 | 0.03626343 | 0.8127992 | 0.8755007 | 0.01355779 |
| cg17929687 | PTGDR | 0.15476369 | 0.04077480 | 0.11398890 | 0.8505921 | 0.8009578 | 0.05327346 |
| cg24928391 | SOX17 | 0.29942179 | 0.04357265 | 0.25584914 | 0.9458302 | 0.8217166 | 0.08426630 |
| cg24896649 | ZFP42 | 0.28337686 | 0.07947542 | 0.20390145 | 0.9183673 | 0.8683437 | 0.07450728 |
| cg05548555 | ZIC4 | 0.26436477 | 0.09350979 | 0.17085498 | 0.8868733 | 0.9096299 | 0.08978951 |
| cg06369327 | ZIC4 | 0.25185221 | 0.10559287 | 0.14625934 | 0.8488284 | 0.9135466 | 0.07638609 |
| cg21127068 | ZIC4 | 0.29376894 | 0.05202472 | 0.24174423 | 0.9538927 | 0.9314569 | 0.07127759 |
| cg03355998 | ZIC4 | 0.16361256 | 0.03915513 | 0.12445743 | 0.8589065 | 0.9018854 | 0.02277972 |
| cg12388007 | ZIC4 | 0.25998717 | 0.08355085 | 0.17643633 | 0.9483497 | 0.8939985 | 0.09361155 |
| cg03611452 | ZNF781 | 0.26273042 | 0.04472334 | 0.21800707 | 0.8888889 | 0.8826043 | 0.04386284 |
| cg14587524 | ZNF781 | 0.30803779 | 0.03543552 | 0.27260227 | 0.8911565 | 0.8707294 | 0.04869757 |
| cg25324105 | ZNF781 | 0.29479674 | 0.04296358 | 0.25183315 | 0.8788108 | 0.8667770 | 0.05006793 |

CpG: Illumina HumanMethylation450K Manifest 450K identifier for CpG site; β_{LUSC, Mean}: geometric mean methylation frequency of paired lung squamous cell carcinoma (LUSC); β_{ANT, Mean}: geometric mean methylation frequency of paired adjacent noncancerous tissues (ANT); Δβ_{Mean}: difference between β_{LUSC, Mean} and β_{ANT, Mean}; AUC_{LUSC/ANT}: area under curve (AUC) for ROC curve between paired LUSCs and ANTs ; AUC_{LUSC/Blood}: area under curve (AUC) for ROC curve between separate paired LUSCs and β_{Blood, Mean}: geometric mean methylation frequency of remaining unpaired blood samples

1. A method of diagnosing and treating hepatocellular carcinoma (HCC) in a patient, the method comprising:
a) measuring levels of methylation at one or more CpG sites in promoter regions of one or more biomarker genes in cell-free DNA (cfDNA) from blood sample obtained from the patient, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 compared to reference value ranges for methylation at the one or more CpG sites in the cfDNA indicate that the patient has the HCC; and
b) treating the patient for the HCC, if the patient has a positive diagnosis for the HCC based on the levels of methylation of the one or more CpG sites.

2. The method of claim 1, wherein the one or more CpG sites are selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 and CpG sites located within 200 nucleotides thereof.

3. The method of claim 2, wherein said measuring levels of methylation comprises measuring levels of methylation at the cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 CpG sites.

4. The method of claim 1, wherein said treating the patient for HCC comprises surgical resection of an HCC tumor, liver transplantation, radiofrequency ablation, cryoablation, radiation therapy, chemotherapy, immunotherapy, or biologic therapy.

5. The method of claim 1, wherein the reference value ranges for methylation at the one or more CpG sites are obtained from cfDNA from one or more blood samples from one or more control subjects not having HCC.

6. An in vitro method of diagnosing hepatocellular carcinoma (HCC) in a patient, the method comprising:
b) measuring levels of methylation at one or more CpG sites in promoter regions of one or more biomarker genes in cell-free DNA (cfDNA) from blood sample obtained from the patient, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 compared to reference value ranges for methylation at the one or more CpG sites in the cfDNA indicate that the patient has the HCC.

7. A method of monitoring hepatocellular carcinoma (HCC) in a patient gr monitoring for a recurrence of HCC in a patient and treating the patient for the recurrence, the method comprising:
a) measuring levels of methylation at one or more CpG sites in promoter regions of one or more biomarker genes in cell-free DNA (cfDNA) from a first blood sample obtained from the patient at a first time point, optionally obtained from the patient after treatment for a previous occurrence of HCC at a first time point when the patient is characterized as cancer free from imaging or other diagnostic modalities, wherein the one or more biomarker genes are selected from AK055957, APC, GSTP1, HOXA1, PFKP, PRDM2, RUNX3, SEPTIN9, SPINT2, and WIF1,
b) measuring levels of methylation at one or more CpG sites in promoter regions of one or more biomarker genes in cfDNA from a second blood sample obtained from the patient later at a second time point, optionally obtained from the patient at a second time point during a period of monitoring for the recurrence, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA from the second blood sample compared to the cfDNA from the first blood sample indicate that the HCC is progressing or has recurred, and wherein detection of decreased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 in the cfDNA from the second blood sample compared to the cfDNA from the first blood sample indicate that the HCC is not progressing or has not recurred,
c) optionally treating the patient for the recurrence of the HCC, if the patient has a positive diagnosis for the recurrence of the HCC based on the levels of methylation of the one or more CpG sites; and
d) optionally repeating step b) subsequently during the period of monitoring for the recurrence.

8. The method of claim 7, wherein the HCC is a primary tumor, a metastasis, or a recurrence.

9. The method of claim 7, wherein the first time point is before a treatment of the patient for HCC is started and the second time point is during or after the treatment.

10. The method of claim 9, wherein the treatment is surgical resection, liver transplantation, radiofrequency ablation, cryoablation, radiation therapy, chemotherapy, immunotherapy, or biologic therapy.

11. The method of claim 7, further comprising repeating steps a) and b).

12. The method of claim 7, further comprising increasing dosage or frequency of a treatment for HCC, changing to a different treatment, or starting palliative care for the patient if the HCC is progressing.

13. The method of claim 7, wherein the one or more CpG sites are selected from cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 and CpG sites located within 200 nucleotides thereof.

14. The method of claim 13, wherein said measuring levels of methylation comprises measuring levels of methylation at the cg08572734, cg15607538, cg08571859, cg14479889, cg03667968, cg00577935, cg02659086, cg26744375, cg09420439, cg04673590, cg08465862, cg14250130, cg00922376, cg05346841, cg13629563, cg26421310, cg17300544, cg06848185, cg22522066, cg26397188, and cg24166864 CpG sites.

15-17. (canceled)

18. The method of claim 7, wherein said treating the patient for the recurrence of HCC comprises surgical resection of an HCC tumor, liver transplantation, radiofrequency ablation, cryoablation, radiation therapy, chemotherapy, immunotherapy, or biologic therapy.

19. The method of claim 1, further comprising measuring levels of alphafetoprotein (AFP).

20. (canceled)

21. A method of diagnosing and, optionally treating hepatocellular carcinoma (HCC), colorectal adenocarcinoma (CRC), prostate adenocarcinoma (PRAD), lung adenocarcinoma (LUAD), or lung squamous cell carcinoma (LUSC) in a patient, the method comprising:
(a)(i) measuring levels of methylation at one or more CpG sites in promoter regions of one or more biomarker genes in cell-free DNA (cfDNA) from a blood sample obtained from the patient, wherein the one or more biomarker genes are selected from AK055957, APC, GSTP1, HOXA1, PFKP, PRDM2, RUNX3, SEPTIN9, SPINT2, and WIF1, wherein increased levels of methylation at the one or more CpG sites in the promoter regions of the one or more biomarker genes selected from AK055957, APC, GSTP1, HOXA1, PFKP, PRDM2, RUNX3, SEPTIN9, SPINT2, and WIF1 compared to reference value ranges for methylation at the one or more CpG sites in the cfDNA indicate that the patient has the HCC
(a)(ii) measuring levels of methylation at one or more CpG sites in promoter regions of one or more biomarker genes in cell-free DNA (cfDNA) from a blood sample obtained from the patient, wherein the one or more biomarker genes are selected from ALX4, CNRIP1, COL4A1, COL4A2, EFEMP1, EYA4, FBN1, HIC1, IKZF1, MIR34C, NDRG4, SDC2, SEPTIN9, SOX21, TFP12, TMEFF2, and UNC5C, wherein increased levels of methylation at the one or more CpG sites in the promoter regions of the one or more biomarker genes selected from ALX4, CNRIP1, COL4A1, COL4A2, EFEMP1, EYA4, FBN1, HIC1, IKZF1, MIR34C, NDRG4, SDC2, SEPTIN9, SOX21, TFPI2, TMEFF2, and UNC5C compared to reference value ranges for methylation at the one or more CpG sites in cfDNA indicate that the patient has the CRC;
(a)(iii) measuring levels of methylation at one or more CpG sites in promoter regions of one or more biomarker genes in cell-free DNA (cfDNA) from a blood sample obtained from the patient, wherein the one or more biomarker genes are selected from APC, CD44, PYCARD, RARB, and RBP1, wherein increased levels of methylation at the one or more CpG sites in the promoter regions of the one or more biomarker genes selected from APC, CD44, PYCARD, RARB, and RBP1 compared to reference value ranges for methylation at the one or more CpG sites in cfDNA indicate that the patient has the PRAD;
(a)(iv) measuring levels of methylation at one or more CpG sites in promoter regions of one or more biomarker genes in cell-free DNA (cfDNA) from a blood sample obtained from the patient, wherein the one or more biomarker genes are selected from AJAP1, CDO1, HOXA7, HOXA9, PTGDR, SOX17, TAC1, UNCX, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, TAC1, UNCX, ZIC4, and ZNF781 compared to reference value ranges for methylation at the one or more CpG sites in cfDNA indicate that the patient has the LUAD; and
(a)(v) measuring levels of methylation at one or more CpG sites in promoter regions of one or more biomarker genes in cell-free DNA (cfDNA) from a blood sample obtained from the patient, wherein the one or more biomarker genes are selected from AJAP1, CDO1, HOXA7, HOXA9, PTGDR, SOX17, ZFP42, ZIC4, and ZNF781, wherein increased levels of methylation at the one or more CpG sites in the promoter regions of the one or more biomarker genes selected from AJAP1, CDO1, HOXA7, HOXA9, PTGDR, SOX17, ZFP42, ZIC4, and ZNF781 compared to reference value ranges for methylation at the one or more CpG sites in cfDNA indicate that the patient has the LUSC;
and
b) optionally treating the patient for the HCC, CRC, PRAD, LUAD, or LUSC, if the patient has a positive diagnosis for the HCC, CRC, PRAD, LUAD, or LUSC based on the levels of methylation of the one or more CpG sites.

22-98. (canceled)
